SEQUENCE LISTING (57 sequences)

SEQ ID NO: 1 - RNA, 633 nt, Artificial Sequence
  IL15RA+LINKER+IL15
attacgtgcccgccaccgatgtctgtcgagcacgcagatatttgggttaaatcctacagtctgtactcccgtgagcggtacatctgtaattcggggttcaaacggaaggctggtacctcgtcgctgacagaatgtgtgctcaataaggcaaccaacgtggcccactggacaaccccatcgctcaagtgcatcagggaccctgctctcgttcatcagcgccccgcaccaccttccggcgggtcgggaggcggcggaagtggaggcggttctggaggtggtggctcgctgcaaaactgggtgaatgtgatttccgatctcaagaaaattgaggaccttatccagtctatgcacatcgatgcgacactgtacacggagtcggacgtccacccttcttgcaaagtgactgctatgaagtgcttcttgttggagttgcaagtgatcagccttgaatcgggtgacgcttctattcacgataccgtcgaaaacctgatcatcctcgctaataactcgctttcctctaatggtaatgttaccgagtccggctgtaaggaatgcgaggaactcgaagagaagaacattaaggagttcctccagagctttgttcatatcgttcaaatgttcatcaacacgtcc

SEQ ID NO: 2 - protein, 211 aa, Artificial Sequence
  IL15RA+LINKER+IL15
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - RNA, 231 nt, Homo sapiens
  IL15RA
atcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccc

SEQ ID NO: 4 - protein, 77 aa, Homo sapiens
  IL15RA
ITCPPPMSVEHADIWVKSYSLYSRERYICNSGFKRKAGTSSLTECVLNKATNVAHWTTPSLKCIRDPALVHQRPAPP

SEQ ID NO: 5 - RNA, 342 nt, Homo sapiens
  IL15
aactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 6 - protein, 114 aa, Homo sapiens
  IL15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - RNA, 60 nt, Artificial Sequence
  Linker
agtggaggcagcggaggcggaggctccggcggaggaagtggaggcggaggcagtctgcag

SEQ ID NO: 8 - protein, 20 aa, Artificial Sequence
  Linker
SGGSGGGGSGGGSGGGGSLQ

SEQ ID NO: 9 - RNA, 66 nt, Toxoplasma <Apicomplexa>
  SUB1
gggtcatctcatgccattgtcgcgtgtgctgccttgatcgtgttgttatccaccaacgcgagagga

SEQ ID NO: 10 - protein, 22 aa, Toxoplasma <Apicomplexa>
  SUB1
GSSHAIVACAALIVLLSTNARG

SEQ ID NO: 11 - RNA, 585 nt, Toxoplasma <Apicomplexa>
  SUB1 propeptide
ctcagggtgcggaaagacaaagacgtactgttggcaacgagttttctttcccaccatggagaatatcagaatcccactggcacatataaccttatcaaggagattcggagggtggaagcggaaatcgaagatgaggttgaaacactaaacagagacagaaggctgcatcgtgggcacaacaaatatgccgatgacgacatccgacaggggctaaagggcgagcaagatatgggcgcttcagaaaatattcccgttgcggagctcgaaccacaggatttagatcgggaggcgaagtatcctgtgcggatgttgattgtggacaagcgttctgatgatgatgatgaagagaccaaaacctcgtttgtggagacggcgctacattcggatcttgcgcaacgcgttgtaaaggagctgaacgggcatgtggacgtccttagagaaagtggcgtggttttagtggatcttccagcacaaaccaccgacaaacaactgcaggaattgatcgaaaccgccagggcacaaggcaccatagtagagccggatcacttggttcaaagtgtcaatacatcgtcgaaaggatcaaat

SEQ ID NO: 12 - protein, 195 aa, Toxoplasma <Apicomplexa>
  SUB1 propeptide
LRVRKDKDVLLATSFLSHHGEYQNPTGTYNLIKEIRRVEAEIEDEVETLNRDRRLHRGHNKYADDDIRQGLKGEQDMGASENIPVAELEPQDLDREAKYPVRMLIVDKRSDDDDEETKTSFVETALHSDLAQRVVKELNGHVDVLRESGVVLVDLPAQTTDKQLQELIETARAQGTIVEPDHLVQSVNTSSKGSN

SEQ ID NO: 13 - RNA, 84 nt, Toxoplasma <Apicomplexa>
  MIC5 signal sequence
ctgcgacctactgttcgtagctcgttactgtccctggggctgacagtgatcctctatctggctcttaccggctctgctgatgcg

SEQ ID NO: 14 - protein, 28 aa, Toxoplasma <Apicomplexa>
  MIC5 signal sequence
LRPTVRSSLLSLGLTVILYLALTGSADA

SEQ ID NO: 15 - RNA, 54 nt, Toxoplasma <Apicomplexa>
  MIC5 propeptide
ttagcctcgcatttacgctccagacacatggaagccggaagacgaaccatggat

SEQ ID NO: 16 - protein, 18 aa, Toxoplasma <Apicomplexa>
  MIC5 propeptide
LASHLRSRHMEAGRRTMD

SEQ ID NO: 17 - RNA, 78 nt, Toxoplasma <Apicomplexa>
  GPI anchor
gctgcgggaacagccagtcacgtttccatttttgccatggtgatcggacttattggctctatcgcagcttgtgtcgcg

SEQ ID NO: 18 - protein, 26 aa, Toxoplasma <Apicomplexa>
  GPI anchor
AAGTASHVSIFAMVIGLIGSIAACVA

SEQ ID NO: 19 - RNA, 738 nt, Artificial Sequence
  ScFv
caggtgcagctgcaggagagtggcggaggactggtgcagccaggcggaagtctgcgcctgagttgcgccgccagcggcttcaccttcaacgatttctacatgaactggattcgccagcccccaggccaggccccagagtggctgggagtgattcgcaacaagggcaacggctacaccaccgaagtgaacaccagcgtgaagggacgcttcaccatcagccgcgataacacccagaacatcctgtacttgcagatgaacagcctgcgcgccgaggataccgccatctactattgcgcccgcggcggcccctactactacagcggagatgatgccccctactggggccagggcgtgatggtcaccgtgtccagtggcggaggcggatccggcggaggcggaagcggaggcggaggcagtgacatccagatgacccagagccccagttccctgagcgccagtgtgggagatcgcgtgaccattacctgccgcgcctcgcagaacatcaagggctggctggcctggtatcagcagaagtccggaaacgccccccagctgctgatctacaaggccagcagtttgcagagcggcgtgcccagccgctttagcggaagtggaagcggcaccgattacatcttcaccattagcaacttgcagcccgaggacattgccacctactactgccagcactaccagagcttcccgtggaccttcggcggaggtaccaagctggagctgaag

SEQ ID NO: 20 - protein, 246 aa, Artificial Sequence
  ScFv
QVQLQESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSGGGGSGGGGSGGGGSDIQMTQSPSSLSASVGDRVTITCRASQNIKGWLAWYQQKSGNAPQLLIYKASSLQSGVPSRFSGSGSGTDYIFTISNLQPEDIATYYCQHYQSFPWTFGGGTKLELK

SEQ ID NO: 21 - RNA, 99 nt, Toxoplasma <Apicomplexa>
  SAG1 Nterm
atgtttccgaaggcagtgagacgcgccgtcacggcaggggtgtttgccgcgcccacactgatgtcgttcttgcgatgtggcgttatggcatcggatccc

SEQ ID NO: 22 - protein, 33 aa, Toxoplasma <Apicomplexa>
  SAG1 Nterm
MFPKAVRRAVTAGVFAAPTLMSFLRCGVMASDP

SEQ ID NO: 23 - RNA, 756 nt, Toxoplasma <Apicomplexa>
  SAG1 without N terminal signal sequence
cctcttgttgccaatcaagttgtcacctgcccagataaaaaatcgacagccgcggtcattctcacaccgacggagaaccacttcactctcaagtgccctaaaacagcgctcacagagcctcccactcttgcgtactcacccaacaggcaaatctgcccagcgggtactacaagtagctgtacatcaaaggctgtaacattgagctccttgattcctgaagcagaagatagctggtggacgggggattctgctagtctcgacacggcaggcatcaaactcacagttccaatcgagaagttccccgtgacaacgcagacgtttgtggtcggttgcatcaagggagacgacgcacagagttgtatggtcacagtgacagtacaagccagagcctcatcggtcgtcaataatgtcgcaaggtgctcctacggtgcagacagcactcttggtcctgtcaagttgtctgcggaaggacccactacaatgaccctcgtgtgcgggaaagatggagtcaaagttcctcaagacaacaatcagtactgttccgggacgacgctgactggttgcaacgagaaatcgttcaaagatattttgccaaaattaactgagaacccgtggcagggtaacgcttcgagtgataagggtgccacgctaacgatcaagaaggaagcatttccagccgagtcaaaaagcgtcattattggatgcacagggggatcgcctgagaagcatcactgtaccgtgaaactggagtttgcc

SEQ ID NO: 24 - protein, 252 aa, Toxoplasma <Apicomplexa>
  SAG1 without N terminal signal sequence
PLVANQVVTCPDKKSTAAVILTPTENHFTLKCPKTALTEPPTLAYSPNRQICPAGTTSSCTSKAVTLSSLIPEAEDSWWTGDSASLDTAGIKLTVPIEKFPVTTQTFVVGCIKGDDAQSCMVTVTVQARASSVVNNVARCSYGADSTLGPVKLSAEGPTTMTLVCGKDGVKVPQDNNQYCSGTTLTGCNEKSFKDILPKLTENPWQGNASSDKGATLTIKKEAFPAESKSVIIGCTGGSPEKHHCTVKLEFA

SEQ ID NO: 25 - RNA, 27 nt, Artificial Sequence
  HA Tag
tacccatacgatgttccagattacgct

SEQ ID NO: 26 - protein, 9 aa, Artificial Sequence
  HA Tag
YPYDVPDYA

SEQ ID NO: 27 - RNA, 15 nt, Artificial Sequence
  Linker
ggcggaggcgctagc

SEQ ID NO: 28 - protein, 5 aa, Artificial Sequence
  Linker
GGGAS

SEQ ID NO: 29 - protein, 295 aa, Artificial Sequence
  DC2
MFPKAVRRAVTAGVFAAPTLMSFLRCGVMASDPTSYPYDVPDYAQVQLQESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSGGGGSGGGGSGGGGSDIQMTQSPSSLSASVGDRVTITCRASQNIKGWLAWYQQKSGNAPQLLIYKASSLQSGVPSRFSGSGSGTDYIFTISNLQPEDIATYYCQHYQSFPWTFGGGTKLELKGGGAS

SEQ ID NO: 30 - protein, 549 aa, Artificial Sequence
  DC2
MFPKAVRRAVTAGVFAAPTLMSFLRCGVMASDPTSYPYDVPDYAQVQLQESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSGGGGSGGGGSGGGGSDIQMTQSPSSLSASVGDRVTITCRASQNIKGWLAWYQQKSGNAPQLLIYKASSLQSGVPSRFSGSGSGTDYIFTISNLQPEDIATYYCQHYQSFPWTFGGGTKLELKGGGASSRPLVANQVVTCPDKKSTAAVILTPTENHFTLKCPKTALTEPPTLAYSPNRQICPAGTTSSCTSKAVTLSSLIPEAEDSWWTGDSASLDTAGIKLTVPIEKFPVTTQTFVVGCIKGDDAQSCMVTVTVQARASSVVNNVARCSYGADSTLGPVKLSAEGPTTMTLVCGKDGVKVPQDNNQYCSGTTLTGCNEKSFKDILPKLTENPWQGNASSDKGATLTIKKEAFPAESKSVIIGCTGGSPEKHHCTVKLEFA

SEQ ID NO: 31 - protein, 326 aa, Artificial Sequence
  DC2
MFPKAVRRAVTAGVFAAPTLMSFLRCGVMASDPTSYPYDVPDYAQVQLQESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSGGGGSGGGGSGGGGSDIQMTQSPSSLSASVGDRVTITCRASQNIKGWLAWYQQKSGNAPQLLIYKASSLQSGVPSRFSGSGSGTDYIFTISNLQPEDIATYYCQHYQSFPWTFGGGTKLELKGGGASSREDLAAGTASHVSIFAMVIGLIGSIAACVA

SEQ ID NO: 32 - protein, 580 aa, Artificial Sequence
  DC2-SAG1
MFPKAVRRAVTAGVFAAPTLMSFLRCGVMASDPTSYPYDVPDYAQVQLQESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSGGGGSGGGGSGGGGSDIQMTQSPSSLSASVGDRVTITCRASQNIKGWLAWYQQKSGNAPQLLIYKASSLQSGVPSRFSGSGSGTDYIFTISNLQPEDIATYYCQHYQSFPWTFGGGTKLELKGGGASSRPLVANQVVTCPDKKSTAAVILTPTENHFTLKCPKTALTEPPTLAYSPNRQICPAGTTSSCTSKAVTLSSLIPEAEDSWWTGDSASLDTAGIKLTVPIEKFPVTTQTFVVGCIKGDDAQSCMVTVTVQARASSVVNNVARCSYGADSTLGPVKLSAEGPTTMTLVCGKDGVKVPQDNNQYCSGTTLTGCNEKSFKDILPKLTENPWQGNASSDKGATLTIKKEAFPAESKSVIIGCTGGSPEKHHCTVKLEFASREDLAAGTASHVSIFAMVIGLIGSIAACVA

SEQ ID NO: 33 - protein, 765 aa, Artificial Sequence
  ScFv and HA
TACCCATACGATGTTCCAGATTACGCTCAGGTGCAGCTGCAGGAGAGTGGCGGAGGACTGGTGCAGCCAGGCGGAAGTCTGCGCCTGAGTTGCGCCGCCAGCGGCTTCACCTTCAACGATTTCTACATGAACTGGATTCGCCAGCCCCCAGGCCAGGCCCCAGAGTGGCTGGGAGTGATTCGCAACAAGGGCAACGGCTACACCACCGAAGTGAACACCAGCGTGAAGGGACGCTTCACCATCAGCCGCGATAACACCCAGAACATCCTGTACTTGCAGATGAACAGCCTGCGCGCCGAGGATACCGCCATCTACTATTGCGCCCGCGGCGGCCCCTACTACTACAGCGGAGATGATGCCCCCTACTGGGGCCAGGGCGTGATGGTCACCGTGTCCAGTGGCGGAGGCGGATCCGGCGGAGGCGGAAGCGGAGGCGGAGGCAGTGACATCCAGATGACCCAGAGCCCCAGTTCCCTGAGCGCCAGTGTGGGAGATCGCGTGACCATTACCTGCCGCGCCTCGCAGAACATCAAGGGCTGGCTGGCCTGGTATCAGCAGAAGTCCGGAAACGCCCCCCAGCTGCTGATCTACAAGGCCAGCAGTTTGCAGAGCGGCGTGCCCAGCCGCTTTAGCGGAAGTGGAAGCGGCACCGATTACATCTTCACCATTAGCAACTTGCAGCCCGAGGACATTGCCACCTACTACTGCCAGCACTACCAGAGCTTCCCGTGGACCTTCGGCGGAGGTACCAAGCTGGAGCTGAAG

SEQ ID NO: 34 - DNA, 33 bp, Artificial Sequence
  Primer
ggttttgacgtcaccatgtttccgaaggcagtg

SEQ ID NO: 35 - DNA, 32 bp, Artificial Sequence
  Primer
ttgctcaccatcctaggtgcagccccggcaaa

SEQ ID NO: 36 - DNA, 32 bp, Artificial Sequence
  primer
tttgccggggctgcacctaggatggtgagcaa

SEQ ID NO: 37 - DNA, 33 bp, Artificial Sequence
  primer
cggtgattaattaatcgagcgggtcctggttcg

SEQ ID NO: 38 - DNA, 86 bp, Artificial Sequence
  primer
gcggccaagcttataacttcgtataatgtatgctatacgaagttatgatatgcatgtccgcgttcgtgaaatctctgatcaagcgg

SEQ ID NO: 39 - DNA, 85 bp, Artificial Sequence
  primer
cgacgcacgctgtcactcaacttgctgctagaactagtggatccataacttcgtatagcatacattatacgaagttatccctcgg

SEQ ID NO: 40 - DNA, 21 bp, Artificial Sequence
  primer
gtatcgataagcttataactc

SEQ ID NO: 41 - DNA, 33 bp, Artificial Sequence
  primer
cacaacggtgattaacctaggagccccgccctg

SEQ ID NO: 42 - DNA, 28 bp, Artificial Sequence
  primer
caaacacctagggatcaagccagagagc

SEQ ID NO: 43 - DNA, 25 bp, Artificial Sequence
  primer
gttccctaggggaaacacatctgcc

SEQ ID NO: 44 - DNA, 35 bp, Artificial Sequence
  primer
ggtgctcaccggtttaaacgtcgaaaatgtttccg

SEQ ID NO: 45 - DNA, 28 bp, Artificial Sequence
  primer
ctcaccattctagaggaaacacatctgc

SEQ ID NO: 46 - DNA, 29 bp, Artificial Sequence
  primer
ctcatctctagagaggatctggctgcggg

SEQ ID NO: 47 - DNA, 27 bp, Artificial Sequence
  primer
accatggaagcggccgcttacgcgaca

SEQ ID NO: 48 - DNA, 24 bp, Artificial Sequence
  primer
ggcaacactagtgggatccgatgc

SEQ ID NO: 49 - DNA, 59 bp, Artificial Sequence
  primer
ctcgggactagttacccatacgatgttccagattacgctcaggtgcagctgcaggagag

SEQ ID NO: 50 - DNA, 37 bp, Artificial Sequence
  primer
attggccactctagagctagcgcctccgcccttcagc

SEQ ID NO: 51 - DNA, 30 bp, Artificial Sequence
  primer
atggcatcgtctagacctcttgttgccaat

SEQ ID NO: 52 - DNA, 27 bp, Artificial Sequence
  primer
cctaggtgctctagaggcaaactccag

SEQ ID NO: 53 - DNA, 33 bp, Artificial Sequence; identical to SEQ ID NO: 34
  primer

SEQ ID NO: 54 - protein, 32 aa, Artificial Sequence
  primer
TTGCTCACCATCCTAGGTGCAGCCCCGGCAAA

SEQ ID NO: 55 - protein, 15 aa, Artificial Sequence
  linker
GGGGSGGGGSGGGGS

SEQ ID NO: 56 - DNA, 741 bp, Artificial Sequence
  scFv
gaggtgcaactcgtcgaaagcgggggcggtttggtgcaacctgggggaagcctgcggttgtcttgcgccgcaagcggctttacgttttccgattcgtggattcattgggtgagacaagccccaggtaaggggctcgaatgggtggcgtggatcagtccgtatggtggatcgacttattacgcggactctgtgaaaggaaggtttacaatctccgcggatacgtccaaaaataccgcatatttgcagatgaatagccttcgcgcagaggacacagcagtttattactgcgcccggagacattggccaggcggcttcgattactgggggcaaggtacgctggttacagttagcagcgggggaggaggatctgggggaggtgggtcgggagggggaggttccgacatccaaatgactcagtcgccatctagtctttctgcctctgtgggggatcgtgttaccatcacgtgccgtgccagccaggacgttagcactgctgtggcctggtaccagcaaaagccggggaaggcacccaaacttctgatctatagtgcgtcgttcctctatagtggcgttccgtcgcgcttctctggtagtggctccggcaccgactttaccctgacaatcagtagcctgcagcctgaggacttcgctacctattattgccaacaatacctgtaccacccggcaaccttcggtcaaggaaccaaagtggagattaaaggagggggggccagttccaga

SEQ ID NO: 57 - protein, 247 aa, Artificial Sequence
  scFv
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGASSR

(19) United States
(12) Patent Application Publication
(10) Pub. No.: US 2022/0233607 A1
(43) Pub. Date: Jul. 28, 2022

(54) TOXOPLASMA PLATFORM FOR TREATING CANCER

(71) Applicants: UNIVERSITÉ DE TOURS, Tours (FR); KYMERIS THERAPEUTICS INC., Toronto (CA); INSTITUT NATIONAL DE RECHERCHE POUR L'AGRICULTURE, L'ALIMENTATION ET L'ENVIRONNEMENT, Paris (FR)

(72) Inventors: Isabelle DIMIER-POISSON, Tours (FR); Marie-Noëlle MEVELEC, Tours (FR); Anne DI TOMMASO, La Riche (FR); Nicolas AUBREY, Truyes (FR); Louis LANTIER, Notre-Dame-d'Oé (FR); Gordon LEE, Miami, FL (US)

(73) Assignees: UNIVERSITÉ DE TOURS, Tours (FR); KYMERIS THERAPEUTICS INC., Toronto (CA); INSTITUT NATIONAL DE RECHERCHE POUR L'AGRICULTURE, L' ALIMENTATION ET L'ENVIRONNEMENT, Paris (FR)

(21) Appl. No.: 17/614,771

(22) PCT Filed: May 29, 2020
(86) PCT No.: PCT/EP2020/065086

§ 371 Date: Nov. 29, 2021

(30) Foreign Application Priority Data
May 29, 2019 (EP) 19305686.8

(51) Int. Cl. A61K 35/68; C07K 14/54; C07K 14/715; C07K 14/77; C07K 16/28

(52) U.S. Cl. CPC A61K 35/68; C07K 14/5443; A61K 2039/505; C07K 14/77; C07K 16/2851; C07K 14/7155

(57) ABSTRACT

A strain of an Apicomplexa of the family Sarcocystidae, wherein the strain is replicative and expresses one or more heterologous protein(s) selected from the group including therapeutic proteins, antigens, recombinant surface receptor or combinations thereof, and wherein the strain is selected from the group including Toxoplasma gondii and Neospora caninium. Also, the use of the strain for preventing or treating cancers or infectious diseases in a subject in need thereof.

### FIELD OF INVENTION

The present invention relates to a strain of an Apicomplexa of the family Sarcocystidae wherein the strain is replicative and expresses one or more heterologous protein(s) such as therapeutic proteins, antigens, recombinant surface receptor and others. The present invention also relates to the use of said strain for preventing or treating cancers or infectious diseases in a subject in need thereof.

### BACKGROUND OF INVENTION

Cancers emerge after the immune system fails to control and contain tumors. Indeed, immune system suppresses cancer development by halting the replication of tumor cells and killing tumors cells. However, this constant immune attack of the tumor also triggers adaptation by tumor cells that create a tolerogenic-tumor environment that disarms the tumor killing potential of the immune system. Therefore, one way for treating cancers is to stimulate a long-term effective immune response against tumor.

For centuries acute inflammation associated with infection has been observationally linked with the spontaneous elimination of tumors suggesting that the stimulation of the immune system by microorganisms could be exploited to combat cancer. Indeed, as strong inducers of Th1-oriented immune responses, parasites may act as powerful adjuvants and may also serve as vaccine vectors for potentiating a specific T cell response against tumor antigens.

The beneficial adjuvant effect of intracellular parasite infection has been reported in several experimental models such as, melanoma-bearing mice, ovarian cancer-bearing mice and pancreatic tumor-bearing mice. For example, Baird et al. shown that injection of attenuated Toxoplasma gondii mutants at the tumor site may stimulate both the innate and adaptive responses, thereby leading to tumor regression.

Toxoplasma gondii is an obligatory intracellular protozoan parasite capable of infecting most warm-blooded vertebrates and many nucleated cell types responsible for human and animal toxoplasmosis. It belongs to the family Sarcocystidae, which also groups together other major pathogens of animals, such as Neospora caninum. Despite being taxonomically close to Toxoplasma gondii, Neospora caninum presents several differences with Toxoplasma gondii.

Another potential advantage of intracellular parasites is that they may be genetically engineered to express, for example, a tumor antigen allowing thereby creating a tumor vaccine vector able to license T cells to specifically kill tumor cells.

Actually, existing strategies use non-replicating versions of parasites which have the advantage of being nonpathogenic and safely tolerated. However, said non-replicative strains are less immunogenic and quickly cleared by the immune system.

In this present invention, the inventors showed that the administration of a replicative and genetically modified Apicomplexa of the family Sarcocystidae to a subject can induces a strong immune response, in particular against tumors. Indeed, the strain of the present invention is replicative and thus able to induce a strong immune response. Moreover, said Apicomplexa strain is genetically modified to express one or more heterologous proteins such as therapeutic proteins, antigens, recombinant surface receptor. The present invention thus aims at providing a new treatment for cancer or infectious diseases based on the use of replicative Toxoplasma gondii and Neospora caninum strains expressing heterologous proteins.

### SUMMARY

The present invention relates to a strain of an Apicomplexa of the family Sarcocystidae, wherein said strain is replicative and expresses at least one heterologous gene or protein.

In one embodiment, the strain is Toxoplasma gondii.

In one embodiment, the strain is Neospora caninum.

In one embodiment, the strain expresses and/or secretes one or more heterologous protein(s) selected from the group comprising therapeutic molecules, antigens, recombinant surface receptors, or combinations thereof.

In one embodiment, the therapeutic molecule is a cytokine, preferably a human cytokine, more preferably a human IL15Rα sushi.

In one embodiment, the antigen is a cancer antigen or a neoantigen.

In one embodiment, the recombinant surface receptor comprises at least one extracellular-binding domain.

In one embodiment, the at least one extracellular-binding domain is an antigen-binding fragment or an antibody selected from the group comprising whole antibody, humanized antibody, single chain antibody, dimeric single chain antibody, Fv, scFv, Fab, F(ab)′2, defucosylated antibody, bi-specific antibody, diabody, triabody, tetrabody surface-exposed binding domain.

In one embodiment, the antigen-binding fragment or antibody is a scFV, preferably a scFV directed to DEC205.

In one embodiment, the strain is at a tachyzoite stage.

The present invention further relates to a composition comprising the strain of the invention.

In one embodiment, the composition is a pharmaceutical composition and further comprises at least one pharmaceutically acceptable excipient.

In one embodiment, the composition as described above is in combination with a therapeutic protein or molecule.

The present invention further relates to a vaccine composition comprising the strain of the invention.

In one embodiment, the vaccine composition comprises an adjuvant.

In one embodiment, the strain as described herein, the composition as described herein or the vaccine composition as described herein for use in preventing and/or treating cancer or a chronic infectious disease.

In one embodiment, the chronic infectious disease is selected from chronic virus infection and chronic bacterial infection.

In one embodiment, the cancer is a solid tumor, preferably an ovarian cancer, pancreatic cancer, lung cancer, melanoma or glioblastoma.

In one embodiment, the chronic infectious disease is associated with or induces an immunosuppression, and is selected from the group consisting of tuberculosis and HIV.

In one embodiment, the strain, composition or vaccine is to be administered to the subject via subcutaneous, intradermal, intraperitoneal, intravaginal or intratumoral routes.

The present invention further relates to a method of producing at least one heterologous protein by a strain of the invention, said method comprising:
a) infecting a cell with the strain, wherein the strain secretes said at least one heterologous protein,
b) cultivating the infected cell of a) in a culture medium,
c) recovering the least one heterologous protein.

### Definitions

In the present invention, the following terms have the following meanings:
“About” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.
“Activated cells” refers to the state of an immune cell, in particular a T cell, which has been sufficiently stimulated to induce a detectable cellular response. Activation can also be associated with detectable effector function(s) such as cytokine production or suppressive activity.
“Antigen” refers to a substance that is recognized and selectively bound by an antibody or by a T cell antigen receptor, in order to trigger an immune response. It is contemplated that the term antigen encompasses native antigen as well as fragment (e.g., epitopes, immunogenic domains, etc.) and derivative thereof, provided that such fragment or derivative is capable of being the target of an immune response. Suitable antigens in the context of the invention are preferably polypeptides (e.g. peptides, polypeptides, post translationally modified polypeptides, etc.) including one or more B cell epitope(s) or one or more T cell epitope(s) or both B and T cell epitope(s) and capable of raising an immune response, preferably, a humoral or cell response that can be specific for that antigen. Typically, the one or more antigen(s) is selected in connection with the disease to treat.
“Autologous” refers to any material derived from the same individual to whom it is later to be re-introduced.
The term “homology” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. Thus, the term “homologous” or “identical”, when used in a relationship between the sequences of two or more polypeptides or of two or more nucleic acid molecules, refers to the degree of sequence relatedness between polypeptides or nucleic acid molecules, as determined by the number of matches between strings of two or more amino acid or nucleotide residues. “Identity” measures the percent of identical matches between the smaller of two or more sequences with gap alignments (if any) addressed by a particular mathematical model or computer program (i.e., “algorithms”) Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. \2, 387 (1984); Genetics Computer Group, University of Wisconsin, Madison, Wis.), BLASTP, BLASTN, and FASTA (Altschul et al., J. MoI. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.
“Heterologous” refers to nucleic acid molecule, and protein encoded bay said nucleic acid molecule originating outside the strain of the present invention. In other words, heterologous proteins are not naturally present in the strain of the invention. For example, heterologous nucleic acid molecule or heterologous protein can be any proteins that one would want to express within a mammalian host cell. The heterologous nucleic acid molecule of the invention encodes one or more heterologous protein.
“Immunodepletion” or “immunosuppression” refer to a deficient immune system, i.e., an immune system for which one or more cell lines are either absent or deficient.
As used herein, the term “immune cells” generally includes white blood cells (leukocytes) which are derived from hematopoietic stem cells (HSC) produced in the bone marrow. Examples of immune cells include, but are not limited to, lymphocytes (T cells, B cells, and natural killer (NK) cells) and myeloid-derived cells (neutrophil, eosinophil, basophil, monocyte, macrophage, dendritic cells).
As used herein, the term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages Immune cells involved in the immune response include lymphocytes, such as B cells and T cells (CD4⁺, CD8⁺, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes.
The term “isolated” means altered or removed from the natural state.
“Pharmaceutically acceptable excipient” refers to an excipient that does not produce an adverse, allergic or other untoward reaction when administered to an animal, preferably a human. It includes any and all dispersion media and solvents, coatings, isotonic and absorption delaying agents, additives, preservatives, stabilizers and the like. For human administration, preparations should meet sterility, pyrogenicity, general safety and purity standards as required by regulatory offices, such as, for example, FDA Office or EMA.
The term “specifically binds” refers to an antibody, or a ligand, which recognizes and binds with a binding partner present in a sample, but which antibody or ligand does not substantially recognize or bind other molecules in the sample.
“Subject” refers to a mammal, preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease. In one embodiment, the subject is an adult (for example a subject above the age of 18). In another embodiment, the subject is a child (for example a subject below the age of 18). In one embodiment, the subject is a male. In another embodiment, the subject is a female.
“Substantially purified” refers to a cell or strain (e.g., a strain Neospora caninum) that is essentially free of other cell types or organisms (e.g., of other protozoan organisms). In one embodiment, a substantially purified strain refers to a strain which is at least about 75% free, 80% free, or 85% free, and preferably about 90%, 95%, 96%, 97%, 98%, or 99% free, from other cell types or organisms.
“Therapeutically effective amount” refers to level or amount of agent that is aimed at, without causing significant negative or adverse side effects to the target, (1) delaying or preventing the onset of the targeted pathologic condition or disorder; (2) slowing down or stopping the progression, aggravation, or deterioration of one or more symptoms of the targeted pathologic condition or disorder; (3) bringing about ameliorations of the symptoms of the targeted pathologic condition or disorder; (4) reducing the severity or incidence of the targeted pathologic condition or disorder; (5) curing the targeted pathologic condition or disorder. An effective amount may be administered prior to the onset of the targeted pathologic condition or disorder, for a prophylactic or preventive action. Alternatively or additionally, the effective amount may be administered after initiation of the targeted pathologic condition or disorder, for a therapeutic action.
“Treatment” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject is successfully “treated” for the targeted pathologic condition or disorder if, after receiving a therapeutic amount of a strain of Neospora caninum or Toxoplasma gondii as described herein, the subject shows observable and/or measurable improvement in one or more of the following: reduction in the number of pathogenic cells; reduction in the percent of total cells that are pathogenic; relief to some extent of one or more of the symptoms associated with the targeted pathologic condition or disorder; reduced morbidity and mortality, and/or improvement in quality of life issues. The above parameters for assessing successful treatment and improvement in the disease are readily measurable by routine procedures familiar to a physician.
“Vaccine” refers to any preparation comprising substance or group of substances meant to induce an immune response in a subject, e.g., against a cancer cell, a tumor or against cells infected with an intracellular pathogen, such as, for example, Mycobacterium tuberculosis, HIV or plasmodium infected cells. As used herein, the term “vaccine” refers both to prophylactic vaccines and to therapeutic vaccines. Prophylactic vaccines are used to prevent a subject from the occurrence of a disease or condition (e.g., cancer or an infectious disease), or to limit the severity of the disease or condition, such that the subject administered with the vaccine only develops mild symptoms of the disease or condition. Therapeutic vaccines are intended to treat the targeted disease or condition, e.g., cancer or an infection disease, such as, for example, tuberculosis, HIV or malaria infections in a subject.

### DETAILED DESCRIPTION

The present invention relates to a strain of an Apicomplexa of the family Sarcocystidae, wherein said strain is replicative and expresses at least one heterologous gene or protein.

The strain according to the invention is a replicative and recombinant strain.

As used herein, the term “replicative” refers to a strain that infects, replicates and disseminates into mammalian host cells. Parasite replication can be determined by evaluating the number of parasites per vacuole over time using immunofluorescence staining for parasites and microscopic or flow cytometry-based analysis. Kinetic determination of parasite number per vacuole accurately reflects parasite replication over time as vacuoles-containing parasites do not fuse with one another.

As used herein, the term “expresses at least one heterologous gene or protein” refers to a strain of the invention that is engineered to express at least one heterologous nucleic acid molecule and comprises inserted in its genome at least one heterologous nucleic acid molecule of interest. According to the invention, the nucleic acid molecule of interest is a heterologous nucleic acid molecule to the host organism into which it is introduced. More specifically, it can be of human origin or not (e.g., of bacterial, yeast or viral origin). Advantageously, said nucleic acid of interest may encodes one or more heterologous proteins. A protein is understood to be any translational product of a polynucleotide regardless of size, and whether glycosylated or not, and includes peptides and proteins.

To obtain heterologous protein expression, the heterologous gene or the heterologous nucleic acid coding sequence of said heterologous proteins needs to be able to be expressed directly or indirectly from a recombinant molecule in the strain of the present invention. In this regard, it is desirable that the promoter employed is recognizable by the strain of the present invention. Moreover, it is desirable that the promoter promotes transcription of the protein coding sequence when the strain of the present invention is inside mammalian cells. Promoters (e.g., 5′ UTR, 3′ UTR, etc.) and other regulatory elements that can be used in the present invention include, without limitation, promoters and regulatory elements of Toxoplasma gondii and Neospora caninum. Known promoters which can be operably linked to the coding sequence of an heterologous protein of interest so that the heterologous protein is expressed in the strain of the invention include, but are not limited to, sequences from the αTUB5 or αTUB8 gene of Toxoplasma gondii, or all Toxoplasma or Apicomplexa promoters (stage-specific, ubiquitous, constitutive, tissue-specific, inducible . . . ) or synthetic promoters. Promoters for use in accordance with the present invention can also be stage-specific promoters, which selectively express the heterologous protein(s) of interest at different points in the obligate intracellular strain life cycle. Moreover, it is contemplated that an autologous promoter can be used to drive expression of the heterologous protein or antigen by, e.g., site-specific integration at the 3′ end of a known promoter in the strain of the invention. In one embodiment, a promoter of Toxoplasma gondii can be used to express the heterologous protein of interest in Neospora caninum. In another embodiment, a promoter of Neospora caninum can be used to express the heterologous protein of interest in Toxoplasma gondii.

In one embodiment, the strain according to the invention is attenuated.

As used herein, the term “attenuated” refers to a strain of the invention that can infect mammalian cells with less efficacy than a wild type strain. In other word, an attenuated strain takes longer to proliferate or replicate into mammalian cells than a wild type strain.

In a particular embodiment, the strain according to the invention is not attenuated by pyrimidine auxotrophy. In a particular embodiment, the attenuation of the strain of the invention is not related to an inactivation of the virulence genes.

As used herein, the term “inactivation of gene” denotes a genetic mutation resulting in a loss of function and/or a loss of expression of the protein encoded by the said gene. In one embodiment, said genetic mutation corresponds to the disruption of all or a portion of a gene of interest, preferably the total disruption of the gene. Preferably, the deletion starts at or before the start codon of the deleted gene, and ends at or after the stop codon of the deleted gene. Other examples of genetic mutations include, but are not limited to, substitution, deletion, or insertion.

The Strain of the invention is of an Apicomplexa of the family Sarcocystidae. Apicomplexa relates to a large phylum of single-celled, obligate intracellular protozoan organisms that all have a parasitic lifestyle. These parasites are responsible for diseases such as toxoplasmosis, malaria, neosporosis, coccidiosis and cryptosporidiosis. They have in common a specific process of host cell invasion in several steps, resulting in the formation of a parasitophorous vacuole in which the parasite develops. Apicomplexa comprise five principal groups commonly known as “gregarines”, “haemogregarines”, “coccidian”, “malarial parasites” and “piroplasms”. Parasites within the coccidian group can be either monoxenous, parasitising a single host throughout their lifecycle, or heteroxenous whereby the parasite will parasite multiple hosts.

In a specific aspect of the invention the strain of Apicomplexa is either Toxoplasma gondii and Neospora caninum, two heteroxenic coccidian parasites of the family Sarcocystidae.

In a specific aspect of the invention the strain of Apicomplexa is Toxoplasma gondii.

Toxoplasma gondii is an obligate intracellular parasite that can infect and replicate within virtually any nucleated mammalian cell. Moreover, extracellular Toxoplasma gondii can traverse the blood brain barrier and reach the central nervous system (CNS).Several strains of Toxoplasma gondii have been described and are well known in the art. Examples of strains of Toxoplasma gondii that may be used in the present invention include, but are not limited to a virulent Toxoplasma gondii type I strain (e.g., strain RH and GT-1), a Toxoplasma gondii type II strain (e.g., strain ME49), and a Toxoplasma gondii type III strain (e.g., strain VEG and CEP). In one embodiment, the strain of Toxoplasma gondii is a virulent type I strain RH. In one embodiment, the strain of Toxoplasma gondii is a virulent type II strain RH.

In a specific aspect of the invention the strain of Apicomplexa is Neospora caninum.

Neospora caninum presents the advantage of being noninfectious in human. Moreover, Neospora caninum presents no risk of encystment that could lead to unpredictable side effects after several years of treatment.

Several strains of Neospora caninum have been described and are well known in the art. Examples of strains of Neospora caninum that may be used in the present invention include, but are not limited to, Neospora caninum 1 (NC-1), Neospora caninum Liverpool, BPA1, BPA6, NC-Beef, NC-Illinois, NC-LivB1, NC-LivB2, NC-SweB1, JAP1, NC-GER1, NC-GER2, NC-GER 3, NC-GER 4, NC-GER 5, NC-GER6, NC-GER8, NC-GER9, NC-Bahia, NC-Nowra, WA-K9, NcNZ1, NcNZ2, NcNZ3 and NcIs491. In one embodiment, the strain of Neospora caninum is Neospora caninum 1 (NC-1).

In one embodiment, the strain according to the present invention is an oncolytic strain.

The term “oncolytic strain” as used herein, refers to a strain that enhances the killing of cancer cells by activating the immune system, and hence improves the cancer regression. Moreover, said strain can also directly infect, replicate in, and hence kill cancer cells. The term has used herein, encompasses the protozoan genome, the product of its expression and protozoan particles.

In a specific embodiment, the strain of the invention expresses and/or secretes one or more heterologous protein(s) selected from the group comprising therapeutic molecules, antigens, recombinant surface receptor, or combinations thereof.

In one embodiment, the strain according to the invention can be used to express any heterologous protein one would want to express within a mammalian host cell.

In one embodiment, the heterologous protein is expressed by the strain and either secreted into the parasite vacuole or secreted into the cytosol of the mammalian host cell. In one embodiment, the heterologous protein is expressed by the strain and either secreted into the extracellular compartment of the mammalian host cell. In another embodiment, the heterologous protein is expressed at the cell surface of the strain. In another embodiment, the heterologous protein is intracellularly expressed by the parasite, whatever the subcellular location within the parasite (cytoplasm, endoplasmic reticulum, mitochondrion, inner membrane complex . . . ).

Non-limiting examples of heterologous proteins that can be expressed and/or secreted by the strain include, therapeutic proteins, antigens, recombinant surface receptor and other.

By “therapeutic protein or molecule” is meant a peptide, a polypeptide, a protein or a molecule that is capable of providing a biological activity when administered appropriately to a subject, which is expected to cause a beneficial effect on the course or a symptom of the pathological condition to be treated. A vast number of therapeutic genes may be envisaged in the context of the invention such as those encoding therapeutic molecules that can induce cancer cell apoptosis, induce tumor necrosis and/or stimulate immune response against cancer cells or infected cells. They may be native genes or genes obtained from the latter by mutation, deletion, substitution and/or addition of one or more nucleotides.

Non-limiting examples of therapeutic proteins or molecules include, immunostimulatory proteins, immune checkpoint inhibitor proteins, immune checkpoint agonist proteins, angiogenesis inhibitor proteins, drugs and other molecules of therapeutic interest.

Without wishing to be bound by a theory, the strain of the invention is used for creating the microenvironment necessary for the at least one heterologous protein to be fully active. One example is the capacity of the strain of the invention to lyse the infected cells or the cancer cells and reveal targets (such as molecules, proteins, antigens) that were inaccessible to the heterologous protein. Another example is the capacity of the strain of the invention to reprogram the microenvironment via for example the secretion or expression of at least one heterologous protein (such as cytokines or surface receptor molecules . . . ) to a microenvironment more active for recruiting immune cells at the site of a tumor. Another example is the capacity of the strain of the invention to reactivate the immunosuppressive immune cells. Another example is the capacity of the strain of the invention to activate the systemic immune system, establishing a protective anti-tumor response dependent of NK cells, CD8-T cells and associated with a strong IFN-γ secretion in the tumor micro environment. In one embodiment, the therapeutic molecule is an immunostimulatory protein.

As used herein, the term “immunostimulatory protein” refers to a protein which has the ability to stimulate the immune system, in a specific or non-specific way. A vast number of proteins are known in the art for their ability to exert an immunostimulatory effect. Examples of suitable immunostimulatory proteins in the context of the invention include, without limitation, agents such as, for example, tumor necrosis factor receptor superfamily (TNFRSF) ligands, cytokines (e.g., chemokine, interleukin and tumor necrosis factor), agents that affect the regulation of cell surface receptors (e.g., inhibitors of Epidermal Growth Factor Receptor), agents that affect angiogenesis (e.g., inhibitor of Vascular Endothelial Growth Factor or inhibitors of Human Epidermal Growth Factor Receptor-2), agents that affect angiogenesis (e.g., inhibitor of Vascular Endothelial Growth Factor); agents that stimulates stem cells to produce granulocytes and macrophages (e.g., granulocyte macrophage—colony stimulating factor (GM-CSF)).

In one embodiment, the immunostimulatory protein is a human immunostimulatory protein.

In one embodiment, the immunostimulatory protein is a TNFRSF ligand selected from the group comprising: 4-1BB ligand, APRIL, BAFF, CAMLG, BAFF, CD153, CD154, CD70, Siva, EDA-A2, FasL, LIGHT, TL1A, GITR ligand, Lymphotoxin β, NGF, BDNF, NT-3, NT-4, OX40L, RANKL, TNF-α, TRAIL and TWEAK.

In one embodiment, the immunostimulatory protein is a cytokine selected from the group comprising chemokines (such as, e.g., CCL1, CCL2/MCP1, CCL3/MIP1α, CCL4/MIP1β, CCL5/RANTES, CCL6, CCL7, CCL8, CCL9, CCL11, CCL12, CCL13, CCL14, CCL15, CCL16, CCL17, CCL18/PARC/DCCK1/AMAC1/MIP4, CCL19, CCL20, CCL21, CCL22, CCL23, CCL24, CCL25, CCL26, CCL27, CCL28, CXCL1/KC, CXCL2, CXCL3, CXCL4, CXCL5, CXCL6, CXCL7, CXCL8/IL8, CXCL9, CXCL10, CXCL11, CXCL12, CXCL13, CXCL14, CXCL15, CXCL16, CXCL17, CX3CL1, XCL1 and XCL2), tumor necrosis factors (such as, e.g., TNFA, Lymphotoxin, TNFSF4, TNFSF5/CD40LG, TNFSF6, TNFSF7, TNFSF8, TNFSF9, TNFSF10, TNFSF11, TNFSF13, TNFSF13B and EDA), interleukins (such as, e.g., IL-la, IL-1β, IL-1Ra, IL-2, IL-3, IL-4, IL-5, IL-6, IL-7, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36α, IL-36β, IL-36γ, IL-36Ra, IL-37, IL-38, IFNα, IFNβ, IFNκ, IFNω and colony-stimulating factors (such as, e.g., GM-CSF, C-CSF, M-CSF).

In one embodiment, the immunostimulatory protein is a human cytokine.

In a specific embodiment, the immunostimulatory protein is human IL-15/IL-15Rα sushi (IL-15hRec).

In one embodiment, the immunostimulatory protein of the invention comprises or consists in the acid nucleic sequence of a human IL-15/IL-15Rα sushi (SEQ ID NO: 1) or an acid nucleic sequence with at least about 70%, 75%, 80%, 85%, 90%, 91%, 92%, 93%, 94%, 95%, 96%, 97%, 98% or 99% identity to SEQ ID NO: 1. In one embodiment, the immunostimulatory protein of the invention comprises or consists in the amino acid sequence of a human IL-15/IL-15Rα sushi (SEQ ID NO: 2) or an amino acid sequence with at least about 70%, 75%, 80%, 85%, 90%, 91%, 92%, 93%, 94%, 95%, 96%, 97%, 98% or 99% identity to SEQ ID NO: 2.

In one embodiment, the human IL-15/IL-15Rα sushi comprises an human interleukin-15 receptor alpha chain precursor (IL15Rα) domain having a sequence SEQ ID NO: 3 or 4, or a sequence having at least about 70% identity with SEQ ID: 3 or 4; a linker domain having a sequence SEQ ID NO: 7 or 8, or a sequence having at least about 70% identity with SEQ ID: 7 or 8; and a human interleukin-15 (IL-15) domain having a sequence SEQ ID NO: 5 or 6, or a sequence having at least about 70% identity with SEQ ID: 5 or 6.

In one embodiment, the human IL-15/IL-15Rα sushi comprises a signal domain of SUB1 having a sequence SEQ ID NO: 9 or a sequence having at least about 70% identity with SEQ ID: 9, a propeptide domain of SUB1 having a sequence SEQ ID NO: 11 or a sequence having at least about 70% identity with SEQ ID: 11, an human interleukin-15 receptor alpha chain precursor (IL15Rα) domain having a sequence SEQ ID NO: 3 or a sequence having at least about 70% identity with SEQ ID: 3, a linker domain having a sequence SEQ ID NO: 7 or a sequence having at least about 70% identity with SEQ ID: 7, and a human interleukin-15 (IL-15) domain having a sequence SEQ ID NO: 5 or a sequence having at least about 70% identity with SEQ ID: 5.

In one embodiment, the human IL-15/IL-15Rα sushi comprises a signal domain of MICS having a sequence SEQ ID NO: 13 or a sequence having at least about 70% identity with SEQ ID: 13, a propeptide domain of MICS having a sequence SEQ ID NO: 15 or a sequence having at least about 70% identity with SEQ ID: 15, an human interleukin-15 receptor alpha chain precursor (IL15Rα) domain having a sequence SEQ ID NO: 3 or a sequence having at least about 70% identity with SEQ ID: 3, a linker domain having a sequence SEQ ID NO: 7 or a sequence having at least about 70% identity with SEQ ID: 7, and a human interleukin-15 (IL-15) domain having a sequence SEQ ID NO: 5 or a sequence having at least about 70% identity with SEQ ID: 5.

In a specific embodiment, the cytokine is IL-12.

In one embodiment, the therapeutic protein is an immune checkpoint inhibitor protein.

As used herein, the term “immune checkpoint inhibitor protein” refers to a protein able to antagonize the inhibition of the immune response by an immune checkpoint. The term encompasses, without limitation, soluble domain of natural receptor, antibodies, antibody mimetics and antisense nucleic acids. In one embodiment, the protein having an immune checkpoint inhibitory activity antagonizes, at least partially, the activity of inhibitory immune checkpoints. Examples of inhibitory immune checkpoint include, but are not limited to, programmed death-1 (PD-1), programmed death ligand-1 (PD-L1), programmed death ligand-2 (PD-L2), lymphocyte-activation gene 3 (LAGS), T-cell immunoglobulin and mucin-domain containing protein 3 (TIM-3), B- and T-lymphocyte attenuator (BTLA), cytotoxic T-lymphocyte-associated protein 4 (CTLA4), T cell immunoreceptor with Ig and ITIM domains (TIGIT) and carcinoembryonic antigen-related cell adhesion molecule 1 (CEACAM-1). Examples include atezolizumab, Avelumab, Durvalumab, Nivolumab, Pembrolizumab, Cemiplimab, Spartalizumab or Ipilimumab.

In one embodiment, the therapeutic protein is an immune checkpoint agonist protein.

As used herein, the term “immune checkpoint agonist protein” refers to a protein able to agonize the immune response by an immune checkpoint. The term encompasses, without limitation, soluble domain of natural receptor, antibodies, antibody mimetics and antisense nucleic acids. In one embodiment, the protein having an agonist immune checkpoint activity agonizes, at least partially, the activity of immune checkpoints. Examples of immune checkpoint agonists include, but are not limited to, agonists of CD137 and agonists of OX40.

In one embodiment, the therapeutic protein is an angiogenesis inhibitor protein.

In a specific embodiment, the angiogenesis inhibitor protein is an inhibitor of Vascular Endothelial Growth Factor (VEGF).

In one embodiment, the therapeutic protein is a drug.

In a specific embodiment, the therapeutic protein is a chemotherapeutic drug. In another specific embodiment, the therapeutic protein is an anti-infective drug.

In one embodiment, the heterologous nucleic acid encodes one or more antigens.

Accordingly, in one embodiment, the strain according to the invention expresses one or more antigens. Preferred antigens for use herein are cancer antigens and antigens of pathogens.

In one embodiment, the antigen is an antigenic peptide.

In one embodiment, the antigen is a cancer antigen.

As used herein, the term “cancer antigen” or “tumor-associated antigen” refers to cancer antigen(s) associated with and/or serve as markers for cancers. Cancer antigens encompass various categories of peptides, e.g. those which are normally silent (i.e., not expressed) in normal cells, those that are expressed only at low levels or at certain stages of differentiation and those that are temporally expressed such as embryonic and foetal antigens as well as those resulting from mutation of cellular genes, such as oncogenes (e.g., activated ras oncogene), proto-oncogenes (e.g., ErbB family), or proteins resulting from chromosomal translocations. The cancer antigens also encompass MHC-binding cancer antigens. The cancer antigens also encompass antigens encoded by pathogenic organisms (e.g., bacteria, viruses, parasites, fungi, viroids or prions) that are capable of inducing a malignant condition in a subject (especially chronically infected subject) such as RNA and DNA tumor viruses (e.g., HPV, HCV, EBV, etc.) and bacteria (e.g., Helicobacter pilori).

Some non-limiting examples of cancer antigens include, without limitation, MART-1/Melan-A, gplOO, Dipeptidyl peptidase IV (DPPIV), adenosine deaminase-binding protein (ADAbp), cyclophilin b, Colorectal associated antigen (CRC)-C017-1A/GA733, Carcinoembryonic Antigen (CEA) and its immunogenic epitopes CAP-1 and CAP-2, etv6, am11, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family (e.g. MUC1, MUC16, etc.; see e.g. U.S. Pat. No. 6,054,438; WO98/04727; or WO98/37095), HER2/neu, p21ras, RCAS1, alpha-fetoprotein, E-cadherin, alpha-catenin, beta-catenin and gamma-catenin, p120ctn, gp100.sup.Pme1117, PRAME, NY-ESO-1, cdc27, adenomatous polyposis coli protein (APC), fodrin, Connexin 37, Ig-idiotype, p15, gp75, GM2 and GD2 gangliosides, Smad family of cancer antigens brain glycogen phosphorylase, SSX-1, SSX-2 (HOM-MEL-40), SSX-1, SSX-4, SSX-5, SCP-1 and CT-7, and c-erbB-2 and viral antigens such as the HPV-16 and HPV-18 E6 and E7 antigens and the EBV-encoded nuclear antigen (EBNA)-1.

In a particular embodiment, the antigen is a neoantigen.

As used herein, the term “neoantigen” refers to an antigen derived from proteins that result from somatic mutations or gene rearrangements acquired by a tumor. Neoantigens may be specific to each individual patient and thus provide targets for developing personalized immunotherapies. Examples of neoantigens associated with glioblastoma include, but are not limited to, the EGFR (epidermal growth factor receptor) mutant (EGFRvIII), and the IDH1 (isocitrate dehydrogenase 1) mutant. Examples of neoantigens associated with ovarian cancers include, but are not limited to, the MUC-1 mutant, the TACSTD2 (tumor associated calcium signal transducer 2) mutant, the CD318 mutant, the CD104 mutant, the N-cadherin, or the EpCAM (epithelial cell adhesion molecule) mutant. Examples of neoantigens associated with pancreatic cancers include, but are not limited to, the HSP70 mutant, the mHSP70 mutant, the MUC-1 mutant, the TACSTD2 mutant, the CEA (carcinoembryonic antigen) mutant, the CD104 mutant, the CD318 mutant, the N-cadherin mutant, or the EpCAM1 mutant. Examples of neoantigens associated with lung cancers include, but are not limited to, mutants of EGFR, KRAS, HER2, ALK, ROS1, MET, BRAF, RET or of a member of the NTRK family Examples of neoantigen associated with melanoma cancer cell include, but are not limited to, the melanocyte differentiation antigens, oncofetal antigens, tumor specific antigens, SEREX antigens or a combination thereof. Examples of melanocyte differentiation antigens, include but are not limited to tyrosinase, gp75, gplOO, MART 1 or TRP-2. Examples of oncofetal antigens include antigens in the MAGE family (MAGE-A1, MAGE-A4), BAGE family, GAGE family or NY-ESO1. Examples of tumor-specific antigens include CDK4 and 13-catenin. Examples of SEREX antigens include D-1 and SSX-2.

In a particular embodiment, the antigen is a patient-specific neoantigen.

As used herein, a “patient-specific neoantigen” is a neoantigen de novo identified in a tumor sample provided from said patient.

Examples of neoantigen expressed at the surface of cancer cells include, but are not limited to, ART4, Bcr-abl, calcium-activated chloride channel 2, CEA (carcinoembryonic antigen), EBV (Epstein-Barr virus) associated antigens (such as LMP-1, LMP-2), EpCAM, EphA3, fibronectin, Gp100/pme117, Her2/neu, immature laminin receptor, MC1R, mesothelin, MUC1, MUC2, PRAME, prostate-specific antigen (PSA), PSMA, Ras, SART-2, TGF-βRII, TRP-1/-2, tyrosinase, CD30, antigens of the BAGE family, antigens of the CAGE family, antigens of the GAGE family, antigens of the MAGE family, antigens of the SAGE family, and antigens of the XAGE family.

Techniques to identify neoantigens and patient-specific neoantigens are well-known in the art and include, without limitation, tumor sequencing, tumor transcriptional profiling (e.g., quantitative PCR, DNA microarrays, RNAseq), proteomics (e.g., 2D gel electrophoresis, 2D liquid chromatography, protein arrays, ion mobility structural analyses, glycoproteomics, glycopeptidomics, liquid chromatography-mass spectrometry, matrix assisted laser desorption ionisation-time of flight (MALDI-TOF), N-glycopeptide spectra analysis or fucosylation analysis) and metabolomics (e.g., mass spectrometry, liquid chromatography, Raman spectroscopy, emission spectroscopy, absorbance spectroscopy, circular dichroism spectroscopy, nuclear magnetic resonance (NMR), thin-layer chromatography, affinity chromatography, gas chromatography, size-exclusion chromatography and combinations thereof).

In one embodiment, the antigen is a pathogen-specific antigen.

As used herein, the term “pathogen-specific antigen” or “antigen of pathogen” refers to any antigen being fragments of infectious agent, or infectious antigens or being recombinant infectious antigens. For example, the pathogen-specific antigen can be any protein or fragment thereof selected from the group consisting of a virus, bacterium, prion, fungus, protozoon, viroid, and parasite. Pathogen-specific antigens can be derived from any human or animal pathogen.

In some embodiments, the pathogen-specific antigen is a viral antigen. The viral antigen can be from any virus that is known to be pathogenic, or against which it is desirable to elicit an immune response. In some embodiment, the viral antigen is an antigen from HIV. Specific antigens from HIV are well known in the art. Thus, a suitable antigen can be selected by one of ordinary skill in the art. For example, the HIV virus antigen can be a structural proteins (Gag, MA, CA, SP1, NC, SP2, P6, gp160, gp120, and gp140), an essential enzyme (Pol, polymerase), a gene regulatory protein (Tat and Rev); an accessory protein (Nef, Vpr, Vif and Vpu); a capsid protein, a nucleocapsid protein, a p24 viral protein, or an epitope or antigenic fragment thereof.

In some embodiments, the pathogen-specific antigen is a bacterial antigen. The bacterial antigen can be from any type of bacteria that is known to be pathogenic, or against which it is desirable to elicit an immune response. In some embodiments, the bacterial antigen is from Mycobacterium tuberculosis, the causative agent of Tuberculosis.

In some embodiments, the pathogen-specific antigen is a fungal antigen. The fungal antigen can be from any fungus that is known to be pathogenic, or against which it is desirable to elicit an immune response.

In some embodiments, the pathogen-specific antigen is a protozoan antigen. The protozoan antigen can be from any protozoan that is known to be pathogenic, or against which it is desirable to elicit an immune response. In some examples, the protozoan antigen is an antigen from a Plasmodium species, such as Plasmodium falciparum, Plasmodium vivax, Plasmodium ovale or Plasmodium malariae. Specific antigens from Plasmodium species are well known in the art. Thus, a suitable antigen can be selected by one of ordinary skill in the art. For example, the antigen from Plasmodium can be a pre-erythrocytic antigen, such as CSP or SSP2, or an erythrocytic antigen, such as AMA1 or MSP1.

In one embodiment, the heterologous nucleic acid encodes one or more recombinant surface receptor.

Accordingly, in one embodiment, the strain according to the invention expresses one or more recombinant surface receptor(s).

Techniques to express a recombinant surface receptor surface receptor in a protozoa are well-known in the art. Such techniques include, but are not limited to, the mutation of the gene coding a cell surface protein of the protozoa using molecular biology techniques well known in the art (such as, without limitation, endonuclease gene editing using, e.g., CRISPR/Cas9, TALENs or ZFNs; primer complementation; digestion with restriction enzyme and integration of a synthetic gene) to replace part or the complete gene sequence by a targeting sequence (for example, but without limitation, keeping the transmembrane domain of the cell surface protein and replacing the extracellular domain by another extracellular-binding domain, or keeping the transmembrane domain of the cell surface protein and merger the extracellular domain with another extracellular-binding domain of interest) and its integration by electroporation or chemoporation in a cell culture, which will produce the recombinant protozoa. In one embodiment, the recombinant surface receptor comprises at least one transmembrane domain anchoring said recombinant surface receptor in the cell surface of the protozoa; and at least one extracellular-binding domain.

In one embodiment, the at least one transmembrane domain may be selected from any transmembrane domain of a protozoa protein. In one embodiment, transmembrane domains suitable for anchoring a recombinant surface receptor in the cell surface of the strain of the invention are well known from the one skilled in the art. For the sake of exemplary purposes, a list of suitable transmembrane domains is given hereafter.

In one embodiment, the at least one transmembrane domain may be selected from the group comprising, but not limited to, glycosylphosphatidylinositol (GPI)-anchoring domains structurally related to the surface antigen SAG1 or to any SAG1-related sequence (SRS superfamily).

In one embodiment, the at least one transmembrane domain of the invention comprises or consists in the acid nucleic sequence of the GPI-anchoring domain of the major surface antigen SAG-1 of Toxoplasma gondii (SEQ ID NO: 17) or an acid nucleic sequence with at least about 70%, 75%, 80%, 85%, 90%, 91%, 92%, 93%, 94%, 95%, 96%, 97%, 98% or 99% identity to SEQ ID NO: 17. In one embodiment, the at least one GPI-anchoring domain of the invention comprises or consists in the amino acid sequence of the GPI-anchoring domain of the major surface antigen SAG-1 of Toxoplasma gondii (SEQ ID NO: 18) or an amino acid sequence with at least about 70%, 75%, 80%, 85%, 90%, 91%, 92%, 93%, 94%, 95%, 96%, 97%, 98% or 99% identity to SEQ ID NO: 18.

The one skilled in the art is familiar with techniques allowing the selection of an extracellular-binding domain known in the art to specifically bind a desired target. For the sake of exemplary purposes, a list of suitable extracellular-binding domains is given hereafter.

In one embodiment, the at least one extracellular-binding domain is an antigen-binding domain.

In one embodiment, the antigen-binding domain is an antibody or antigen-binding fragment thereof. The portion of the recombinant surface receptor of the invention comprising an antibody or antigen-binding fragment thereof may exist in a variety of forms where the ligand binding domain is expressed as part of a contiguous polypeptide chain including, for example, a single domain antibody fragment (sdAb), a single chain antibody (scFv), a humanized antibody or bispecific antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a chimeric receptor composition of the invention comprises an antibody fragment. In a further aspect, the chimeric receptor comprises an antibody fragment that comprises a scFv. The precise amino acid sequence boundaries of a given CDR can be determined using any of a number of well-known schemes, including those described by Kabat et al. (1991), “Sequences of Proteins of Immunological Interest”, 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof.

In one embodiment, said antibody is an antibody molecule selected from the group consisting of a whole antibody, a humanized antibody, a single chain antibody, a dimeric single chain antibody, a Fv, a scFv, a Fab, a F(ab)′2, a defucosylated antibody, a bi-specific antibody, a diabody, a triabody, a tetrabody.

In another embodiment, said antibody is an antibody fragment selected from the group consisting of a unibody, a domain antibody, and a nanobody.

In another embodiment, said antibody is an antibody mimetic selected from the group consisting of an affibody, an alphabody, an armadillo repeat protein-based scaffold, a knottin, a kunitz domain peptide, an affilin, an affitin, an adnectin, an atrimer, an evasin, a DARPin, an anticalin, an avimer, a fynomer, a versabody and a duocalin.

Fragments and derivatives of antibodies of this invention (which are encompassed by the term “antibody” as used in this application, unless otherwise stated or clearly contradicted by context), can be produced by techniques that are known in the art. “Fragments” comprise a portion of the intact antibody, generally the antigen binding site or variable region. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods.

For instance, Fab or F(ab′)₂ fragments may be produced by protease digestion of the isolated antibodies, according to conventional techniques. It will be appreciated that immunoreactive fragments can be modified using known methods, for example to slow clearance in vivo and obtain a more desirable pharmacokinetic profile the fragment may be modified with polyethylene glycol (PEG). Methods for coupling and site-specifically conjugating PEG to a Fab′ fragment are described in, for example, Leong et al., Cytokines 16 (3): 106-119 (2001) and Delgado et al., Br. J. Cancer 5 73 (2): 175-182 (1996), the disclosures of which are incorporated herein by reference.

In one embodiment, the antigen-binding domain of the recombinant surface receptor comprises or consists in an antibody fragment, such as, for example, a scFv.

In one embodiment, the antigen-binding domain is a scFv.

In one embodiment, the antigen-binding domain of the recombinant surface receptor recognizes a specific antigen or fragments thereof.

In another embodiment, the antigen-binding domain of the recombinant surface receptor recognizes a specific antigen or fragments thereof that are associated with a target cell.

Thus, the antigen-binding domain of the recombinant surface receptor may recognize target cells such as for example, infected cells, damaged cells, or dysfunctional cells. Examples of such target cells may include cells involved in dysfunctional immune reactions, dysfunctionally activated inflammatory cells, cancer cells, virally infected cells and bacterially infected cells. In one embodiment, the antigen-binding domain of the recombinant surface receptor recognizes a specific antigen or fragments thereof that are associated with a cancer cell.

In a specific embodiment, the antigen-binding domain of the recombinant surface receptor is specific of cancer antigen or fragments thereof.

In a specific embodiment, the antigen-binding domain of the recombinant surface receptor is specific of solid cancer antigen or fragments thereof. In a specific embodiment, the antigen-binding domain of the recombinant surface receptor is specific of ovarian cancer antigen or fragments thereof. In a specific embodiment, the antigen-binding domain of the recombinant surface receptor is specific of pancreatic cancer antigen or fragments thereof. In a specific embodiment, the antigen-binding domain of the recombinant surface receptor is specific of lung cancer antigen or fragments thereof. In a specific embodiment, the antigen-binding domain of the recombinant surface receptor is specific of melanoma antigen or fragments thereof. In a specific embodiment, the antigen-binding domain of the recombinant surface receptor is specific of glioblastoma antigen or fragments thereof, such as, for example, OGD2.

In another embodiment, the antigen-binding domain of the recombinant surface receptor recognizes a specific antigen or fragments thereof that are associated with an immune cell.

In another embodiment, the antigen-binding domain of the recombinant surface receptor may recognize antigen-presenting cells.

As used herein, the terms “antigen-presenting cells” or “APCs” are used to refer to autologous cells that express MHC Class I and/or Class II molecules that present antigens to T cells. Examples of antigen-presenting cells include, e.g., professional or non-professional antigen processing and presenting cells. Examples of professional APCs include, e.g., B cells, whole spleen cells, monocytes, macrophages, dendritic cells, fibroblasts or non-fractionated peripheral blood mononuclear cells (PMBC). Examples of hematopoietic APCs include dendritic cells, B cells and macrophages. Of course, it is understood that one of skill in the art will recognize that other antigen-presenting cells may be useful in the invention and that the invention is not limited to the exemplary cell types described herein.

In one embodiment, said scFv is directed to antigen-presenting cells. In one embodiment, said scFv is directed to dendritic cells.

In one embodiment, said scFv is directed to an endocytic receptor.

For example, in one embodiment, the strain according to the invention expresses a recombinant surface receptor that can specifically binds to an endocytic receptor, such as, for example, DEC-205 (CD205). In one embodiment, the strain expressing a recombinant surface receptor can specifically binds to an endocytic receptor, such as, for example, DEC-205 (CD205), and therefore can be endocyted by the cell expressing said endocytic receptor.

In one embodiment, said scFv is directed to DEC-205. Example of scFv directed to dec-205 include, but is not limited to, SEQ ID NO: 19 and/or 20 or a sequence with at least about 70%, 75%, 80%, 85%, 90%, 91%, 92%, 93%, 94%, 95%, 96%, 97%, 98% or 99% identity to SEQ ID NO: 19 and/or 18

A used herein, “DEC205” or “CD205” refers to an endocytic receptor that is expressed at high levels by dendritic cell (DC) subsets, including the DC population that is responsible for cross-presentation of antigens.

In one embodiment, the at least one extracellular-binding domain of the invention comprises or consists in the acid nucleic sequence of a scFv directed to DEC-205 (SEQ ID NO: 19) or an acid nucleic sequence with at least about 70%, 75%, 80%, 85%, 90%, 91%, 92%, 93%, 94%, 95%, 96%, 97%, 98% or 99% identity to SEQ ID NO: 19. In one embodiment, the at least one extracellular-binding domain of the invention comprises or consists in the amino acid sequence of a scFv directed to DEC-205 (SEQ ID NO: 20) or an amino acid sequence with at least about 70%, 75%, 80%, 85%, 90%, 91%, 92%, 93%, 94%, 95%, 96%, 97%, 98% or 99% identity to SEQ ID NO: 20.

In one embodiment, the at least one extracellular-binding domain of the invention comprises a N terminal signal sequence of SAG1 (SEQ ID NO: 21) or an acid nucleic sequence with at least about 70%, 75%, 80%, 85%, 90%, 91%, 92%, 93%, 94%, 95%, 96%, 97%, 98% or 99% identity to SEQ ID NO: 21. In one embodiment, the at least one extracellular-binding domain of the invention comprises a N terminal signal sequence of SAG1 (SEQ ID NO: 22) or an amino acid sequence with at least about 70%, 75%, 80%, 85%, 90%, 91%, 92%, 93%, 94%, 95%, 96%, 97%, 98% or 99% identity to SEQ ID NO: 22.

In one embodiment, the at least one extracellular-binding domain of the invention may further comprise a truncated SAG1 (SEQ ID NO: 23) or an acid nucleic sequence with at least about 70%, 75%, 80%, 85%, 90%, 91%, 92%, 93%, 94%, 95%, 96%, 97%, 98% or 99% identity to SEQ ID NO: 21. In one embodiment, the at least one extracellular-binding domain of the invention may further comprise a truncated SAG1 (SEQ ID NO: 24) or an amino acid sequence with at least about 70%, 75%, 80%, 85%, 90%, 91%, 92%, 93%, 94%, 95%, 96%, 97%, 98% or 99% identity to SEQ ID NO: 24.

Optionally, a tag may be comprised into the recombinant surface receptor sequence. In one embodiment, a HA tag (SEQ ID NO: 25 or 26) is used as a suitable tag.

Optionally, a Kozak consensus sequence may be comprised into the recombinant surface receptor sequence to initiate the translation process of the recombinant receptor.

Optionally, a signal peptide may be comprised into the recombinant surface receptor sequence. In one embodiment, a SS sequence is used as a suitable signal peptide.

Optionally, a short oligo- or polypeptide linker, for example, between 2 and 10 amino acids (e.g., 2, 3, 4, 5, 6, 7, 8, 9, or 10 amino acids) in length may form the linkage between distinct domains of the recombinant receptor. In one embodiment, a GGGAS (SEQ ID NO: 27 or 28) is used as a suitable linker.

In one embodiment, the recombinant surface receptor of the invention comprises or consists in the amino acid sequence of the transmembrane domain (SEQ ID NO: 18) or an amino acid sequence with at least about 70%, 75%, 80%, 85%, 90%, 91%, 92%, 93%, 94%, 95%, 96%, 97%, 98% or 99% identity to SEQ ID NO: 18 and the amino acid sequence of the extracellular-binding domain (SEQ ID NO: 29) or an amino acid sequence with at least about 70%, 75%, 80%, 85%, 90%, 91%, 92%, 93%, 94%, 95%, 96%, 97%, 98% or 99% identity to SEQ ID NO: 29.

In one embodiment, the recombinant surface receptor of the invention comprises or consists in the amino acid sequence of the transmembrane domain (SEQ ID NO: 18) or an amino acid sequence with at least about 70%, 75%, 80%, 85%, 90%, 91%, 92%, 93%, 94%, 95%, 96%, 97%, 98% or 99% identity to SEQ ID NO: 18 and the amino acid sequence of the extracellular-binding domain (SEQ ID NO: 30) or an amino acid sequence with at least about 70%, 75%, 80%, 85%, 90%, 91%, 92%, 93%, 94%, 95%, 96%, 97%, 98% or 99% identity to SEQ ID NO: 30.

In one embodiment, the recombinant surface receptor of the invention comprises or consists in the amino acid sequence SEQ ID NO: 31 or an acid nucleic sequence with at least about 70%, 75%, 80%, 85%, 90%, 91%, 92%, 93%, 94%, 95%, 96%, 97%, 98% or 99% identity to SEQ ID NO: 31.

In one embodiment, the recombinant surface receptor of the invention comprises or consists in the acid nucleic sequence SEQ ID NO: 32 or an acid nucleic sequence with at least about 70%, 75%, 80%, 85%, 90%, 91%, 92%, 93%, 94%, 95%, 96%, 97%, 98% or 99% identity to SEQ ID NO: 32.

In one embodiment, the strain according to the invention can accommodate multiple expression constructs. Therefore, several heterologous nucleic acid molecules encoding several heterologous proteins can be integrated into the strain genome.

Accordingly, in one embodiment, the strain according to the invention expresses at least one heterologous protein selected from the groups of, immunostimulatory proteins, antigens and at least one recombinant receptors.

In one embodiment, the strain according to the invention expresses at least one immunostimulatory protein. In one embodiment, the strain according to the invention expresses at least one antigen. In one embodiment, the strain according to the invention expresses at least one recombinant receptor.

In one embodiment, the strain according to the invention expresses at least one immunostimulatory protein and at least one antigen. In one embodiment, the strain according to the invention expresses at least one immunostimulatory protein and at least one recombinant receptor. In one embodiment, the strain according to the invention expresses at least one antigen protein and at least one recombinant receptor.

In one embodiment, the strain according to the invention expresses at least one immunostimulatory protein, at least one antigen, and at least one recombinant receptor.

Examples of a strain accommodating multiple constructs are strains expressing/secreting an antigen and/or a cytokine such as for example IL-15hRec and/or an antibody such as an anti-PD1 antibody or an anti-PDL1 antibody.

In one embodiment, the strain as described hereinabove is a tachyzoite.

By “tachyzoite” is meant the rapidly multiplying form of the strain of the invention (e.g., Toxoplasma gondii and Neospora caninum). The tachyzoite has usually a crescent shape and a variable size, for example a size of about 5-8×2-3 μm. The apical part of the parasite comprises conoids which participate in the penetration of the parasite into the host cell. The micronemes, the rhoptries and the dense granules constitute the three major organelles of the tachyzoite, which also comprises a nucleus, an apicoplast, a Golgi apparatus, an endoplasmic reticulum and an organite similar to the mitochondrion.

In one embodiment, the strain of Toxoplasma gondii is a tachyzoite from Toxoplasma gondii RH. In one embodiment, the strain of Toxoplasma gondii is a tachyzoite from Toxoplasma gondii ME49.

In one embodiment, the strain of Neospora caninum is a tachyzoite from Neospora caninum-1 (NC-1).

Methods for obtaining and maintaining living strains, in particular tachyzoites, of Toxoplasma gondii and Neospora caninum are well known by the skilled artisan. Example of a method for maintaining strains of Toxoplasma gondii and Neospora caninum is indicated below.

Human foreskin fibroblast (HFF) cells are widely used to culture and maintain tachyzoites. HFF cells can be cryopreserved in 95% culture medium with 5% DMSO in liquid nitrogen or −150° C. freezer for many years. Viability of the HFF cells after cryopreservation depends on several factors, including the stresses imposed on HFF cells during the freezing and thawing procedure. Tachyzoites of the Toxoplasma gondii or Neospora caninum strain are harvested from infected HFF. Briefly, HFF are cultured in monolayers in DMEM, supplemented with 10% heat-inactivated FCS, 50 U/ml penicillin/50 μg/ml streptomycin, and 1% HEPES at 37° C. in 5% CO2 atmosphere. Finally, tachyzoites may be harvested when monolayers of HFF are completely lysed.

In one embodiment, the strain as described hereinabove is substantially purified. In one embodiment, the strain as described hereinabove is isolated.

The strain of the invention may also be used as an external bioreactor for producing an heterologous protein.

Thus, another object of the invention is a method of producing at least one heterologous protein by a strain of the invention, said method comprising:

a) infecting a cell with a strain of the invention, wherein the strain secretes said at least one heterologous protein,

b) cultivating the infected cell in a culture medium,

c) recovering the least one heterologous protein.

The heterologous protein can be recovered in the culture medium and/or directly within the cells by methods known in the art.

The cell used for infection by the strain of the invention can be any cell or cell line adapted for producing an heterologous protein. In one embodiment, the cell is a Chinese hamster ovary (CHO) cell. In another embodiment, the cell is a Human foreskin fibroblast (HFF).

Another object of the present invention is a composition comprising, consisting or consisting essentially of at least one strain as described hereinabove.

As used herein, “consisting essentially of”, with reference to a composition, means that at least one strain of Toxoplasma gondii or at least one strain of Neospora caninum according to the invention, or combination thereof is the only one therapeutic agent or agent with a biologic activity within said composition.

Another object of the invention is a composition wherein said composition is a pharmaceutical composition comprising, consisting or consisting essentially of at least one strain as described hereinabove and at least one pharmaceutically acceptable excipient.

Examples of pharmaceutically acceptable excipients include, but are not limited to, media, solvents, coatings, isotonic and absorption delaying agents, additives, stabilizers, preservatives, surfactants, substances which inhibit enzymatic degradation, alcohols, pH controlling agents, and propellants.

Examples of pharmaceutically acceptable media include, but are not limited to, water, phosphate buffered saline, normal saline or other physiologically buffered saline, or other solvent such as glycol, glycerol, and oil such as olive oil or an injectable organic ester. A pharmaceutically acceptable medium can also contain liposomes or micelles, and can contain immunostimulating complexes prepared by mixing polypeptide or peptide antigens with detergent and a glycoside.

Examples of coating materials include, but are not limited to, lecithin.

Examples of isotonic agents include, but are not limited to, sugars, sodium chloride, and the like. Examples of agents that delay absorption include, but are not limited to, aluminum monostearate and gelatin.

Examples of additives include, but are not limited to, mannitol, dextran, sugar, glycine, lactose or polyvinylpyrrolidone or other additives such as antioxidants or inert gas, stabilizers or recombinant proteins (e.g., human serum albumin) suitable for in vivo administration.

Examples of suitable stabilizers include, but are not limited to, sucrose, gelatin, peptone, digested protein extracts such as NZ- Amine or NZ-Amine AS.

A further object of the invention is a medicament comprising, consisting or consisting essentially of at least one strain as described hereinabove.

Another object of the invention is a vaccine composition comprising, consisting or consisting essentially of at least one strain as described hereinabove.

In one embodiment, the vaccine of the invention is a prophylactic vaccine. In another embodiment, the vaccine of the invention is a therapeutic vaccine.

In one embodiment, the vaccine composition further comprises at least one adjuvant.

As used herein, the term “adjuvant” refers to a substance that enhances, augments or potentiates the host's immune response induced by the strain of the present invention.

Examples of adjuvants that can be used in the vaccine composition include, but are not limited to, ISA51; emulsions such as CFA, MF59, montanide, AS03 and AF03; mineral salts such as alum, calcium phosphate, iron salt, zirconium salt, and ASO4; TLR ligands such as TLR2 ligands (such as outer-surface protein A or OspA), TLR3 ligands (such as poly I:C), TLR4 ligands (such as MPL and GLA), TLR5 ligands, TLR7/8 ligands (such as imiquimod), TLR9 ligands (such as CpG ODN); polysacharrides such as chitin, chitosan, α-glucans, 0-glucans, fructans, mannans, dextrans, lentinans, inulin-based adjuvants (such as gamma inulin); TLR9 and STING ligands such as K3 CpG and cGAMP.

The strain, the composition, the pharmaceutical composition, the medicament or the vaccine composition of the present invention may be administered orally, parenterally, by intraperitoneal administration, by inhalation spray, topically, rectally, nasally, buccally, vaginally or via an implanted reservoir.

In one embodiment, the strain, the composition, the pharmaceutical composition, the medicament or the vaccine composition of the present invention is injected. Examples of injections include, but are not limited to, intratumoral, intradermal, subcutaneous, intravenous, intramuscular, intra-lymphatic, intra-articular, intra-synovial, intrasternal, intrathecal, intravesical, intravaginal, intrahepatic, intralesional and intracranial injection or infusion techniques.

In a further aspect of the invention, the strain, the composition or the vaccine composition is administrated via subcutaneous, intradermal, intraperitoneal, intravaginal or intratumoral routes

In one embodiment, the strain, the composition, the pharmaceutical composition, the medicament or the vaccine composition of the present invention is in a form adapted to oral administration. According to a first embodiment, the form adapted to oral administration is a solid form selected from the group comprising tablets, pills, capsules, soft gelatin capsules, sugarcoated pills, orodispersing tablets, effervescent tablets or other solids. According to a second embodiment, the form adapted to oral administration is a liquid form, such as, for example, a drinkable solution, a buccal spray, liposomal forms and the like.

In another embodiment, strain, the composition, the pharmaceutical composition, the medicament or the vaccine composition of the present invention is formulated for rectal or vaginal administration and may be presented as suppositories, pessaries, tampons, creams, gels, pastes, foams or sprays.

In another embodiment, strain, the composition, the pharmaceutical composition, the medicament or the vaccine composition of this invention is in a form suitable for parenteral administration. Forms suitable for parenteral administrations include, but are not limited to, sterile isotonic aqueous or non-aqueous solutions, dispersions, suspensions or emulsions, or sterile powders which may be reconstituted into sterile injectable solutions or dispersions just prior to use.

In another embodiment, the strain, the composition, the pharmaceutical composition, the medicament or the vaccine composition of the invention is in a form adapted for local delivery via the nasal and respiratory routes. Examples of formulations suitable for nasal or respiratory administration include, but are not limited to, nasal solutions, sprays, aerosols and inhalants.

In another embodiment, the strain, the composition, the pharmaceutical composition, the medicament or the vaccine composition of the invention is in a form adapted to a topical administration. Examples of formulations adapted to a topical administration include, but are not limited to, ointment, paste, eye drops, cream, patch, such as, for example, transdermal patch, gel, liposomal forms and the like.

In one embodiment, the composition or formulation of the invention may be presented in unit-dose or multi-dose sealed containers, for example, ampoules and vials. In one embodiment, the composition or formulation of the invention may be frozen and then stored. In another embodiment, the composition or formulation of the invention may be be stored in a lyophilized condition requiring only the addition of the sterile liquid excipient, for example water for injection, immediately prior to use. Extemporaneous injection solutions and suspensions may be prepared from sterile powders, granules and tablets.

The exact dose for administration can be determined by the skilled practitioner, in light of factors related to the subject that requires treatment. Dosage is adjusted to provide sufficient levels of the composition or to maintain the desired effect of reducing signs or symptoms of the targeted pathologic condition or disorder, or reducing severity of the targeted pathologic condition or disorder. Factors which may be taken into account include the severity of the disease state (such as for example the tumor volume or the number of infected cells), the prognosis of the disease, the localization or accessibility to the tumor, general health of the subject, age, weight, and gender of the subject, diet, time and frequency of administration, drug combination(s), reaction sensitivities, and tolerance/response to therapy.

In one embodiment, a therapeutically effective amount of the strain, the composition, the pharmaceutical composition, the medicament or the vaccine composition of the present invention is administered (or is to be administered) to the subject.

In one embodiment, the strain or the composition, the pharmaceutical composition, the medicament or the vaccine composition is administered at least once, preferably at least twice, more preferably at least three times, and even more preferably at least four times at day, week or month intervals, according to a prime/boost mode.

In one embodiment, the amount strains (preferably of tachyzoites) as described hereinabove administered to the subject is at least of 10⁴, 10⁵, 10⁶, 10⁷, 10⁸, 10⁹, 10¹⁰ or 10¹¹ strains.

In one embodiment, the amount of strains (preferably of tachyzoites) as described hereinabove administered per administration ranges from about 10⁴ to about 10¹¹, preferably from about 10⁵ to about 10¹⁰, more preferably from about 10⁶ to about 10⁹, and even more preferably from about 10⁷ to about 10⁸, including all integer values within those ranges.

In one embodiment, the daily amount of strains (preferably of tachyzoites) as described hereinabove administered per day to the subject is at least of 10⁴ per day, 10⁵ per day, 10⁶ per day, 10⁷ per day, 10⁸ per day, 10⁹ per day, 10¹⁰ per day or 10¹¹ per day of strains.

In one embodiment, the daily amount of strains (preferably of tachyzoites) as described hereinabove administered per day ranges from about 10⁴ to about 10¹¹ per day, preferably from about 10⁵ to about 10¹⁰ per day, more preferably from about 10⁶ to about 10⁹ per day, and even more preferably from about 10⁷ to about 10⁸ per day, including all integer values within those ranges.

In one embodiment, the amount of immune cells strains (preferably of tachyzoites) as described hereinabove administered to the subject is at least of 10¹, 10², 10³, 10⁴, 10⁵, 10⁶, 10⁷, 10⁸ or 10⁹ strains/kg body.

In one embodiment, the strain of the present invention is administered in combination with at least one heterologous protein as described in the specification.

Thus, another object of the invention is a combination comprising a strain of the present invention and at least one heterologous protein as described in the specification.

As described hereabove, the at least one heterologous protein to be combined with the strain of the invention is a therapeutic protein or molecule, an antigen or a recombinant surface receptor, such as for example, immunostimulatory proteins such as TFRSF ligand or cytokine, immune checkpoint inhibitor proteins, immune checkpoint agonist proteins, angiogenesis inhibitor proteins, drugs, antibodies.

Without wishing to be bound by a theory, the strain of the invention is used in the combination for creating the microenvironment necessary for the heterologous protein to be fully active. One example is the capacity of the strain of the invention to lyse the infected cells and the cancer cells and reveal targets (such as molecules, proteins, antigens) that were inaccessible to the heterologous protein. Another example is the capacity of the strain of the invention to reprogram the microenvironment via for example the secretion or expression of at least one heterologous protein (such as cytokines or surface receptor molecules . . . ) to a microenvironment more active for recruiting immune cells at the site of a tumor. Another example is the capacity of the strain of the invention to reactivate the immunosuppressive immune cells. Another example is the capacity of the strain of the invention to activate the systemic immune system, establishing a protective anti-tumor response dependent of NK cells, CD8-T cells and associated with a strong IFN-γ secretion in the tumor micro environment. The expression “combined preparation” or “combination” refers to any preparation comprising at least two components. The different components of the combined preparation, or of the combination, may be used simultaneously, semi-simultaneously, separately, sequentially or spaced out over a period of time so as to obtain the maximum efficacy of the combination.

For instance, they may be administered concurrently, i.e. simultaneously in time, or sequentially, i.e. one component is administered after the other one(s). After administration of the first component, the other component(s) can be administered substantially immediately thereafter or after an effective time period. The effective time period is the amount of time given for realization of maximum benefit from the administration of the components.

As a result, for the purposes of the present invention, the combined preparations or combinations are not limited to those which are obtained by physical association of the constituents, but may also be in the form of separate products permitting a separate administration, which can be simultaneous or spaced out over a period of time.

Alternatively, the different components may be co-formulated.

The present invention further relates to the strain, the composition, the pharmaceutical composition, the medicament or the vaccine composition, for use in preventing and/or treating cancer or a chronic infectious disease in a subject in need thereof. It also relates to methods of preventing and/or treating cancer or a chronic infectious disease, by administering to a subject in need thereof the strain, the composition, the pharmaceutical composition, the medicament or the vaccine composition according to the present invention.

In one embodiment, the strain, the composition, the pharmaceutical composition, the medicament or the vaccine composition is for treating cancer.

In one embodiment, the strain, the composition, the pharmaceutical composition, the medicament or the vaccine composition is for use in treating a tumor, preferably a solid tumor.

Indeed, the Inventors have shown that the administration of the strain according to the invention induces a decrease of the tumor volume in mice (see Examples). Therefore, in one embodiment, the strain according to the invention is for inducing a decrease of the tumor volume and/or for preventing an increase of the tumor volume in a subject in need thereof.

In one embodiment, the cancer may be any solid tumor. Examples of solid tumors include but are not limited to, bile duct cancer (e.g., periphilar cancer, distal bile duct cancer, intrahepatic bile duct cancer), bladder cancer, bone cancer (e.g., osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g., meningioma, astocytoma, oligodendrogliomas, ependymoma, gliomas, glioblastoma, medulloblastoma, ganglioglioma, Schwannoma, germinoma, craniopharyngioma), breast cancer (e.g., ductal carcinoma in situ, infiltrating ductal carcinoma, infiltrating lobular carcinoma, lobular carcinoma in, situ, gynecomastia), Castleman disease (e.g., giant lymph node hyperplasia, angio follicular lymph node hyperplasia), cervical cancer, colorectal cancer, endometrial cancer (e.g., endometrial adenocarcinoma, adenocanthoma, papillary serous adnocarcinroma, clear cell), esophagus cancer, gallbladder cancer (e.g., mucinous adenocarcinoma, small cell carcinoma), gastrointestinal carcinoid tumors (e.g., choriocarcinoma, chorioadenoma destruens), Hodgkin's disease, non-Hodgkin's lymphoma, Kaposi's sarcoma, kidney cancer (e.g., renal cell cancer), laryngeal and hypopharyngeal cancer, liver cancer (e.g., hemangioma, hepatic adenoma, focal nodular hyperplasia, hepatocellular carcinoma), lung cancer (e.g., small cell lung cancer, non-small cell lung cancer), mesothelioma, plasmacytoma, nasal cavity and paranasal sinus cancer (e.g., esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g., embryonal rhabdomyosarcoma, alveolar rhabdomyosarcoma, pleomorphic rhabdomyosarcoma), salivary gland cancer, skin cancer (e.g., melanoma, nonmelanoma skin cancer), stomach cancer, testicular cancer (e.g., seminoma, nonseminoma germ cell cancer), thymus cancer, thyroid cancer (e.g., follicular carcinoma, anaplastic carcinoma, poorly differentiated carcinoma, medullary thyroid carcinoma, thyroid lymphoma), vaginal cancer, vulvar cancer, and uterine cancer (e.g., uterine leiomyosarcoma).

In one embodiment, the cancer is selected from ovarian cancer, pancreatic cancer, lung cancer and melanoma.

In one embodiment, the cancer is glioma.

In one embodiment, the cancer is glioblastoma.

In one embodiment, the strain, the composition, the pharmaceutical composition, the medicament or the vaccine composition is for use in treating blood cancer.

In one embodiment, the strain, the composition, the pharmaceutical composition, the medicament or the vaccine composition is for use in preventing cancer.

In one embodiment, the strain, the composition, the pharmaceutical composition, the medicament or the vaccine composition is for use in treating metastatic cancer. In one embodiment, the strain, the composition, the pharmaceutical composition, the medicament or the vaccine composition is for use in preventing the occurrence of metastasis, and/or for reducing the number of metastasis in a subject in need thereof.

In one embodiment, the strain, the composition, the pharmaceutical composition, the medicament or the vaccine composition is for use in treating recurrent cancer. In one embodiment, the strain, the composition, the pharmaceutical composition, the medicament or the vaccine composition is for use in preventing the recurrence of cancer.

As used herein, the term “recurrence” or “recurrent” refers to cancer that has recurred (come back), usually after a period of time during which the cancer could not be detected. The cancer may come back to the same place as the primary tumor or to another place in the body. For example, glioblastoma is associated with high level of recurrence.

In one embodiment, the strain, the composition, the pharmaceutical composition, the medicament or the vaccine composition is for use in treating an infectious disease.

In one embodiment, said infectious disease is a chronic infectious disease, i.e., a disease due to the prolonged and persistent invasion of the body of a subject by pathogens (including, for example, parasites, bacteria (in particular mycobacteria) and viruses).

In one embodiment, said infectious disease is a chronic virus infection, such as, for example, a HIV infection.

In one embodiment, said infectious disease is a chronic bacterial infection, such as, for example, an infection by Mycobacterium tuberculosis.

In one embodiment, said infectious disease is a chronic infection with a parasite, such as, for example, an infection by plasmodium.

In one embodiment, said chronic infectious disease is caused by the presence of an intracellular pathogen, such as, for example, an intracellular virus, an intracellular bacterium or an intracellular parasite.

In one embodiment, said infectious disease is a chronic infectious disease associated with an immunodepletion or an immunosuppression or inducing an immunodepletion or an immunosuppression.

Examples of chronic infectious diseases associated with or inducing an immunodepletion or an immunosuppression include, but are not limited to, tuberculosis, HIV or malaria infections.

In one embodiment, said infectious disease is not due to an apicomplexan of the family Sarcocystidae. In one embodiment, said infectious disease is not a cryptosporidiosis. In one embodiment, said infectious disease is not toxoplasmosis or neosporosis.

In one embodiment, the subject is a human.

In one embodiment, the subject is immunosuppressed, i.e., presents an impaired immune system. In one embodiment, the immune system of the subject has compromised ability to fight a cancer or an infectious disease.

In one embodiment, the subject has cancer. In one embodiment, the subject is diagnosed or has been diagnosed with cancer.

In one embodiment, the cancer is early or late stage cancer.

In one embodiment, the subject was not treated previously with another treatment for cancer (i.e., the method of the invention is the first line treatment).

In another embodiment, the subject previously received one, two or more other treatments for cancer (i.e., the method of the invention is a second line, a third line or more). In one embodiment, the subject previously received one or more other treatments for cancer, but was unresponsive or did not respond adequately to these treatments, which means that there is no or too low therapeutic benefit induced by these treatments.

In another embodiment, the subject is at risk of developing cancer. Examples of risk factors for developing cancer include, but are not limited to, family history of cancer, genetic predisposition, or exposure to a carcinogen.

In another embodiment, the subject has an infectious disease, preferably a chronic infectious disease. In one embodiment, the subject has an infectious disease (preferably a chronic infectious disease) associated with or inducing an immunodepletion or an immunosuppression. In one embodiment, the subject has a chronic infectious disease caused by an intracellular pathogen. In one embodiment, the subject has a tuberculosis infection. In another embodiment, the subject has an HIV infection. In another embodiment, the subject has a malaria infection.

In one embodiment, the subject was not treated previously with another treatment for the said infection (i.e., the method of the invention is the first line treatment).

In another embodiment, the subject previously received one, two or more other treatments for the said infection (i.e., the method of the invention is a second line, a third line or more). In another embodiment, the subject previously received one or more other treatments for the said infection, but was unresponsive or did not respond adequately to these treatments, which means that there is no or too low therapeutic benefit induced by these treatments.

In one embodiment, the subject is not affected by a cryptosporidiosis. In one embodiment, the subject is not affected by a toxoplasmosis or neosporosis.

### BRIEF DESCRIPTION OF THE DRAWINGS

FIG. 1 shows the construction of the RH-OVA strain with (A) the plasmid pUC 8.1 CAT/GFP-SAG1/OVA/GPI and the cell surface expression of SAG1-OVA by immunofluorescence assay (B).

FIG. 2 shows the study scheme of the treatment with RH-OVA or RH ctrl strains (A) and their effects on the tumor volume kinetic (B), the tumor volume (C), (D) Shows an image illustrating the tumor size in mice inoculated with the EG7 control and RH-OVA or RH ctrl strains, the tumor weight (E), and the tumor implantation (F) in mice inoculated or not with the EG7-OVA tumor cell line.

FIG. 3 is a group of graphs showing the effect of the treatment with RH-OVA or RH ctrl strains on cytokines: IL-2 (A), IL-5 (B), IL-4 (C), IL-10 (D), GM-CSF (E), IL-17A (F), IL-15, (G), TGF-β (H) and IFNγ (I) secretion measured by ELISA in cells derived from the spleen of mice inoculated or not with the EG7-OVA tumor cell line.

FIG. 4 is a group of graphs showing the effect of the treatment with RH-OVA strains on splenic cells populations: CD4⁺ T cells (A), CD8⁺ T cells (B), Foxp3⁺ T cells (C), NK1.1⁺ NK cells (D) and CD11c⁺ T cells (E) in mice inoculated or not with the EG7-OVA tumor cell line.

FIG. 5 is a group of graphs showing the effect of the treatment with RH-OVA or RH ctrl strains on the secretion of IFNγ of cells derived from brachial lymph node (A) and inguinal lymph node (B) in mice inoculated with the EG7-OVA tumor cell line.

FIG. 6 shows the effect of the treatment with RH-OVA or RH ctrl strains on the secretion of cytokines: IFNγ (A), IL-15 (B), IL-17A (C), IL-5 (D), IL-23 (E), TNFα (F), IL-4 (G) and TGF-β (H) of cells derived from the tumor in mice inoculated with the EG7-OVA tumor cell line.

FIG. 7 shows the effect of the treatment with RH-OVA or RH ctrl strains on cell populations: Treg cells (A), NKp46⁺ NK cells (B) and Ly6C⁺ L6G⁺ neutrophils (C) derived from the tumor in mice inoculated with the EG7-OVA tumor cell line.

FIG. 8 shows graphs of the dosage of IL12p40 (A) and IL-10 (B) secreted by immunocompetent dendritic cells (DC) and tolerogenic dendritic cells (TolDC) co-cultured or not with RH-OVA or Me49 tachyzoites at 1:1 (5.10⁵ parasites/well) and 1:2 (10⁶ parasites/well) ratios. The culture were set in triplicates and IL12p40 and IL10 concentrations were calculated from the mean of the triplicates. This experiment is representative of one other.

FIG. 9 is a scheme showing two constructions of the scFv anti-DEC205 by anchoring the scFv into the membrane via the GPI of the major surface protein SAG1 (A-B). SS: is a signal sequence of SAG1. GPI: glycosylphosphatidylinositol anchoring domain, linker: GGGAS. ScFv anti-DEC205. Kosak: consensus sequence for the initiation of the translation.

FIG. 10 is a group of image and graphs showing the expression of the RH-DEC205. (A) Western blot membrane image showing the scFv-antiDEC205 expression revealed by anti-HA: at 55 kDa in RH-DC2-SAG1 and at 42 kDa in RH-DC2. (B-C) Cell surface expression of the scFv is measured by ELISA on the whole parasites, using anti-HA antibody and/or anti-T. gondii antibodies.

FIG. 11 is a group of graphs and images showing the fixation of the RH, RH-DC2-SAG1 and RH-DC2 strains on the recombinant DEC205 receptor (CF14) and on dendritic cells. (A-B) Fixation on the recombinant DEC205 receptor measured by ELISA using anti-T. gondii antibodies, analysis of the binding of the RH, RH-DC2-SAG1 and RH- on the recombinant DEC205 receptor (CF14) and on dendritic cells (mutuDC), and by flow cytometry using a monoclonal antibody directed against the T. gondii gp23 surface antigen, fixation of the RH (C), RH-DC2-SAG1 (D) and RH-DC2 (E) on dendritic cells (mutuDC) showed by microscopy using anti-T. gondii antibodies (also indicated by white arrows).

FIG. 12 shows the study scheme of the treatment with RH-DC2-SAG1 or RH ctrl strains (A) and their effects on the tumor volume kinetic (B), the tumor volume (C) and the tumor implantation (D) in mice inoculated or not with the EG7-OVA tumor cell line. (E) Shows an image illustrating the tumor size in mice inoculated with the EG7 control, RH-DC2-SAG1 and RH ctrl strains.

FIG. 13 is a group of diagram, images and graphs and images showing the construction (A), the expression (B-C) and the functionality (D) of the secreted RH-IL15 MICS and RH-IL15 SUB1 strains. (A) Shows the construction of RH-IL15 MICS and RH-IL15 SUB1 strains with the plasmid pUC8 CAT/GFP-IL-15hRec. Signal sequence: is a signal sequence of SUB1 or MICS (SEQ ID NO: 9, 10, 13 or 14). Prodomain sequence: is a prodomain sequence of SUB1 or MICS (SEQ ID NO: 11, 12, 15 or 16). IL15-Rα sushi domain (SEQ ID NO: 3 or 4), linker (SEQ ID NO: 7 or 8). Human IL-15 (SEQ ID NO: 5 or 6). (B) Shows the expression of the IL-15hRec by immunofluorescence assay revealed by anti-IL-15. The specific secretion of human IL-15hRec is measured by ELISA, using human anti-IL-15 (C). (D) Shows graph of the dosage of IFNγ secreted by cells derived from spleen of naive mouse co-cultured or not with different culture supernatants of RH-IL-15hRec clones. The IFNγ secretion was measured by ELISA.

FIG. 14 shows the study scheme of the treatment with NC1-IL-15 strains (A) and its effects the tumor volume (B) in mice inoculated or not with the EG7 tumor cell line.

FIG. 15 shows the study scheme of the glioblastoma treatment with Me49 strain (A) and its effects on the mice survival (B), the tumor volume (C) and the number of metastatic sites (D) in mice inoculated or not with the GL26 tumor cell line.

FIG. 16 shows the study scheme of the lung cancer treatment with RH-OVA strain or RH ctrl strains (A) and their effects on the tumor induction and metastases development (B) in mice inoculated with the B16F10-OVA tumor cell line.

FIG. 17 shows the study scheme of the ovarian cancer treatment with RH-OVA strain (A) and its effects on the volume of ascites (B) and the tumor weight in mice inoculated with the ID8-OVA tumor cell line.

FIG. 18 shows the effect of the treatment with RH-DC2-SAG1, RH-OVA, MIC1-3 KO or RH ctrl strains on the secretion of cytokines: IL-12p40 (A), IL-15 (B), and IL-6 (C) of cells derived from the tumor in mice inoculated with the EG7-OVA tumor cell line.

FIG. 19 shows the effect of the treatment with RH-DC2-SAG1, RH-OVA, MIC1-3 KO or RH ctrl strains on cell populations: PMN cells (A), CD11b+ cells (B), NK cells (C) and Treg cells (D) derived from the tumor in mice inoculated with the EG7-OVA tumor cell line.

FIG. 20 shows the effect of the treatment with RH-DC2-SAG1, RH-OVA, MIC1-3 KO or RH ctrl strains on cell populations: DC (A), PMN (B), monocytes (C), CD4 T cells (D), CD8 T cells (E), NK (F) and Treg (G) derived from the spleen of mice inoculated with the EG7-OVA tumor cell line.

FIG. 21 shows the specific secretion of human IL-15hRec by NC1-IL-15hRec as measured by ELISA using human anti-IL-15 (A); and the secretion of IFNγ by mouse splenocytes infected with NC1-IL15hRec (MOI 1) or NC1 measured by ELISA in the supernatant after 48 h (B).

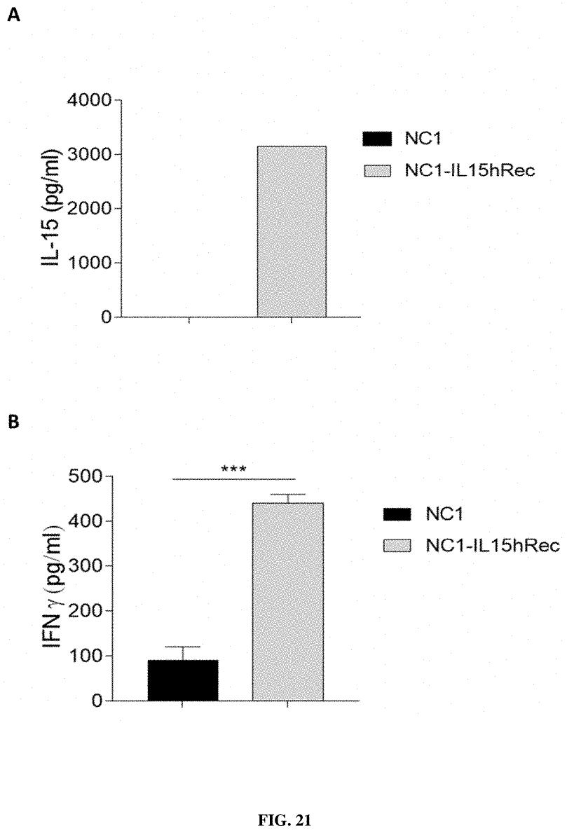

FIG. 22 shows the specific secretion of human IL-15hRec by NC1-IL-15hRec as measured by ELISA using human anti-IL-15 (A); the secretion of IFNγ by human PBMCs infected with NC1-IL15hRec (MOI 1) or NC1 measured by ELISA in the supernatant after 24 h (B); and the percentage of human NK cells measured by analysis of Ki67 expression on human NK cells by flow cytometry (C).

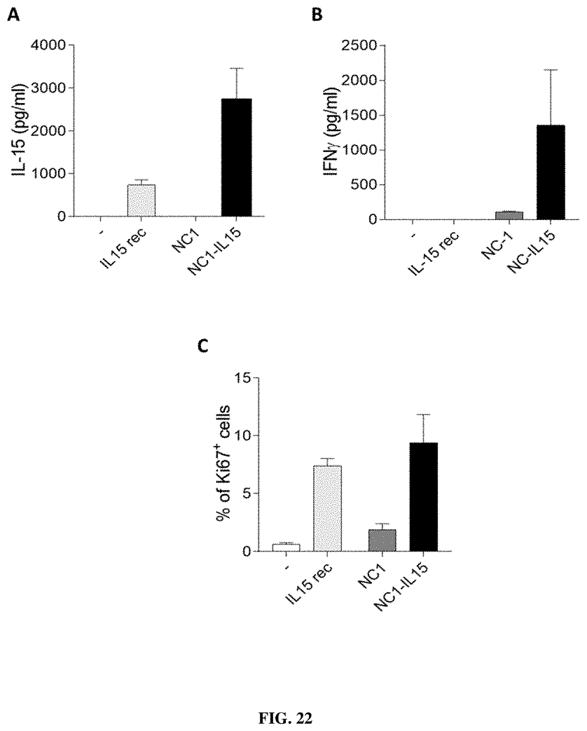

FIG. 23 shows the expression of the RH-anti-hPDL1Rec: cell surface expression of the anti-hPDL1Rec scFv is measured by ELISA on the whole parasites using anti-HA antibody.

### EXAMPLES

The present invention is further illustrated by the following examples.

### Example 1: Specific Expression of Cancer Antigen by the RH Strain and In Vivo Effects

Materials and Methods

Parasites

T. gondii strain RH tachyzoites were produced in human fibroblasts (HFFs) cultured in Dulbecco's minimal medium (DMEM) supplemented with 10% of fetal calf serum, 2 mM glutamine, 50 U/ml of penicillin and 50 μ/ml of streptomycin. They were harvested during lysis of the host cells.

Plasmid Construction of the RH-OVA

The plasmid pUC8.4 CAT/GFP-SAG1/OVA/GPI was used to construct the recombinant RH-OVA.

pUC8.4 CAT/GFP-SAG1/OVA/GPI is a pUC8 plasmid in which the sequence encoding the fusion protein SAG1-OVA including the N terminal signal sequence of SAG1 and the anchor SAG1 signal motif (GPI) is cloned in the expression cassette between PmeI and NotI sites.

pUC8 contains two expression cassettes. One was designed to express a CAT-GFP fusion protein to allow drug selection of stably transfected parasites (cassette CAT-GFP), the second was designed to express proteins of interest. The sequence encoding the protein of interest must include an ATG and a stop codon. The expression of CAT-GFP is driven by the promoter of the T. gondii α-tubulin gene (αTUB5) and the 3′ untranslated region of the T. gondii SAG1 gene. This expression cassette is bordered in the 3′ position and 5′ position by LoxP sites. These LoxP sites were added to suppress the cassette CAT-GFP from the DNA genome of the parasite by the use of a Cre recombinase which recognizes specifically these sites.

The expression of the protein of interest is driven by the promoter of the T. gondii α-tubulin gene (αTUB5) in which a five-repeat element was inserted upstream of the transcriptional start site (leading to promoter αTUB8) for high-level expression of the protein of interest (Soldati et al., 1995). The sequence of the protein of interest is cloned in PmeI/NotI sites.

Obtention of pUC8 and pUC5

Generation of the plasmid pCN1, containing a cassette to express the fusion protein SAG1-GFP driven by the promoter of the T. gondii α-tubulin gene (αTUB5) and the 3′ untranslated region of the T. gondii SAG1 gene (3′UTR SAG1):

The sequence encoding SAG1 (including the signal sequence, without the GPI anchor signal sequence) was amplified by PCR by using plasmid pcDNA3-SAG1 as the template (Mévélec et al., 2005) and the primer sequences GGTTTTGACGTCACCATGTTTCCGAAGGCAGTG (SEQ ID NO: 34) (AaTII, underlined) and TTGCTCACCATCCTAGGTGCAGCCCCGGCAAA (SEQ ID NO: 35) (AvrII, underlined). The sequence encoding GFP was amplified by PCR by using plasmid pmic3-GFP (Striepen et al., 2001) and the primer sequences TTTGCCGGGGCTGCACCTAGGATGGTGAGCAA (SEQ ID NO: 36) (AvrII, underlined) and

(SEQ ID NO: 37)
CGGTGATTAATTAATCGAGCGGGTCCTGGTTCG

(PacI, underlined). SAG1 and GFP PCR products were digested by AaTII/AvrII and AvRII/PacI respectively and ligated into plasmid pT230TUB/Ble (Kim et al., 1993) previously digested with AaTII/PacI. In the resulting plasmid (pCN1), the sequence encoding BLE is replaced by the sequence encoding the secreted fusion protein SAG1-GFP under the control of promoter αTUB5.

Generation of the plasmid pCN5, containing a cassette expressing CAT under the promoter αTUB5, bordered by the Loxp sites in the 3′ position and 5′ position: The sequence of the cassette expressing CAT, driven by the promoter of the T. gondii a-tubulin gene (α-TUB5) and the 3′ untranslated region of the T. gondii SAG1 gene (3′UTR SAG1), was amplified by PCR by using pmic3KO-2 (Cérède et al., 2005) as the template and the primer sequences GCGGCCAAGCTTATAACTTCGTATAATGTATGCTATACGAAGTTATGATATGCAT GTCCGCgttcgtgaaatctctgatcaagcgg (SEQ ID NO: 38) (including HindIII and Loxp sequences, underlined and in italic respectively) and cgacgcacgctgtcactcaacttgctGCTAGAACTAGTGGATCCATAACTTCGTATAGCATAC ATTATACGAAGTTATCCCTCGG (SEQ ID NO: 39) (including SpeI and LoxP sequences, underlined and in italic respectively).

The PCR fragment was digested by HindIII/SpeI and cloned into pCN1 which has been previously digested by the same enzymes HindIII/SpeI. In the resulting plasmid (pCN5), the cassette expressing the fusion protein SAG1-GFP under the control of promoter αTUB5 is replaced by the cassette expressing CAT under the promoter αTUB5, bordered by the Loxp sites in the 3′ position and 5′ position.

Generation of the plasmid pUC18 CAT-GFP containing a cassette to express the fusion protein CAT-GFP driven by the promoter of the T. gondii α-tubulin gene (αTUB5) and the 3′ untranslated region of the T. gondii SAG1 gene (3′UTR SAG1). The cassette is bordered on both sides by Loxp sites:

To clone the sequence encoding GFP in fusion with the sequence encoding CAT, a fragment including LoxP (5′ position), promoter αTUB5 and the sequence encoding CAT without stop codon but with an AvrII site to clone GFP in fusion with CAT, was amplified by PCR by using pCN5 as the template and the primer sequences GTATCGATAAGCTTATAACTC (SEQ ID NO: 40) (HindIII underlined) and CACAACGGTGATTAACCTAGGAGCCCCGCCCTG (SEQ ID NO: 41) (AvrII, underlined). The amplified fragment was digested by HindIII/AvrII. The sequence encoding GFP was obtained from pCN1 by digestion with AvrII/PacI. The plasmid pCN5 was digested by HindIII/PacI to eliminate the HindIII/PacI fragment corresponding to LoxP (5′ position), promoter αTUB5 and the sequence encoding CAT. Finally the three fragments were ligated to obtain a recombinant plasmid containing the cassette expressing CAT-GFP under the promoter αTUB5, bordered by the Loxp sites in the 3′ position and 5′ position. This cassette was further cloned into pUC18 using HindIII and XbaI to obtain pUC18 CAT-GFP.

Generation of pUC8 and pUC5, containing one cassette, bordered by LoxP sites, to express the fusion protein CAT-GFP driven by the promoter of the T. gondii α-tubulin gene (αTUB5) and the 3′ untranslated region of the T. gondii SAG1 gene (3′UTR SAG1) and another one to express a protein of interest driven by a modified promoter of the T. gondii α-tubulin gene (αTUB8) and the 3′ untranslated region of the T. gondii SAG1 gene (3′UTR SAG1). In pUC5 the two cassettes are in the same orientation, in pUC8 they are in opposite side:

The expression cassette with a modified promoter of the T. gondii α-tubulin gene (αTUB8) was obtained by addition of five repeat sequences (Soldati et al., 1995), 70 bases upstream the major transcription start site in the T. gondii α-tubulin gene (αTUB5). Plasmid pT230TUB/Ble was used to obtain the promoter αTUB8 and the 3′UTR SAG1 sequences. Two enzymes restriction sites PmeI and NotI were included between the αTUB8 and 3′UTR SAG1 sequences to allow insertion of the sequence encoding the protein of interest. The XbaI restriction sites located at both end of the expression cassette were used to clone this expression cassette in pUC18 CAT-GFP in both orientations. The resulting plasmids were pUC5 with the two cassettes in the same orientation and pUC8 with the two cassettes in opposite side.

Generation of Plasmid pUC8.4 CAT/GFP-SAG1/OVA/GPI

pUC8.4 CAT/GFP-SAG1/OVA/GPI is a pUC8 plasmid in which the sequence encoding the fusion protein SAG1-OVA including the N terminal signal sequence of SAG1 and the anchor SAG1 signal motif (GPI) is cloned in the expression cassette between PmeI and NotI sites. The fusion protein is expressed under the control of αTUB8. The anchor motif was added to the C terminus of OVA to achieve the retention of the fusion protein SAG1-OVA in the plasma membrane of the parasite.

The OVA fragment encoding amino acids 140 to 386 of chicken ovalbumin was amplified by PCR from an OVA containing plasmid template (Tagliani et al., 2008) using the primer sequences CAAACACCTAGGGATCAAGCCAGAGAGC (SEQ ID NO: 42) (AvrII, underlined) and GTTCCCTAGGGGAAACACATCTGCC (SEQ ID NO: 43) (AvrII, underlined). The amplified fragment was digested with AvrII and cloned into pCN1 previously linearized with AvrII and dephosphorylated. In the resulting plasmid (pCN1-OVA), the sequence encoding OVA is inserted in frame between the sequence encoding SAG1 and the sequence encoding GFP. pCN1-OVA expresses the fusion protein SAG1-OVA-GFP driven by the promoter of the T. gondii α-tubulin gene (αTUB5) and the 3′ untranslated region of the T. gondii SAG1 gene (3′UTR SAG1).

The sequence encoding the fusion protein SAG1-OVA (including the Kozak sequence, the start ATG codon and the N terminal signal sequence of SAG1, without the stop codon) was amplified by PCR using pCN1-OVA as the template and the primer sequences GGTGCTCACCGGTTTAAACGTCGAAAATGTTTCCG (SEQ ID NO: 44) (PmeI, underlined) and CTCACCATTCTAGAGGAAACACATCTGC (SEQ ID NO: 45) (XbaI, underlined). The sequence encoding the anchor signal (GPI), including the stop codon, was amplified by PCR using pcDNA3-SAG1 (Mevelec et al., 2005) and the primer sequences CTCATCTCTAGAGAGGATCTGGCTGCGGG (SEQ ID NO: 46) (XbaI, underlined) and

(SEQ ID NO: 47)
ACCATGGAAGCGGCCGCTTACGCGACA

(NotI, underlined). SAG1-OVA and GPI PCR products were digested with XbaI and ligated. The ligated product was amplified with GGTGCTCACCGGTTTAAACGTCGAAAATGTTTCCG (SEQ ID NO: 44) (PmeI, underlined) and

(SEQ ID NO: 47)
ACCATGGAAGCGGCCGCTTACGCGACA

(NotI, underlined) and cloned into plasmid pGEMT (Promega). The resulting plasmid pGEMT-SAG1-OVA-GPI was then digested with PmeI/NotI to get the fragment encoding SAG1-OVA-GPI. The SAG1-OVA-GPI fragment was cloned in pUC8 previously opened by PmeI/NotI.

The resulting plasmid pUC8.4 CAT/GFP-SAG1/OVA/GPI expresses the membrane anchored fusion protein SAG1-OVA under the control of promoter αTUB8 and the fusion protein CAT-GFP under the control of promoter αTUB5 to allow drug selection of stably transfected parasites.

Recombinant Toxoplasma gondii Strain

Transfections were performed with 10⁷ parasites in a volume of 800 μl of cytomix (Van den Hoff et al., 1992) containing 3 mM ATP and 3 mM glutathione and 20 ρg of purified plasmid DNA (the plasmids were purified using the Qiagen Kit®), linearized with PciI. Electroporations were performed in disposable cuvettes (4 mm gap) with an electroporator Biorad (electroporation settings: 2000 V, 50 ohms, 25 mF). After electroporation, the parasites are kept in the hood, for 15 min at room temperature and then transferred to a fresh culture of fibroblast monolayers (25 cm² flask). After 24 hours the parasite are subjected to 20 mM chloramphenicol selection. After 10 to 15 days of selection, the parasites are cloned by limiting dilution, in the wells of a 96-well plate, of HFF cells, in the presence of selection agent, and the clones selected are amplified.

Immunofluorescence Assays

T. gondii tachyzoites filtered through a 5 μM Nucleopore filter, pelleted by centrifugation, washed 2 times with PBS, were air dried on standard IFA slides (10⁵ parasites/well) and were stored at −20° C. Immunofluorescence assays were performed at 37° C. after 2 min fixation in cold acetone (−20° C.). Fixed tachyzoites were washed in PBS and incubated 60 mM with polyclonal anti-chicken egg antibodies produced in rabbit (Sigma, 1:50). Slides were then washed with PBS and incubated 60 min with a 1:1000 dilution in PBS of Alexa Fluor 568-conjugated goat anti-rabbit IgG antibodies. After washes in PBS, the visualization was carried out using a Leica microscope.

Mice

Twenty-four week-old female C57BL/6 mice were purchased from CER Janvier (Le Genest Saint Isle, France) and maintained under pathogen-free conditions in the animal house of the University of Tours. Experiments were carried out in accordance with the guideline for animal experimentation (EU Directive 2010/63/EU) and the protocol was approved by the local ethics committee (CEEA VdL).

Toxoplasma gondii Strains (RH-OVA)

Strain RH-OVA tachyzoites were produced in HFFs cultured in DMEM (Pan Biotech GmbH) supplemented with 10% of heat-inactivated FCS (Dutscher), 2 mM glutamine (Pan Biotech GmbH), 50 U/ml of penicillin and 50 μ/ml of streptomycin (Pan Biotech GmbH) at 37° C. in 5% CO2 atmosphere. They were harvested during lysis of the host cells by centrifugation at 600 g for 10 min.

Tumor Cells and Tumor Cell Inoculation (EG7)

EG7 cells (EL4-OVA thymoma cells transfected with chicken albumin cDNA) are cultured in Roswell Park Memorial Institute medium (RPMI, Pan Biotech GmbH) with 5×10⁵ M of 2-mercaptoethanol, 50 UI/mL of penicillin and 50 mg/mL of streptomycin. 5×10⁵ live EG7 cells are inoculated subcutaneously in the right flank of mice. Tumor diameters are measured 3 times weekly, and mice are euthanized when tumor diameters reached 25,000 mm³.

T. gondii Administration

Mice are injected subcutaneously in the right flank at day 4 and again at day 7 with 5×10² freshly isolated tachyzoites of RH ctrl and RH-OVA strain of T. gondii.

Spleens and Lymph Nodes Cytokine Measurements

Single cell suspensions were obtained from spleens and lymph nodes first pressed and then filtered through a nylon mesh. Hypotonic shock was used to remove erythrocytes. The cells were then resuspended in RPMI medium supplemented with 5% FBS, 25 mM HEPES, 2 mM glutamine, 1 mM sodium pyruvate, 50 μM 2 β-mercaptoethanol and 1 mM penicillin-streptomycin. Cells were dispensed, in triplicate, into 96-well culture plates (5 10⁵ cells/well) and cultured for 72 hours with or without 10 μg/mL OVA. Concanavalin A (5 μg/mL) was used as positive control of proliferation. Cytokine productions in supernatants from restimulated splenocytes and lymph nodes cells were evaluated using cytometric bead array for IFN-γ, TNF-α, Il-2, Il-4, Il-5, Il-10, Il-17A, IL-23 and GM-CSF (MACSPlex Cytokine 10 kit mouse, Miltenyi Biotec GmbH, Germany), commercial ELISA kits for TGF-β and IL-15 (Invitrogen, Thermo Fisher Scientific, U.S.A.).

Tumor Cytokine Measurements

Tumors were cut into small pieces of 2-4 mm and dissociated with Tumor Dissociation Kit and gentleMACS Dissociator (Miltenyi Biotech GmbH, Germany) Following dissociation, cells were pelleted and supernatant were collected for cytokine analysis. Cytokines were quantified in the supernatant using cytometric bead array for IFN-γ, TNF-α, Il-2, Il-4, Il-5, Il-10, Il-17A, IL-23 and GM-CSF (MACSPlex Cytokine 10 kit mouse, Miltenyi Biotec GmbH, Germany), commercial ELISA kits for TGF-β and IL-15 (Invitrogen, Thermo Fisher Scientific, U.S.A.).

Cell Populations Phenotyping

Spleen and tumor cell phenotypes were analyzed by flow cytometry. Spleen cells were obtained as described for cytokine measurements. Spleen cells were then pelleted and resuspended in PBS, 2 mM EDTA, 1% FBS for staining and cytometry analysis. Red blood cells from tumor cells obtained as described for cytokine measurements were lysed using red blood cell lysis buffer and cells were resuspended in PBS, 2 mM EDTA, 1% FBS for staining and cytometry analysis. Cells were stained with monoclonal antibodies purchased from Miltenyi Biotec GmbH. The following antibodies were used: REA clones of anti-mouse CD3-APC-Vio770 (REA606), CD4-Vioblue (REA 604), CD8a-PE-Vio770 (REA 601), Foxp3-Vio515 (REA 788), NKp46-APC (REA 815), CD11c-PE (REA 754), CD11b-APC-Vio770 (REA 592), Ly6C-Vioblue (REA 796), Ly6G-PE-Vio770 (REA 526). Foxp3 staining was performed using Foxp3 Staining Buffer Set (Miltenyi Biotech GmbH, Germany) FACS analysis was performed using a Miltenyi 8-color MACSQuant, and data were analyzed using Flowlogic (Milteni Biotech GmbH, Germany).

In Vitro Generation of Tolerogenic Dendritic Cells (DCs)

Bone marrow dendritic cells were obtained essentially as described previously (Madaan et al., Journal of Biological Methods, Vol. 1, 2014). Bone marrow cells collected from femur and tibias of mice were plated at 4×10⁶ cells/mL in low petri dishes and cultured at 37° C., 5% CO₂ in 10 mL complete medium [RPMI-1640 medium containing 10% heat-inactivated fetal bovine serum (FBS), 10 mM HEPES, 1 mM pyruvate, 2 mM 1-glutamine, 100 U/mL penicillin, streptomycin (All from PAN BioTech), 1% non-essential amino acids (GIBCO), and 50 μM 2β-mercaptoethanol], and 5 ng/mL of GMCSF from J558L cells supernatant to generate mouse bone marrow dendrtiic cells. Medium was replaced every 2 days. To promote tolerogenic dendritic cells, 1,25dihydroxyvitamin D3 (Sigma) was added on days 0, 2, 4, 6 at 10⁻⁸M as previously described (Ferreira et al., Journal of Immunology, 192(9):4210-20, 2014). Cells were recovered at day 7 and plated at 5.10⁵ cells/ml in 24-wells culture plates in a final volume of 1 ml of RPMI containing 2% FBS, 10 mM HEPES, 1 mM pyruvate, 2 mM 1-glutamine, 100 U/mL penicillin, streptomycin and 50 μM 2β-mercaptoethanol]. Parasites were added at multiplicity of infection (MOI) 1 (5.10⁵) or 2 (10⁶) and incubated for an additional 18 hours period. Supernatants were collected and 11-12 and IL-10 cytokine productions were evaluated using commercial ELISA kits according to the manufacturer's instructions (Invitrogen, Thermo Fisher).

Results

SAG1-OVA Plasmid Construct

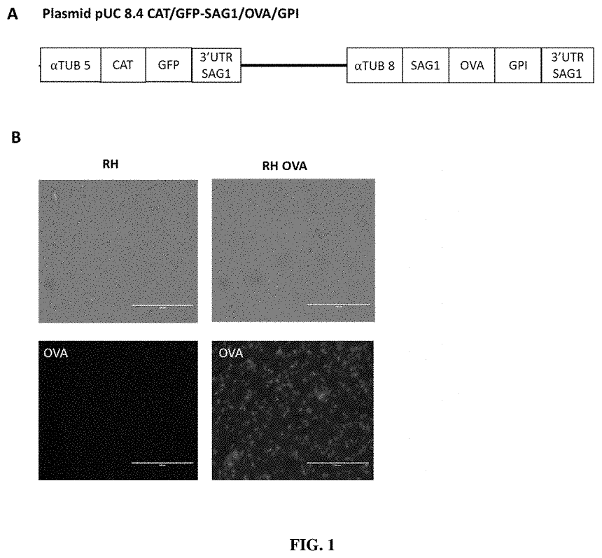

The plasmid pUC8.4 was constructed as described in FIG. 1A and below:

Briefly, plasmid pUC8.4 is a pUC18 plasmid in which two cassettes have been cloned. One cassette encodes the fusion protein CAT-GFP to select stably transfected tachyzoites with chloramphenicol. The sequence encoding the fusion protein CAT-GFP is flanked by the promoter T. gondii tubulin gene (a TUB 5) and the 3′ untranslated region of the SAG1 gene (3′UTR SAG1). The second cassette expresses a fusion protein consisting of the complete T. gondii SAG1 fused to the amino acids 140-386 of chicken ovalbumin enchored in the plasma membrane. To achieve the retention of SAG1-OVA in the plasma membrane, the SAG1 GPI motif has been added to the C terminus of OVA. The sequence encoding the fusion protein SAG1-OVA and the GPI motif is flanked by a modified promoter T. gondii tubulin gene (αTUB8) and the 3′ untranslated region of the SAG1 gene (3′UTR SAG1). Then, the T. gondii strain RH tachyzoites were transfected with plasmid pUC8.4.

Expression of the Fusion Protein SAG1-OVA

An immunofluorescence assay was preformed to confirm that the transfected RH strains express SAG1-OVA on their cell surface. Briefly, air dried, free T. gondii tachyzoites non-transfected (RH) or transfected (RH-OVA) with the plasmid PUC8.4 were fixed on slides with acetone, washed with PBS and stained with rabbit anti-chicken ovalbumin followed by ALEXA 568-conjugated goat anti-rabbit antibodies. Slides were examined under a Leica microscope.

As shown in FIG. 1B, T. gondii strain RH tachyzoites transfected with plasmid pUC8.4 express at their surface the OVA antigen.

RH-OVA Treatment Suppresses and/or Regresses an Established Solid Tumor Development

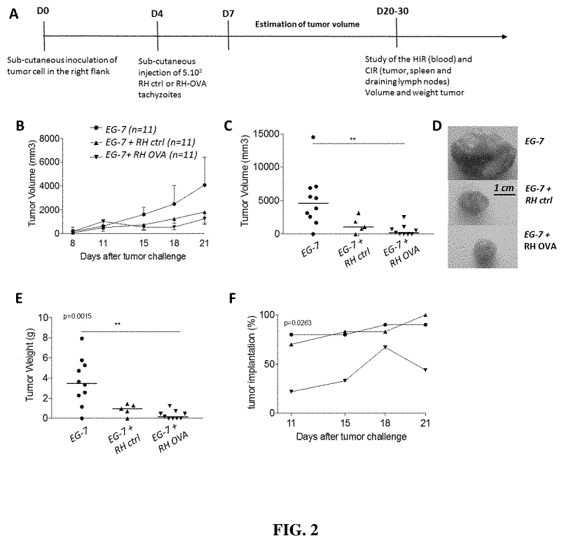

As shown in FIG. 2, treatment with 500 tachyzoites of the RH-OVA strain by peritumoraly route decreased tumor development (i.e., volume and weight) in mice. Indeed, tumor volume and tumor weight in mice treated with RH or RH-OVA strains was significantly lower than those in non-treated mice (FIG. 2B-C-D-E). Even if differences of protection between RH and RH-OVA treatments are not statistically significant, we can observe a slight tendency to a better protection for RH-OVA.

Moreover, a significant reduction of tumor implantation was observed in mice treated with RH-OVA (FIG. 2F).

All these results suggest that a sub-cutaneous injection at peritumoral level of RH-OVA tachyzoites exhibited good efficacy against tumor development and seems to confirm the benefit to express specific tumor antigen by our construct.

RH-OVA Induces a Protective Immune Response Against Tumor Development

The administration of RH-OVA tachyzoites at the site of the tumor, D4 and D7 post EG-7 thymoma injection induce protective immune responses at splenic and tumor levels.

a) Systemic Compartment (i.e., Spleen)

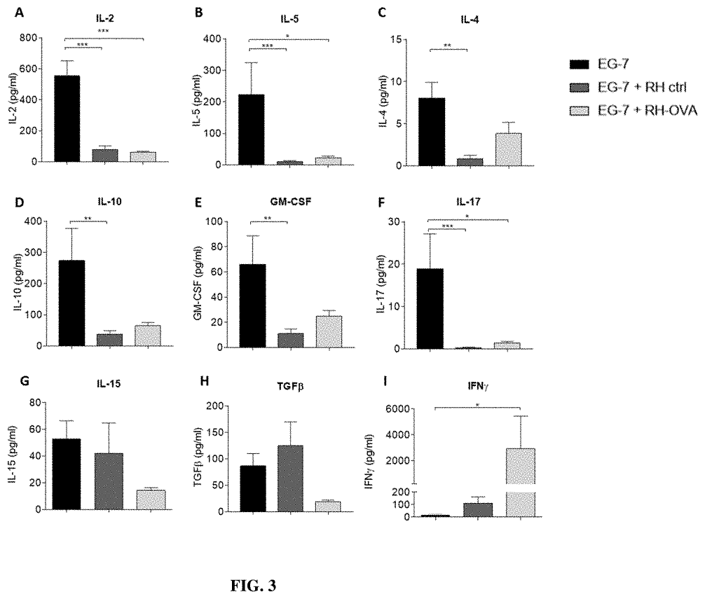

As described in FIGS. 3A-G, RH and RH-OVA treatments induce significant decrease of secretion of different cytokines (IL-2, IL-5, IL-4, IL-10, GM-CSF, IL-17A, IL-15 and TGF-β) in comparison to cytokine secretion by spleen from EG-7 tumor-mice. This reduction is more important for the RH-OVA group.

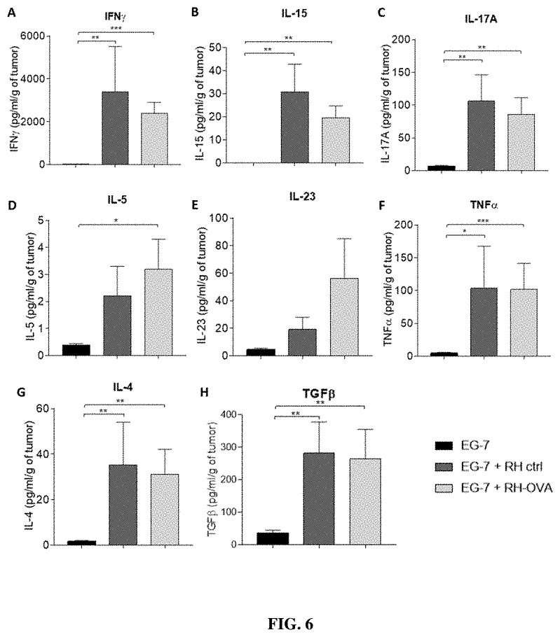

Concerning IFN-γ, we observe an increase of secretion in spleen (FIG. 3I), brachial lymph node and inguinal lymph node (FIG. 5) of the RH and RH-OVA mice. These results confirm that expression of specific tumor antigen is pertinent to induce IFN-γ production, cytokine of good prognostic in tumor regression.

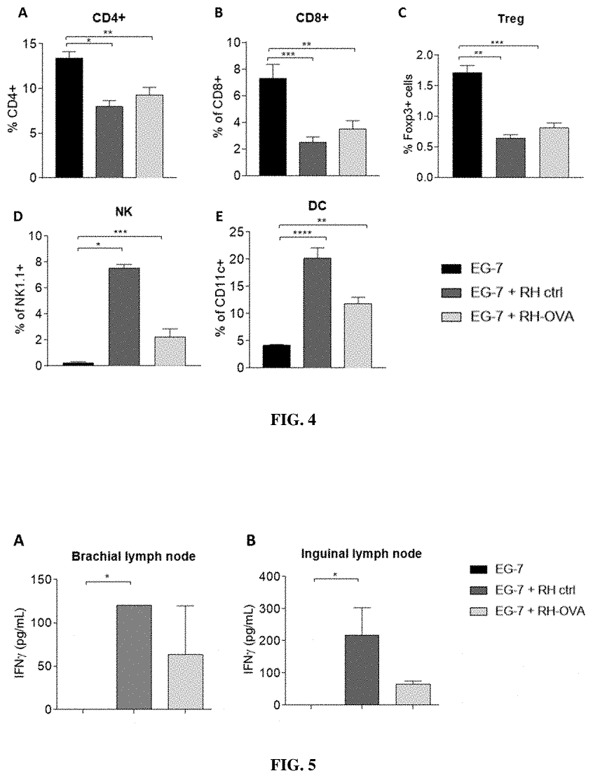

Concerning spleen myeloid cell sub-populations, we observe a significant increase of CD11c+/CMH II+ cells (dendritic cells) (FIG. 4E), of Ly6C+Ly6G % (neutrophils) and CD11b+(monocytes) (data not shown).

For spleen lymphoid cell sub-populations, as shown in FIG. 4C-D, we observe an increase of NK cell population (NKp46+ cells) and a decrease of Treg cells (Foxp3+).

b) Tumor Compartment

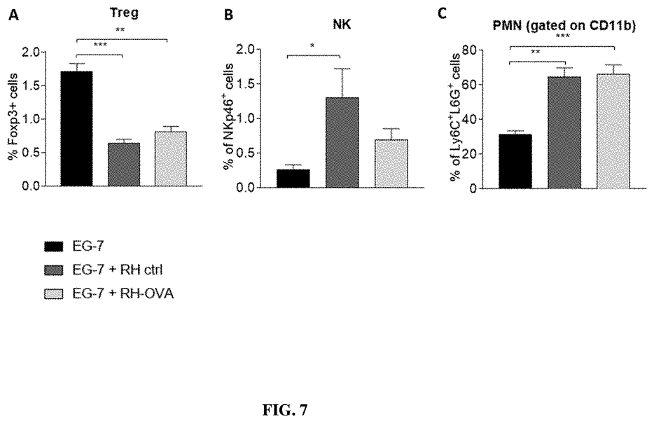

As described in FIG. 6, RH and RH-OVA treatments induce significant increase of secretion of different cytokines (IFN-γ, IL-15, IL-17A, IL-5, IL-23, TNF-α, IL-4, and TGFβ) in comparison to cytokine secretion by tumor from EG-7-mice. We can observe that, for some cytokines, this augmentation is more important for the RH-OVA group: IFN-γ, IL-5, IL-23 and TNF-α.

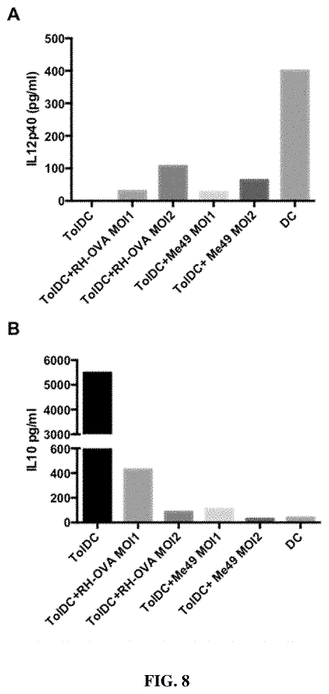

Concerning tumor myeloid cell sub-populations, we observe a significant increase of Ly6C+Ly6G % (neutrophils) (FIG. 7C) and CD11b+ (monocytes) (data not shown) for the two treated groups.

For tumor lymphoid cell sub-populations, as shown in FIG. 7A-B, we observe an increase of NK cell population (NKp46+ cells) and a decrease of Treg cells (Foxp3+) for the two treated groups.

RH-OVA Reactivates Tumor-Altered Dendritic Cells

The active form of the vitamin D3, 1,25-Dihydroxyvitamin D3 (1.25-(OH)₂D₃) can affect the function and development of monocytes and dendritic cells (DCs). Thus, DCs differentiated in the presence of 1,25-vitD3 share several features with tolerogenic DCs like low surface expression of MHC class II and costimulatory molecules (e.g., CD40, CD80, and CD86), decreased production of IL-12, and enhanced secretion of IL-10.

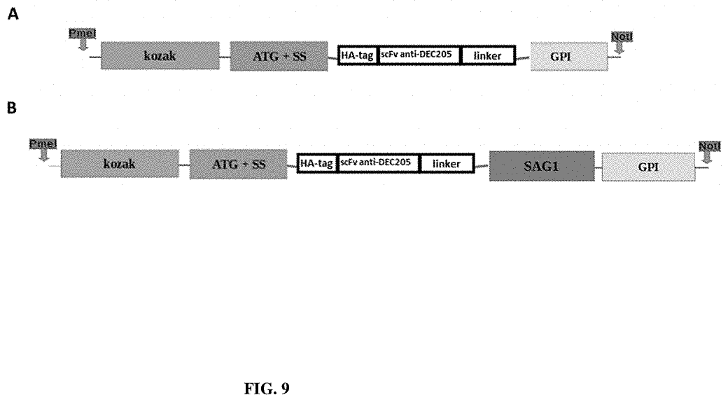

Indeed, as shown in FIG. 8, DCs generated in the presence of 1.25(OH)₂D3, called TolDCs, showed altered cytokine expression patterns with decreased IL12p40 production (FIG. 8A) and increased IL10 production compared (FIG. 8B) to control DC.

Interestingly, parasites adjunction on the TolDCs partially restored the IL12p40 secretion and decreased the IL10 levels. No significant difference was observed in cytokines secretion between type 1 (RH-OVA) and type 2 (Me49) parasites. No TGFβ secretion was detected and IL6 levels were not significantly different between the control DCs or TolDCs (data not shown).

These results suggest that parasites are able to reverse the tolerogenicity induced by 1.25(OH)₂D3.

### Example 2: Specific Targeting of Dendritic Cells by the Strain and In Vivo Effects

Materials and Methods

Parasites

T. gondii strain RH tachyzoites were produced in human fibroblasts (HFFs) cultured in Dulbecco's minimal medium (DMEM) supplemented with 10% of fetal calf serum, 2 mM glutamine, 50 U/ml of penicillin and 50 μ/ml of streptomycin. They were harvested during lysis of the host cells

Plasmid Construction of the RH-DC2 and RH-DC2-SAG1

The plasmids pUC8SSDC2GPI and pUC8SSDC2SAG1GPI were used to construct the recombinant RH-DC2 and RH-DC2-SAG1 respectively.

pUC8SSDC2GPI is a pUC8 plasmid in which the sequence encoding the membrane anchored ScFv DEC 205 (DC2) including a HA tag, is cloned in the expression cassette between PmeI and NotI sites.

pUC8SSDC2SAG1GPI is a pUC8 plasmid in which the sequence encoding the membrane anchored SAG1 ScFv DEC 205 (DC2 SAG1) including a HA tag is cloned in the expression cassette between PmeI and NotI sites.

pUC8 contains two expression cassettes. One was designed to express a CAT-GFP fusion protein to allow drug selection of stably transfected parasites (cassette CAT-GFP), the second was designed to express proteins of interest. The sequence encoding the protein of interest must include an ATG and a stop codon. The expression of CAT-GFP is driven by the promoter of the T. gondii α-tubulin gene (αTUB5) and the 3′ untranslated region of the T. gondii SAG1 gene. This expression cassette is bordered in the 3′ position and 5′ position by LoxP sites. These LoxP sites were added to suppress the cassette CAT-GFP from the DNA genome of the parasite by the use of a Cre recombinase which recognizes specifically these sites.

The expression of the protein of interest is driven by the promoter of the T. gondii α-tubulin gene (αTUB5) in which a five-repeat element was inserted upstream of the transcriptional start site (leading to promoter αTUB8) for high-level expression of the protein of interest (Soldati et al., 1995). The sequence of the protein of interest is cloned in PmeI/NotI sites.

Obtention of pUC8 and pUC5

Generation of the plasmid pCN1, containing a cassette to express the fusion protein SAG1-GFP driven by the promoter of the T. gondii α-tubulin gene (αTUB5) and the 3′ untranslated region of the T. gondii SAG1 gene (3′UTR SAG1): The sequence encoding SAG1 (including the signal sequence, without the GPI anchor signal sequence) was amplified by PCR by using plasmid pcDNA3-SAG1 as the template (Mevelec et al., 2005) and the primer sequences GGTTTTGACGTCACCATGTTTCCGAAGGCAGTG (SEQ ID NO: 34) (AaTII, underlined) and TTGCTCACCATCCTAGGTGCAGCCCCGGCAAA (SEQ ID NO: 35) (AvrII, underlined). The sequence encoding GFP was amplified by PCR by using plasmid pmic3-GFP (Striepen et al., 2001) and the primer sequences TTTGCCGGGGCTGCACCTAGGATGGTGAGCAA (SEQ ID NO: 36) (AvrII, underlined) and

(SEQ ID NO: 37)
CGGTGATTAATTAATCGAGCGGGTCCTGGTTCG

(PacI, underlined). SAG1 and GFP PCR products were digested by AaTII/AvrII and AvRII/PacI respectively and ligated into plasmid pT230TUB/Ble (Kim et al., 1993) previously digested with AaTII/PacI. In the resulting plasmid (pCN1), the sequence encoding BLE is replaced by the sequence encoding the secreted fusion protein SAG1-GFP under the control of promoter αTUB5.

Generation of the plasmid pCN5, containing a cassette expressing CAT under the promoter αTUB5, bordered by the Loxp sites in the 3′ position and 5′ position: The sequence of the cassette expressing CAT, driven by the promoter of the T. gondii a-tubulin gene (α-TUB5) and the 3′ untranslated region of the T. gondii SAG1 gene (3′UTR SAG1), was amplified by PCR by using pmic3KO-2 (Cérède et al., 2005) as the template and the primer sequences GCGGCCAAGCTTATAACTTCGTATAATGTATGCTATACGAAGTTATGATATGCAT GTCCGCgttcgtgaaatctctgatcaagcgg (SEQ ID NO: 38) (including HindIII and Loxp sequences, underlined and in italic respectively) and cgacgcacgctgtcactcaacttgctGCTAGAACTAGTGGATCCATAACTTCGTATAGCATAC ATTATACGAAGTTATCCCTCGG (SEQ ID NO: 39) (including SpeI and LoxP sequences, underlined and in italic respectively).

The PCR fragment was digested by HindIII/SpeI and cloned into pCN1 which has been previously digested by the same enzymes HindIII/SpeI. In the resulting plasmid (pCN5), the cassette expressing the fusion protein SAG1-GFP under the control of promoter αTUB5 is replaced by the cassette expressing CAT under the promoter αTUB5, bordered by the Loxp sites in the 3′ position and 5′ position.

Generation of the plasmid pUC18 CAT-GFP containing a cassette to express the fusion protein CAT-GFP driven by the promoter of the T. gondii α-tubulin gene (αTUB5) and the 3′ untranslated region of the T. gondii SAG1 gene (3′UTR SAG1). The cassette is bordered on both sides by Loxp sites:

To clone the sequence encoding GFP in fusion with the sequence encoding CAT, a fragment including LoxP (5′ position), promoter αTUB5 and the sequence encoding CAT without stop codon but with an AvrII site to clone GFP in fusion with CAT, was amplified by PCR by using pCN5 as the template and the primer sequences GTATCGATAAGCTTATAACTC (SEQ ID NO: 40) (HindIII underlined) and CACAACGGTGATTAACCTAGGAGCCCCGCCCTG (SEQ ID NO: 41) (AvrII, underlined). The amplified fragment was digested by HindIII/AvrII. The sequence encoding GFP was obtained from pCN1 by digestion with AvrII/PacI. The plasmid pCN5 was digested by HindIII/PacI to eliminate the HindIII/PacI fragment corresponding to LoxP (5′ position), promoter αTUB5 and the sequence encoding CAT. Finally the three fragments were ligated to obtain a recombinant plasmid containing the cassette expressing CAT-GFP under the promoter αTUB5, bordered by the Loxp sites in the 3′ position and 5′ position. This cassette was further cloned into pUC18 using HindIII and XbaI to obtain pUC18 CAT-GFP.

Generation of pUC8 and pUC5, containing one cassette, bordered by LoxP sites, to express the fusion protein CAT-GFP driven by the promoter of the T. gondii α-tubulin gene (αTUB5) and the 3′ untranslated region of the T. gondii SAG1 gene (3′UTR SAG1) and another one to express a protein of interest driven by a modified promoter of the T. gondii α-tubulin gene (αTUB8) and the 3′ untranslated region of the T. gondii SAG1 gene (3′UTR SAG1). In pUC5 the two cassettes are in the same orientation, in pUC8 they are in opposite side:

The expression cassette with a modified promoter of the T. gondii α-tubulin gene (αTUB8) was obtained by addition of five repeat sequences (Soldati et al., 1995), 70 bases upstream the major transcription start site in the T. gondii α-tubulin gene (αTUB5). Plasmid pT230TUB/Ble was used to obtain the promoter αTUB8 and the 3′UTR SAG1 sequences. Two enzymes restriction sites PmeI and NotI were included between the αTUB8 and 3′UTR SAG1 sequences to allow insertion of the sequence encoding the protein of interest. The XbaI restriction sites located at both end of the expression cassette were used to clone this expression cassette in pUC18 CAT-GFP in both orientations. The resulting plasmids were pUC5 with the two cassettes in the same orientation and pUC8 with the two cassettes in opposite side.

Generation of the Plasmid pUC8SSDC2GPI

pUC8SSDC2GPI expresses the membrane anchored ScFv DEC 205 (DC2) protein, including a HA tag, under the control of promoter αTUB8 and CAT-GFP under the control of promoter αTUB5. The sequence encoding the membrane anchored ScFv DEC 205 (DC2) including a HA tag, is cloned in the PmeI and NotI sites of pUC18.

The sequence encoding the N terminal signal sequence of SAG1 (SEQ ID NO: 21), including the Kozak sequence and the start ATG was amplified by PCR using pGEMT-SAG1-OVA-GPI as the template and the primer sequences G GTG CTCA CCG GTT TAA ACG TCG AAA ATG TTT CCG (SEQ ID NO: 44) (PmeI, underlined) and GGC AAC ACT AGT GGG ATC CGA TGC (SEQ ID NO: 48) (SpeI, underlined). The sequence encoding the ScFv anti-DEC 205 including an N-terminal HA (hemagglutinin) tag (SEQ ID NO: 33), and a linker at the C terminal end (SEQ ID NO: 28) was amplified by PCR using pMT DA2.2 (from our laboratory: this plasmid contains the VH and VL regions of monoclonal antibody NLDC 145, obtained by RT-PCR from total RNA of hybridome NLDC 145 ATCC 1996) as the template with the primer sequences CTCGGGACTAGTTACCCATACGATGTTCCAGATTACGCTCAGGTGCAGCTGC AGGAGAG (SEQ ID NO: 49) (SpeI and HA sequences, underlined and in italic respectively) and ATTGGCCACTCTAGAGCTAGCGCCTCCGCCCTTCAGC (SEQ ID NO: 50) (XbaI and liker, underlined and in italic respectively). SAG1 N terminal signal sequence and ScFv PCR products were digested with SpeI and ligated. The ligated product was amplified with

(SEQ ID NO: 44)
G GTG CTCA CCG GTT TAA ACG TCG AAA ATG TTT CCG

(PmeI, underlined) and ATTGGCCACTCTAGAGCTAGCGCCTCCGCCCTTCAGC (XbaI and linker, underlined and in italic respectively) and cloned into plasmid pGEMT (Promega). The resulting plasmid pGEMT-SS SAG1-HA-ScFv-linker was then digested with PmeI/XbaI to get the fragment encoding SS SAG1-HA-ScFv-linker. The sequence encoding the SAG1 anchor signal (GPI) (SEQ ID NO: 18), including the stop codon, was amplified by PCR using pcDNA3-SAG1 (Mévélec et al., 2005) and the primer sequences CTCATCTCTAGAGAGGATCTGGCTGCGGG (SEQ ID NO: 46) (XbaI, underlined) and

(SEQ ID NO: 47)
ACCATGGAAGCGGCCGCTTACGCGACA

(NotI, underlined). The fragment encoding SS SAG1-HA-ScFv-linker obtained from pGEMT-SS SAG1-HA-ScFv-linker and the GPI PCR product were digested with XbaI, ligated, then amplified by PCR with the sequence primers G GTG CTCA CCG GTT TAA ACG TCG AAA ATG TTT CCG (SEQ ID NO: 44) (PmeI, underlined) and ACCATGGAAGCGGCCGCTTACGCGACA (SEQ ID NO: 47) (NotI, underlined). The amplified fragment was cloned into pGEMT (Promega) to obtain pGEMT-SS SAG1-HA-ScFv-linker-GPI. Finally, pGEMT-SS SAG1-HA-ScFv-linker-GPI was digested with PmeI/NotI to get the fragment encoding SS SAG1-HA-ScFv-linker-GPI (DC2) and the DC2 fragment was inserted into pUC8, previously opened by PmeI/NotI. The resulting plasmid pUC8SSDC2GPI expresses the membrane anchored ScFv DEC 205 (DC2) including a HA tag under the control of promoter αTUB8 and CAT-GFP under the control of promoter αTUB5.

Generation of the Plasmid pUC8SSDC2SAG1GPI

pUC8SSDC2SAG1GPI expresses the membrane anchored SAG1 ScFv DEC 205 (DC2) protein, including a HA tag, under the control of promoter αTUB8 and CAT-GFP under the control of promoter αTUB5. The sequence encoding the membrane anchored SAG1 ScFv DEC 205 (DC2) including a HA tag, is cloned in the PmeI and NotI sites of pUC18. To obtain this plasmid, the sequence encoding SAG1 without the N terminal signal sequence and without the stop codon was cloned in frame at the C terminus of the linker (located at the C terminus of the sequence encoding ScFv DEC 205) using the XbaI site.

The sequence encoding SAG1 without the N terminal signal sequence and without a stop codon was amplified by PCR using pGEMT-SAG1-OVA-GPI as the template and the primer sequences ATGGCATCGTCTAGACCTCTTGTTGCCAAT (SEQ ID NO: 51) (XbaI underlined) and CCTAGGTGCTCTAGAGGCAAACTCCAG (SEQ ID NO: 52) (XbaI underlined). The PCR product was cloned into pGEMT (Promega). The resulting plasmid pGEMT-SAG1t was digested with XbaI to get the fragment encoding the truncated SAG1 protein (SEQ ID NO: 23). This fragment was then cloned in pGEMT SS DC2 GPI previously opened with XbaI. pGEMT SS DC2 SAG1 GPI with SAG1 in the proper orientation was selected by restriction mapping. Finally, pGEMT SS DC2 SAG1 GPI with SAG1 in the proper orientation was digested by PmeI/NotI to get the fragment encoding SS SAG1-HA-ScFv-SAG1-linker-GPI (DC2 SAG1) and this fragment was inserted into pUC8, previously opened by PmeI/NotI. The resulting plasmid pUC8SSDC2SAG1GPI expresses the membrane anchored ScFv DEC 205 (DC2 SAG1) including a HA tag under the control of promoter αTUB8 and CAT-GFP under the control of promoter αTUB5.

Western Blot

Electrophoresis (SDS-PAGE) was performed according to Laemmli. The parasites were heated before electrophoresis in sample buffer with SDS and 0.1 M DTT (reduced conditions). The lysates (2 10⁵ or 5 10⁵ tachyzoites/well) were separated on 10% acrylamide gels by sodium dodecyl sulfate polyacrylamide gel electrophoresis. Proteins were transferred to nitrocellulose and were probed as described previously (Chardes et al., Infection and Immunity, 1990) with rabbit anti-HA polyclonal antibodies (1:200, ThermoFisher Scientific) followed by a mouse monoclonal anti-rabbit IgG (γ-chain specific) alkaline phosphatase conjugate (1:4000, Sigma). Alkaline phosphatase activity was detected using the 5-bromo-4-chloro-3-indolyl phosphate/nitroblue tetrazolium (BCIP/NBT) liquid substrate system (Sigma). Molecular mass standards ProSieveQuadColor (Lonza) were used.

ELISA on T. gondii Parasites

ELISA was performed on whole tachyzoites, essentially as described previously (Chardes et al., Infection and immunity, 1990). Flat bottomed wells (96-well plate, NUNC) were coated with 2 10⁵ parasites/well in PBS. After centrifugation at 200×g and 4° C. for 5 min, 25 μL of 0.5% of glutaraldehyde in cold PBS was added to each well and left for 8 min at room temperature. The plates were washed twice in PBS and saturated with PBS-4% BSA for 1 h at 37° C. Rabbit anti-HA polyclonal antibodies (1:400, ThermoFisher Scientific) or serum from infected T. gondii rabbit (1:2000) in PBS-1% BSA were incubated for 1 h at 37° C. After 3 washes in PBS-0.05% Tween, mouse monoclonal anti-rabbit IgG (γ-chain specific) alkaline phosphatase conjugate (1:4000 in PBS-BSA 1%, Sigma) was added to each well and incubated for 1 h at 37° C. After 3 washes with PBS-0.05% Tween, bound phosphatase activity was measured with p-nitrophenylphosphate (Sigma) (1 mg/ml in DEA-HCl 1 M bu.er pH 9.8).

ELISA on Recombinant Murine DEC205 Protein

Coding sequence for the N-Terminal part of the murine DEC205, which binds NLDC145 (Srimpton et al., Mol. Immunol., 2009) was purchased from Geneart. The synthetic gene was inserted in the plasmid vector pMT/BiP/V5 (Invitrogen) using BglII and NheI restriction enzymes and a Tween StrepTag was introduced at the C-Terminal end using NheI and XhoI restriction enzymes. The N-Terminal part of murine DEC205 was named (CF14), produced in the Drosophila Scheinder 2 cell line and purified with the (Twin)-Strep-tag kit according to the manufacturer's instructions (Iba Solutions For Life Sciences).

ELISA was performed by standards procedures. Briefly, the 96 flat-bottom wells of microtiter plates (Maxisorp; Nunc, Roskilde, Danemark) were coated overnight at 4° C. with recombinant CF14) at 5 μg/ml in PBS. The plates were washed with PBS, nonspecific binding sites were blocked with PBS containing 4% bovine serum albumin (PBS-4% BSA) for 1 h at 37° C. After 3 washes with PBS, tachyzoites (5 10⁵/well) in PBS were added, centrifuged at 200×g for 5 min and incubated for 1 h30 at 37° C. After 6 washes with PBS-Tween 0.05%, serum from infected T. gondii rabbit (1:400) in PBS-1% BSA was added in each \veil and incubated for 1 h at 37° C. After 3 washes with PBS-0.05% Tween, mouse monoclonal anti-rabbit IgG (7-chain specific) alkaline phosphatase conjugate (1:4000 in PBS-BSA 1%, Sigma) was added to each well and incubated for h at 37° C. After 3 washes with PBS-0.05% Tween, bound phosphatase activity was measured with p-nitrophenylphosphate (Sigma) (1 mg/ml in DEA-HCl 1 M buffer pH 9.8).

Flow Cytometry Analysis

Binding of RH strains to mutuDC cells was determined by flow cytometry. MutuDC 1950 cells (a murine dendritic cell line, DEC205⁺) were cultured as previously described (Fuertes Marraco et al., 2012). Aliquots of 10⁶ mutuDC cells in cold PBS 5% FBS were mixed with 2×10⁶ parasites (MOI 2) and incubated for 1 hour on ice. Unbound parasites were removed by three washes at 100xg for 5 min. The cells transferred in a 96-well plate round bottom, were incubated with an anti-FcγR mAb (clone 2.4G2, eBioscience) to block nonspecific binding and further incubated for 30 min on ice with monoclonal antibody T4 2E12, specific for T. gondii tachyzoite surface glycoprotein, gp23 (Tomavo et al., 1993). Cells were washed with cold PBS 5% FBS and then stained with APC-conjugated anti-mouse IgG for 30 min on ice. The fluorescence intensity of the cells was determined by flow cytometry (MACS Quant, Miltenyi Biotec).

Immunofluorescence Assays

For immunofluorescence assays, MutuDC 1950 cells (a murine dendritic cell line, DEC205+) were cultured as previously described (Fuertes Marraco et al., 2012). MutuDC cells were seeded on coverslips overnight. After washes with PBS, cells were fixed with a 3% paraformaldehyde solution for 30 min Cells were washed 3 times with PBS and saturated during 1 h in PBS-10% FBS, and then incubated with 106 tachyzoites for 30 min at room temperature. Cells were washed 3 times with PBS to eliminate unspecific tachyzoite fixation and incubated with serum from T. gondii infected rabbit (1:100). After washes with PBS, bound tachyzoites were detected using Alexa Fluor 568-conjugated goat anti-rabbit IgG antibodies (1:1,000, Molecular Probes). After washes in PBS, the visualization was carried out using a Leica microscope.

Mice

Twenty-four-week-old female C57BL/6 mice were purchased from CER Janvier (Le Genest Saint Isle, France) and maintained under pathogen-free conditions in the animal house of the University of Tours. Experiments were carried out in accordance with the guideline for animal experimentation (EU Directive 2010/63/EU) and the protocol was approved by the local ethics committee (CEEA VdL).

Toxoplasma gondii Strains (RH-DC2 and RH-DC2-SAG1)

T. gondii strain RH control, RH-DC2 and RH-DC2-SAG1 tachyzoites were produced in HFFs cultured in DMEM (Pan Biotech GmbH) supplemented with 10% of heat-inactivated FCS (Dutscher), 2 mM glutamine (Pan Biotech GmbH), 50 U/ml of penicillin and 50 μ/ml of streptomycin (Pan Biotech GmbH) at 37° C. in 5% CO2 atmosphere. They were harvested during lysis of the host cells by centrifugation at 600 g for 10 min.

Tumor Cells and Tumor Cell Inoculation (EG7)

EG7 cells (EL4-OVA thymoma cells transfected with chicken albumin cDNA) are cultured in Roswell Park Memorial Institute medium (RPMI, Pan Biotech GmbH) with 5×10⁵ M of 2-mercaptoethanol, 50 UI/mL of penicillin and 50 mg/mL of streptomycin. 5×10⁵ live EG7 cells are inoculated subcutaneously in the right flank of mice. Tumor diameters are measured 3 times weekly, and mice are euthanized when tumor diameters reached 25,000 mm³.

T. gondii Administration

Mice are injected subcutaneously in the right flank at day 4 and again at day 7 with 5×10² freshly isolated tachyzoites of RH ctrl and RH-DC2-SAG1 strain of T. gondii.

Tumor Cytokine Measurements

Tumors were cut into small pieces of 2-4 mm and dissociated with Tumor Dissociation Kit and gentleMACS Dissociator (Miltenyi Biotech GmbH, Germany) Following dissociation, cells were pelleted and supernatant were collected for cytokine analysis. Cytokines were quantified in the supernatant using cytometric bead array for IFN-γ, TNF-α, Il-2, Il-4, Il-5, Il-10, Il-17A, IL-23 and GM-CSF (MACSPlex Cytokine 10 kit mouse, Miltenyi Biotec GmbH, Germany), commercial ELISA kits for TGF-β and IL-15 (Invitrogen, Thermo Fisher Scientific, USA) and for IL12p40 and IL-6 (eBioscience).

Cell Populations Phenotyping

Spleen and tumor cell phenotypes were analyzed by flow cytometry. Spleen cells were obtained as described in Example 1. Spleen cells were then pelleted and resuspended in PBS, 2 mM EDTA, 1% FBS for staining and cytometry analysis. Red blood cells from tumor cells obtained as described for cytokine measurements were lysed using red blood cell lysis buffer and cells were resuspended in PBS, 2 mM EDTA, 1% FBS for staining and cytometry analysis. Cells were stained with monoclonal antibodies purchased from Miltenyi Biotec GmbH. The following antibodies were used: REA clones of anti-mouse CD3-APC-Vio770 (REA606), CD4-Vioblue (REA 604), CD8a-PE-Vio770 (REA 601), Foxp3-Vio515 (REA 788), NKp46-APC (REA 815), CD11c-PE (REA 754), CD11b-APC-Vio770 (REA 592), Ly6C-Vioblue (REA 796), Ly6G-PE-Vio770 (REA 526). Foxp3 staining was performed using Foxp3 Staining Buffer Set (Miltenyi Biotech GmbH, Germany) FACS analysis was performed using a Miltenyi 8-color MACS Quant, and data were analyzed using Flowlogic (Milteni Biotech GmbH, Germany).

Results

ScFv-DEC205 Plasmid Constructs

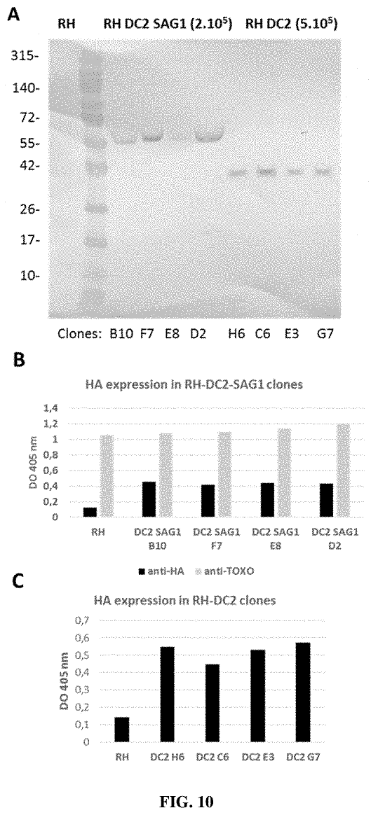

The two constructions of the scFv anti-DEC205 are described in FIGS. 9A-B and below:

As shown in FIG. 9A, the first construction (DC2) encodes the scFv anti-DEC205 protein. In particular, the sequence encoding said DC2 protein included: the Kozak sequence, the ATG, the sequence encoding the N terminal signal sequence of SAG1, the HA tag, the VH region followed by the VL region of anti-DEC205 (scFv anti-DEC205), the linker GGGAS and the sequence encoding the SAG1 anchor signal (GPI) with a stop codon. This sequence is flanked in 5′ by a PmeI site and in 3′ by a NotI site. Then, the T. gondii strain RH tachyzoites were transfected the DC2 construction.

As shown in FIG. 9B, the second construction (DC2 SAG1) encodes the scFv anti-DEC205 protein fused to SAG1. In particular, the sequence encoding said DC2 SAG1 protein included: the Kozak sequence, the ATG, the sequence encoding the N terminal signal sequence of SAG1, the HA tag, the VH region followed by the VL region of anti-DEC205 (scFv anti-DEC205), the linker GGGAS, the sequence encoding a truncated SAG1 (without the N terminal signal sequence and without the SAG1 anchor signal) and the sequence encoding the SAG1 anchor signal (GPI) with a stop codon. This sequence is flanked in 5′ by a PmeI site and in 3′ by a NotI site. Then, the T. gondii strain RH tachyzoites were transfected with the DC2 SAG1 construction.

Cell Surface Expression of scFv-DEC205

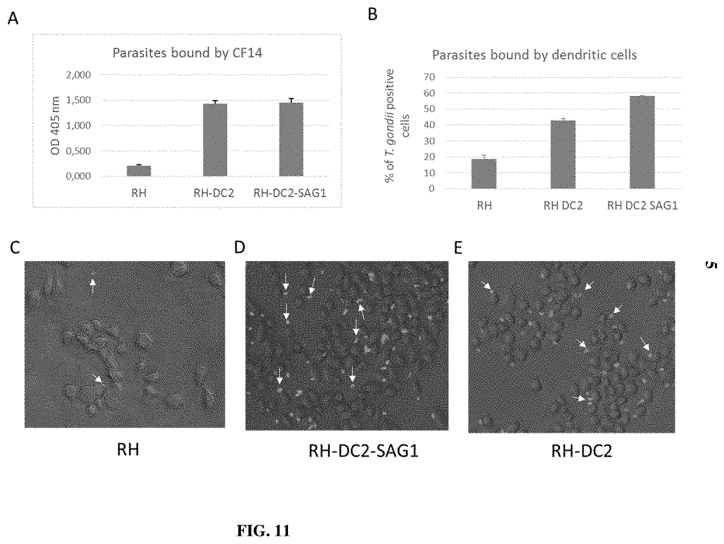

A Western-blot analysis was performed to analyze the expression of the scFv anti-DEC205 (DC2) and the scFv anti-DEC205 fused to SAG1 (DC2 SAG1) proteins in various selected clones of T. gondii (FIG. 10A). Briefly, T. gondii transfected with DC2 or DC2 SAG1 were lysed and probed with anti-Tag HA polyclonal antibodies.

As shown in FIG. 10A, the DC2 and DC2 SAG1 proteins migrate at the expected size under reducing conditions (55 kDa and 42 kDa respectively). Thus, DC2 and DC2 SAG1 proteins are expressed in all T. gondii strains.

An ELISA analysis was further performed on various selected clones of RH-DC2-SAG1 (FIG. 10B) and RH-DC2 (FIG. 10C). Briefly, T. gondii transfected with DC2 or DC2 SAG1 were fixed with glutaraldehyde in flat bottomed wells. The DC2 and DC2 SAG1 proteins were probed with rabbit anti-HA polyclonal antibodies and the parasites were probed with a serum from a T. gondii infected rabbit. Results are expressed as optical density (OD).

As shown in FIG. 10B-C, the scFv anti-DEC205 fused to SAG1 (DC2 SAG1) protein, and the scFv anti-DEC205 (DC2) protein are expressed in all T. gondii strains.

RH-DEC205 Targets the DEC205 Receptor

The ability of RH-DC2 and RH-DC2-SAG1 strains to bind to DEC205 receptor was analyzed by ELISA. The N-Terminal part of the murine DEC205 receptor, which binds NLDC145 (Srimpton et al., Mol. Immunol., 2009) was produced in the Drosophila Scheinder 2 cell line (CF14 recombinant protein) and used to coat flat bottomed wells. Following incubation and washing steps, the bound parasites were probed with a serum from a T. gondii infected rabbit. Results are expressed as optical density (OD).

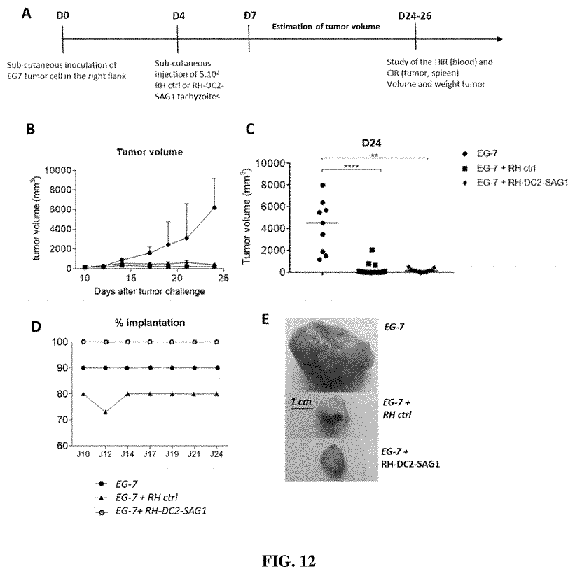

As shown in FIG. 11A, RH-DC2 and RH-DC2 SAG1 bind efficiently to the recombinant DEC205 protein.

RH-DEC205 Targets Dendritic Cells

The ability of RH-DC2 and RH-DC2 SAG1 strains to bind to dendritic cells was analyzed by flow cytometry (FIG. 11B) and immunofluorescence assays (FIGS. 11C, D and E). For flow cytometry analysis, MutuDC 1950 cells (murine dendritic cell line, DEC205⁺) were incubated 1 hour on ice with RH, RH-DC2 or RH-DC2-SAG1 strains respectively (MOI: 2). After washes, bound parasites were stained with a monoclonal antibody specific for T. gondii tachyzoite surface glycoprotein, gp23 followed by APC-conjugated anti-mouse IgG. Results are expressed as percentage of T. gondii positive cells.

As shown in FIG. 11B, cell surface T. gondii expression of scFv anti-DEC205 enhanced the percentage of T. gondii binding to mutuDC cells with a multiplication factor of at least 2.5.

To visualize parasite binding to mutuDC cells, immunofluorescence assays were performed. Briefly, MutuDC 1950 cells (murine dendritic cell line, DEC205±) were seeded on coverslips overnight in culture medium. After washes with PBS, cells were fixed with a 3% paraformaldehyde solution, washed with PBS washed, saturated in PBS-10% FBS, and then incubated with 10⁶ tachyzoites (RH, RH-DC2, RH-DC2-SAG1). After washes to eliminate unspecific tachyzoite fixation, bound tachyzoites were detected using serum from T. gondii infected rabbit followed by Alexa Fluor 568-conjugated goat anti-rabbit IgG. Slides were examined under a Leica microscope.

As shown in FIG. 11C-E, RH-DC2 and RH-DC2-SAG1 strains can bind more efficiently to dendritic cell then RH strain.

RH-DEC-205 Treatment Suppresses and/or Regresses an Established Solid Tumor Development

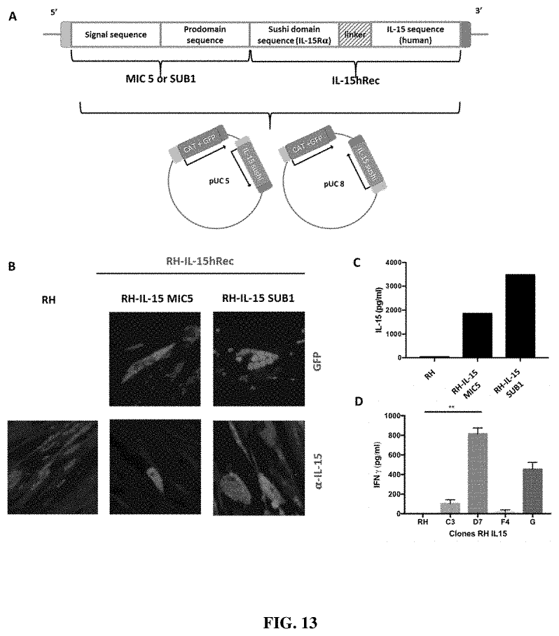

As shown in FIG. 12, treatment with 500 tachyzoites of the RH-DC2-SAG1 strain by sub-cutaneous route decreased tumor (i.e., volume and weight) in mice. Indeed, tumor volume and weight in mice treated with RH or RH-DC2-SAG1 strains was significantly lower than in non-treated mice (FIGS. 12B-C and E).

Interestingly, a significant reduction of tumor implantation was observed in mice treated with RH-DC2-SAG1 (FIG. 12D).

All these results suggest that a sub-cutaneous injection of RH-DC2-SAG1 tachyzoites exhibited good efficacy against tumor development.

RH-DC2 SAG1 Induces a Protective Immune Response Against Tumor Development

MIC1-3 KO is a mutant strain of T. gondii RH lacking the mic1 and mic3 genes. Disruption of these two genes impairs virulence in mice.

a) Tumor Compartment

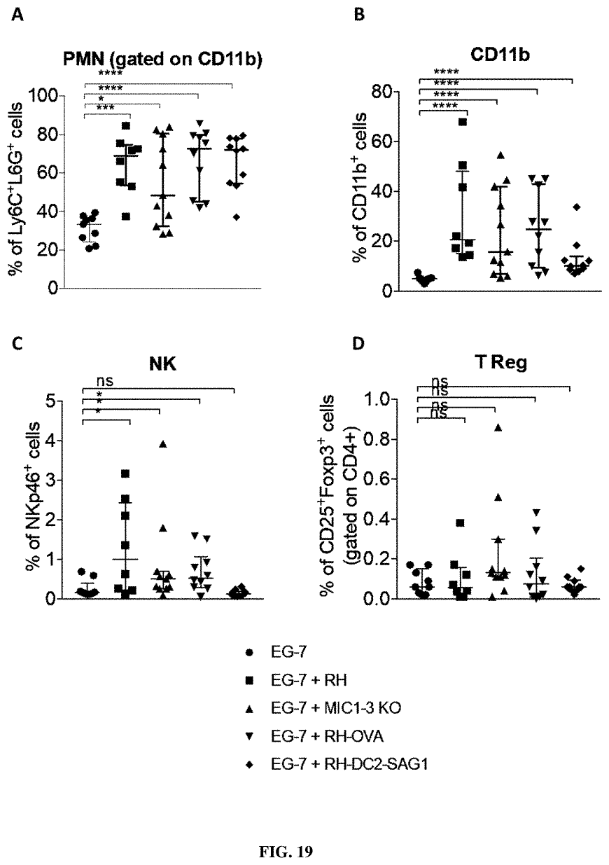

As described in FIG. 18A-C, RH-DC2 SAG1 treatment induced significant increase of secretion of different cytokines (IL12p40, IL-15 and IL-6) in comparison to cytokine secretion by tumor from EG-7-mice.

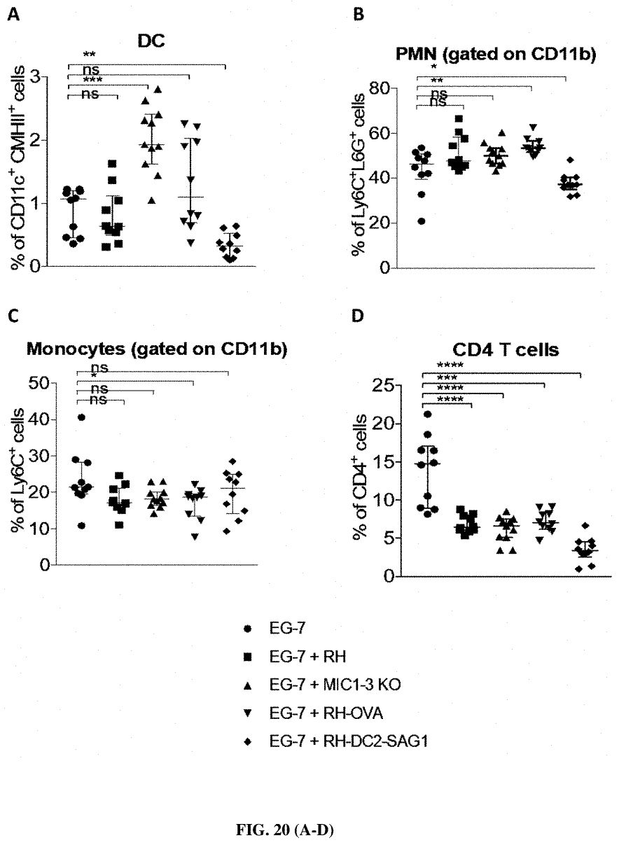

Concerning tumor myeloid cell sub-populations, a significant increase of Ly6C+Ly6G % (PMN) and CD11b+(monocytes) was observed for the four T. gondii treated groups compared to the tumor EG7 mice (FIG. 19A-B).

For tumor lymphoid cell sub-populations, an increase of NK cell population (NKp46+ cells) was observed for RH, MIC1-3KO and RH-OVA treated groups (FIG. 19C), but no significant difference is observed between RH-DC2 SAG1 treated group and tumor EG7 mice. No significant difference was observed between the four T. gondii treated groups and tumor EG7 mice for Treg cells (Foxp3+) (FIG. 19D).

b) Systemic Compartment (i.e., Spleen)

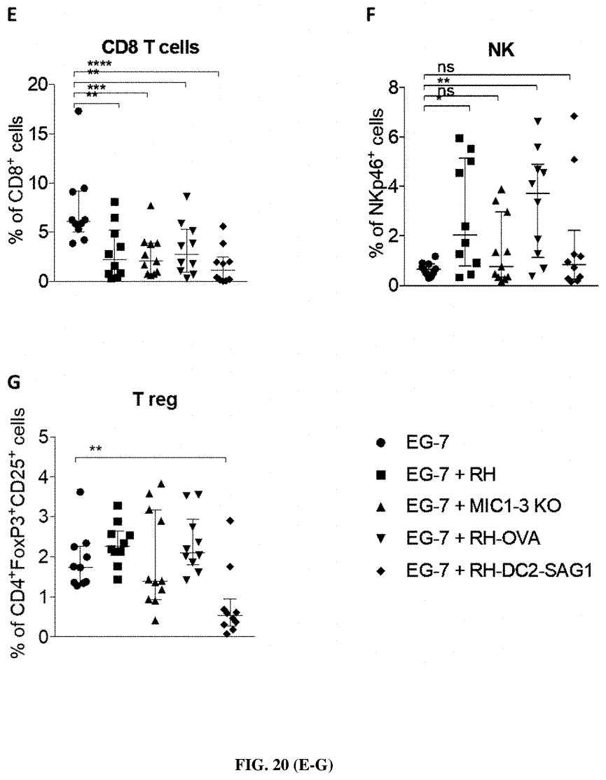

Concerning spleen myeloid cell sub-populations, a significant increase of CD11c+/CMH II+ cells (dendritic cells) was observed for the RH K01-3 treated group, a significant decrease for the RH DC2 SAG1 treated group, while no significant differences were observed for both RH and RH-OVA treated groups, compared to the tumor EG7 mice (FIG. 20A). For Ly6C+Ly6G % cells (PMN), a significant increase was observed for the RH-OVA treated group, a significant decrease for the RH DC2 SAG1 treated group, while no significant differences were observed for both RH and RH K01-3 treated groups, compared to the tumor EG7 mice (FIG. 20B). No significant differences are observed between the four T. gondii treated groups and tumor EG7 mice for CD11b+(monocytes) (FIG. 20C).

For spleen lymphoid cell sub-populations, a significant decrease of CD4+ and CD8+ T cells was observed for the four T. gondii treated groups compared to the tumor EG7 mice (FIG. 20D-E). A significant increase of NK cell population (NKp46+ cells) was observed for both RH and RH-OVA treated groups compared to tumor EG7 mice. No significant differences were observed for RH K01-3 and RH-DC2 SAG1 groups, compared to tumor EG7 mice (FIG. 20F).

A decrease of Treg cells (Foxp3+) was observed for RH-DC2 SAG1 T. gondii treated group, compared to tumor EG7 mice. No significant differences were observed for RH, RH K01-3 and RH-OVA treated groups, compared to the tumor EG7 mice (FIG. 20G).

### Example 3: Specific Secretion of Immunotherapeutic Cytokine by the Strain and in Vivo Effects

Materials and Methods

Parasites

N. caninum (NC-1 strain) tachyzoites were grown by continuous passage in confluent human foreskin fibroblasts (HFFs) in Dulbecco's minimal medium (DMEM, Pan Biotech GmbH) supplemented with 10% of heat-inactivated fetal calf serum (FCS, Dutscher), 2 mM glutamine (Pan Biotech GmbH), 50 U/ml of penicillin/50 μ/ml of streptomycin (Pan Biotech GmbH) and 1% HEPES (Invitrogen) at 37° C. in 5% CO2 atmosphere. For subsequent experiments, infected cultures at the stationary growth phase were scraped and then passed several times through a 27-gauge needle (Millipore, Billerica, USA). The N. caninum tachyzoites were collected, during lysis of the host cells, by centrifugation at 600 g for 10 min.

T. gondii strain RH tachyzoites were produced in HFFs cultured in DMEM (Pan Biotech GmbH) supplemented with 10% of heat-inactivated FCS (Dutscher), 2 mM glutamine (Pan Biotech GmbH), 50 U/ml of penicillin and 50 μ/ml of streptomycin (Pan Biotech GmbH) at 37° C. in 5% CO2 atmosphere. They were harvested during lysis of the host cells by centrifugation at 600 g for 10 min.

Plasmid Construction of the RH-IL-15hRec

The plasmid pUC8 CAT/GFP-IL-15hRec (human IL-15/IL-15Rα sushi) was used to construct the recombinant RH-IL-15hRec and NC1-IL15hRec.

pUC8 CAT/GFP-IL-15hRec is a pUC8 plasmid in which the sequence encoding the complex human IL-15/IL-15Rα sushi (IL-15hRec) including the N-terminal signal sequence and the prodomain motif of SUB1 or MICS is cloned in the expression cassette between PmeI and NotI sites.

pUC8 contains two expression cassettes. One was designed to express a CAT-GFP fusion protein to allow drug selection of stably transfected parasites (cassette CAT-GFP), the second was designed to express proteins of interest. The sequence encoding the protein of interest must include an ATG and a stop codon. The expression of CAT-GFP is driven by the promoter of the T. gondii α-tubulin gene (αTUB5) and the 3′ untranslated region (3′UTR) of the T. gondii SAG1 gene. This expression cassette is bordered in the 3′ position and 5′ position by Loxp sites. These LoxP sites were added to suppress the cassette CAT-GFP from DNA genome of the parasite by the use of a Cre recombinase which recognizes specifically these sites.

The expression of the protein of interest is driven by the promoter of the T. gondii α-tubulin gene (αTUB5) in which a five-repeat element was inserted upstream of the transcriptional start site (leading to promoter αTUB8) for high-level expression of the protein (Soldati et al., 1995 PMCID: PMC231911). The sequence of the protein of interest is cloned in PmeI/NotI sites.

Obtention of pUC8

Generation of the plasmid pCN1, containing a cassette to express the fusion protein SAG1-GFP driven by the promoter of the T. gondii α-tubulin gene (αTUB5) and the 3′ untranslated region of the T. gondii SAG1 gene (3′UTR SAG1). The sequence encoding SAG1 (including the signal sequence, without the GPI anchor signal sequence) was amplified by PCR by using plasmid pcDNA3-SAG1 as the template (Mévélec et al., 2005 D0110.1016/j.vaccine.2005.04.025) and the primer sequences GGTTTTGACGTCACCATGTTTCCGAAGGCAGTG (SEQ ID NO: 53) (AaTII, underlined) and TTGCTCACCATCCTAGGTGCAGCCCCGGCAAA (SEQ ID NO: 54) (AvrII, underlined). The sequence encoding GFP was amplified by PCR by using plasmid pmic3-GFP (Striepen et al., 1998 D0110.1016/50166-6851(00)00379-0) and the primer sequences TTTGCCGGGGCTGCACCTAGGATGGTGAGCAA (SEQ ID NO: 36) (AvrII, underlined) and

(SEQ ID NO: 37)
CGGTGATTAATTAATCGAGCGGGTCCTGGTTCG

(PacI, underlined). SAG1 and GFP PCR products were digested by AaTII/AvrII and AvRII/PacI respectively and ligated into plasmid pT230TUB/Ble (Kim et al., 1993 PMID: 8235614) previously digested with AaTII/PacI. In the resulting plasmid (pCN1), the sequence encoding BLE is replaced by the sequence encoding the secreted fusion protein SAG1-GFP under the control of promoter αTUB5.

Generation of the plasmid pCN5, containing a cassette expressing CAT under the promoter αTUB5, bordered by the Loxp sites in the 3′ position and 5′ position. The sequence of the cassette expressing CAT, driven by the promoter of the T. gondii a-tubulin gene (α-TUB5) and 3′ untranslated region of the T. gondii SAG1 gene (3′UTR SAG1), was amplified by PCR by using pmic3KO-2 (Cérède et al., 2005 DOI:10.1084/jem.20041672) as the template and the primer sequences GCGGCCAAGCTTATAACTTCGTATAATGTATGCTATACGAAGTTATGATATG CATGTCCGCgttcgtgaaatctctgatcaagcgg (SEQ ID NO: 38) (including HindIII and Loxp sequences, underlined and in italic respectively) and cgacgcacgctgtcactcaacttgctGCTAGAACTAGTGGATCCATAACTTCGTATAGCATA CATTATACGAAGTTATCCCTCGG (SEQ ID NO: 39) (including SpeI and Loxp sequences, underlined and in italic respectively).

The PCR fragment was digested by HindIII/SpeI and cloned into pCN1 which has been previously digested by the same enzymes HindIII/SpeI. In the resulting plasmid (pCN5), the cassette expressing the fusion protein SAG1-GFP under the control of promoter αTUB5 is replaced by the cassette expressing CAT under the promoter αTUB5, bordered by the Loxp sites in the 3′ position and 5′ position.

Generation of the plasmid pUC18 CAT-GFP containing a cassette to express the fusion protein CAT-GFP driven by the promoter of the T. gondii α-tubulin gene (αTUB5) and the 3′ untranslated region of the T T. gondii SAG1 gene (3′ UTR SAG1). The cassette is bordered on both sides by Loxp sites.

To clone the sequence encoding GFP in fusion with the sequence encoding CAT, a fragment including LoxP (5′ position), promoter αTUB5 and the sequence encoding CAT without stop codon but with an AvrII site to clone GFP in fusion with CAT, was amplified by PCR by using pCN5 as the template and the primer sequences GTATCGATAAGCTTATAACTC (SEQ ID NO: 40) (HindIII underlined) and CACAACGGTGATTAACCTAGGAGCCCCGCCCTG (SEQ ID NO: 41) (AvrII, underlined). The amplified fragment was digested by HindIII/AvrII. The sequence encoding GFP was obtained from pCN1 by digestion with AvrII/PacI. The plasmid pCN5 was digested by HindIII/PacI to eliminate the HindIII/PacI fragment corresponding to LoxP (5′ position), promoter αTUB5 and the sequence encoding CAT. Finally, the three fragments were ligated to obtain a recombinant plasmid containing the cassette expressing CAT-GFP under the promoter αTUB5, bordered by the Loxp sites in the 3′ position and 5′ position. This cassette was further cloned into pUC18 using HindIII and XbaI to obtain pUC18 CAT-GFP. Generation of pUC8, containing one cassette, bordered by LoxP sites, to express the fusion protein CAT-GFP driven by the promotor of the T. gondii α-tubulin gene (αTUB5) and the 3′ untranslated region of the T. gondii SAG1 gene (3′UTR SAG1) and another one to express a protein of interest driven by a modified promotor of the T. gondii α-tubulin gene (αTUB8) and the 3′ untranslated region of the T. gondii SAG1 gene (3′UTR SAG1). In pUC8 the two cassettes are in opposite orientation.

The expression cassette with a modified promoter of the T. gondii α-tubulin gene (αTUB8) was obtained by addition of five repeat sequences (Soldati et al., 1995), 70 bases upstream the major transcription start site in the T. gondii α-tubulin gene (αTUB5). Plasmid pT230TUB/Ble was used to obtain the promoter αTUB8 and the 3′UTR SAG1 sequences. Two enzymes restriction sites PmeI and NotI were included between the αTUB8 and 3′UTR SAG1 sequences to allow insertion of the sequence encoding the protein of interest. The XbaI restriction sites, located at both end of the expression cassette, were used to clone this expression cassette in pUC18 CAT-GFP in both orientations. The resulting plasmid was pUC8 with the two cassettes in the opposite orientation.

Generation of Plasmid pUC8 CAT/GFP-IL-15hRec

pUC8 CAT/GFP-IL-15hRec is a pUC8 plasmid in which the sequence encoding the complex IL-15/IL-15Rα sushi (IL-15hRec) including the N-terminal signal sequence and the prodomain motif of SUB1 or MICS is cloned in the expression cassette between PmeI and NotI sites. The complex is expressed under the control of αTUB8.

Engineering of Human Recombinant Cytokine IL-15 (IL-15hRec)

Il-15hRec results from the association of the human sushi domain of the human IL-15 receptor subunit alpha precursor (SEQ ID NO: 3 or 4) with the human mature protein IL-15 (SEQ ID NO: 5 or 6) via a peptide linker (SEQ ID NO: 7 or 8). All these sequences were also optimized for protozoan organism (https://eu.idtdna.com/CodonOpt). Because IL-15hRec is destined for regulated secretion by T. gondii or N. caninum, it is synthesized as preproprotein with an N-terminal signal peptide (SEQ ID NO: 9, 10, 13 or 14) and a separate, cleavable prosequence or prodomain (SEQ ID NO: 11, 12, 15 or 16) necessary for trafficking to the micronemes. We have engineered two possible secretion pathways with i) proDomain TgSUB1 (a micronemal subtilisin-like serine protease) or with ii) proDomain MICS (a small and soluble micronemal protein). In each case, the proDomain is critical for correct folding as well as progression in the secretory pathway. Kozak sequence comprising start codon ATG (Seeber et al., 1997 DOI10.1007/s004360050254) and stop codon (TAA) were included. Finally, in upstream and in downstream of the sequence, two restriction sites were added as respectively: PmeI (GTTTAAAC) and NotI (GCGGCCG). The synthetic gene SUB1IL-15hRec or MICSIL-15hRec was inserted into the pUC8 plasmid with restriction sites PmeI and NotI.

All basic molecular biology procedures were carried out as described by Sambrook and Russel (Sambrook J., R. D. W. (2001) in Molecular Cloning: A Laboratory Manual (Sambrook, J., and Russel, D. W., eds) Cold Spring Harbor Laboratory Press, Cold Spring Harbor, N.Y.). Taq polymerase, restriction enzymes, calf intestinal phosphatase and T4 DNA ligase were from Promega or New England Biolabs.

The resulting plasmid pUC8 CAT/GFP-IL-15hRec expresses the secreted complex IL-15/IL-15Rα sushi (IL-15hRec) under the control of promotor αTUB8 and the fusion protein CAT-GFP under the control of promotor αTUB5 to allow drug selection of stably transfected parasites.

Production of Human Recombinant Cytokine IL-15 (IL-15hRec) by N. caninum or T. gondii

Transfections were performed with 10⁷ N. caninum or T. gondii tachyzoites in a volume of 650 μl of cytomix (Van den Hoff et al., Nucleic Acids Res. 1992 Jun. 11; 20(11): 2902) containing 3 mM ATP and 3 mM gluthatione and 50 μg of purified plasmid DNA (the plasmids were purified using the Qiagen Kit®) linearized with PciI or 100 μg of purified plasmid DNA (the plasmids were purified using the Qiagen Kit®) non linearized. Electroporations were performed in disposable cuvettes (4 mm gap) with an electroporator Biorad (electroporation settings: 2000 V, 50 ohms, 25 mF). After electroporation, the parasites were kept in the hood for 15 min at room temperature and then transferred to a fresh culture of fibroblast monolayers (25 cm2 flask). After 24 hours the parasites N. caninum and T. gondii were respectively subjected to 80 μM and 40 μM of chloramphenicol selection. After 10 to 15 days of selection, the parasites were cloned by limiting dilution in the wells of a 96-well plate of HFF cells in the presence of selection agent and the clones were amplified.

Immunofluorescence Assays

HFF monolayers on glass coverslips were infected with NC-IL-15hRec or RH-IL-15hRec for 24 to 48 h. The cells were then fixed, permeabilized and blocked. Infected cells were first washed two times in PBS and fixed in PBS plus 4% paraformaldehyde for 30 min at room temperature. Then the coverslips were washed thrice in PBS and were permeabilized in PBS supplemented with 0.2% Triton X-100 for 20 min at room temperature. At this concentration and incubation time, triton X-100 permeabilizes membranes of the infected host cell, including the membrane of the parasitophorous vacuole and the parasite plasma membrane. After three washes the coverslips were blocked in PBS plus 1% bovine serum albumin (BSA) for 1 h at room temperature. Fixed cells were labeled with IL-15 polyclonal antibody (PA5-34511 ThermoFischer Scientific, 10 μg/mL in PBS/BSA 0.2%) over night at 4° C. in humid chamber. After three washes in PBS, the samples were incubated for 45 min with secondary Alexa Fluor 568-conjugated goat anti-rabbit IgG antibody (A-11036 ThermoFisher Scientific, 1/1000 dilution in PBS/BSA 0.2%) at room temperature and in a humid chamber. Microscope slides were mounted on glass slides using Immu-Mount (Thermo Scientific) and the visualization was carried out using a Leica microscope. Image analysis was performed with NIH ImageJ software.

ELISA: IL-15hRec

The secretion in the culture supernatant of human recombinant cytokine IL-15 has been assessed using enzyme-linked immunosorbent assay (ELISA) method. Briefly and according to the manufacturer's instructions (DY6924 RD SYSTEMS), Capture Antibody was coated in a 96-well plates at 4 μg/mL and incubated overnight at 4° C. The wells were saturated with Reagent Diluent for a minimum of 60 min at room temperature and washed with PBS-Tween 0.05% prior to incubation with standard at increasing concentrations (ranging from 0 to 4000 pg/mL) and diluted samples ( 1/10 and 1/50 in Reagent Diluent) overnight at 4° C. Wells were then washed, which was repeated after each of the following incubations: Detection Antibody for 2 h at room temperature, Streptavidin-HRP for 20 min at room temperature and finally Substrate Solution for 20 at room temperature avoiding placing the plate in direct light. Enzymatic reactions were stopped with the Stop Solution and the absorbance was measured at 450 nm using a microplate reader (Biotek). Wells coloration correlated to the presence of secreted IL-15hRec in the culture supernatant and the absorbance at 450 nm was then proportional to IL-15hRec content.

Cytokine Production

Splenocytes from naïve mice were recovered and purified as described (Rhode et al., 2016 DOI: 10.1158/2326-6066.CIR-15-0093-T). Briefly, single-cell splenocyte suspensions were obtained from spleen first pressed and then filtered through a nylon mesh. Hypotonic shock (0.155 M NH₄Cl, pH 7.4) was used to remove splenic erythrocytes. The splenocytes were stimulated for 72 h with IL15hRec containing supernatants diluted twice. The cells (5×10⁵) were seeded into 24-well plates in 1 ml RPMI 1640 containing 5% FCS, and supernatants were collected 72 h after activation. IFNγ was quantified by ELISA using ready-set-go kit (ebioscience).

Cytokine Measurement and Cell Populations Phenotyping after NC1-IL15hRec Infection

Briefly, supernatants of culture of the engineered NC1-IL15hRec strain and NC1 strain were dosed for IL-15 48 h after infection (MOI 0.2). Mouse splenocytes were infected with NC1-IL15hRec (MOI 1) and IFN-γ was dosed in the supernatant after 48 h.

Human PBMCs were infected with NC1-IL15hRec or NC1 (MOI 1), and supernatants of culture were dosed by ELISA 24 h after infection. At the same time point, cells were analyzed by flow cytometry.

Mice

Eight week-old female inbred C57BL/6 mice were purchased from CER Janvier (Le Genest Saint Isle, France) and maintained under pathogen-free conditions in the animal house of the University of Tours. Experiments were carried out in accordance with the guideline for animal experimentation (EU Directive 2010/63/EU) and the protocol was approved by the local ethics committee (CEEA VdL).

Tumor Cells and Tumor Cell Inoculation (EG7)

EG7 cells (EL4-OVA thymoma cells transfected with chicken albumin cDNA) are cultured in Roswell Park Memorial Institute medium (RPMI, Pan Biotech GmbH) with 5×10⁵ M of 2-mercaptoethanol, 50 UI/mL of penicillin and 50 mg/mL of streptomycin.

5×10⁵ live EG7 cells are inoculated subcutaneously in the right flank of mice. Tumor diameters are measured 3 times weekly, and mice are euthanized when tumor diameters reached 25,000 mm³.

N. caninum Administration

Mice are injected subcutaneously in the right flank at day 4 and again at day 7 with 5×10⁶ freshly isolated tachyzoites of NC1 ctrl and NC1-IL-15hRec strain of N. caninum.

Results

IL-15hRec Plasmid Constructs

Both T. gondii (RH) and N. caninum (NC) strains are engineered to express and secrete a human IL-15/IL-15Rα sushi (IL-15hRec) cytokine. IL-15hRec sushi stands for recombinant human interleukin 15 covalently linked to sushi domain of the human IL-15 receptor alpha. It is composed of the sushi domain (amino acids 117 to 182) of the human alpha receptor, a peptide linker and the human interleukin-15. The sushi domain plays a role of chaperone protein, stabilizes and increases IL-15 activity (Desbois et al., J Immunol. Jul. 1, 2016, 197 (1) 168-178). The complex formed by IL-15 and the sushi domain of IL-15Rα generates a more potent ligand compared to the cytokine alone.

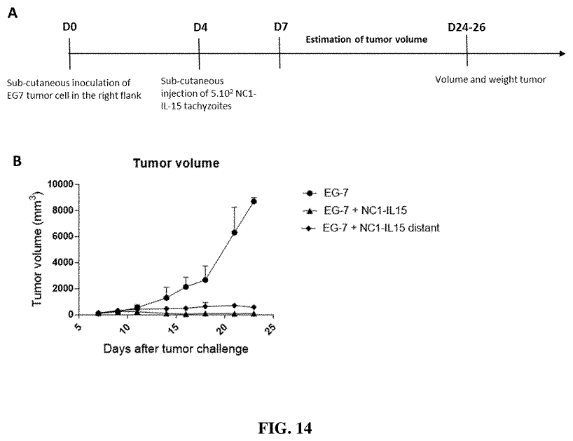

The construction of IL-15hRec is described in FIG. 13A and below:

The IL-15hRec construction encodes for the expression cassettes CAT/GFP and human IL-15/IL-15Rα sushi complex. In particular, the sequence encoding said IL-15hRec protein included: the locations of the preprotein sequence (Signal sequence and Prodomain sequence of MICS or SUB1), human sushi domain of the human IL-15 receptor subunit alpha sequence (Sushi domain sequence (IL-15Rα), linker sequence (grey hatched diagram), and human mature IL-15 (human IL-15 sequence). The light and dark grey blocks represent respectively: restriction site PmeI and restriction site NotI. Then, the T. gondii (RH) and N. caninum (NC) strains tachyzoites were transfected with the IL-15hRec construction.

Of note, according to signal sequence and prodomain of T. gondii used for the plasmid construction (MICS or SUB1), two different strains RH-IL-15 MICS or RH-IL-15 SUB1 are respectively obtained.

IL-15hRec Expression by the Recombinant RH-IL-15 Strains

The expression of GFP and IL-15hRec into transfected RH parasites was analyzed by immunofluorescence assays (FIG. 13B). Briefly, the CAT-GFP cassette construct was detected by fluorescence of the GFP protein, whereas the IL-15hRec cassette construct was detected with an IL-15 antibody.

As shown in FIG. 13B, T. gondii strain RH tachyzoites transfected with plasmid pUC8 CAT/GFP-IL-15hRec express in their cytoplasm the both IL-15hRec and GFP when parasites are contained in the parasitophorous vacuole. In FIG. 13C, IL-15hRec secretion from extracellular T. gondii was analyzed by ELISA method. The culture supernatants from extracellular parasites of RH-IL-15 MICS and RH-IL-15 SUB strains were harvested and the production of IL-15hRec was measured. Extracellular parasites spontaneously secreted Il-15hRec in the medium. In the same manner, secretion of IL-15hRec was observed from parasite-infected cells (data not shown) although we didn't know whether the IL-15hRec production from the infected cells is due to the exocytosis machinery, the rest of the extracellular parasite in cultures after washing of cells, or disruption of host cells and parasitophorous vacuole.

Finally, as shown in FIG. 13D, transgenic parasites could produce biologically active human IL-15. Indeed, immunostimulatory effects of IL-15hRec on murine immune cells were measured by IFNγ quantification according to ELISA method. Incubation with soluble IL-15hRec (from 4 different RH-IL15 clones) for 3 days resulted in elevated IFNγ release by mouse splenocytes.

The IL-15hRec constructions used for T. gondii were also used for N. caninium.

NC-IL-15 Treatment Suppresses and/or Regresses an Established Solid Tumor Development

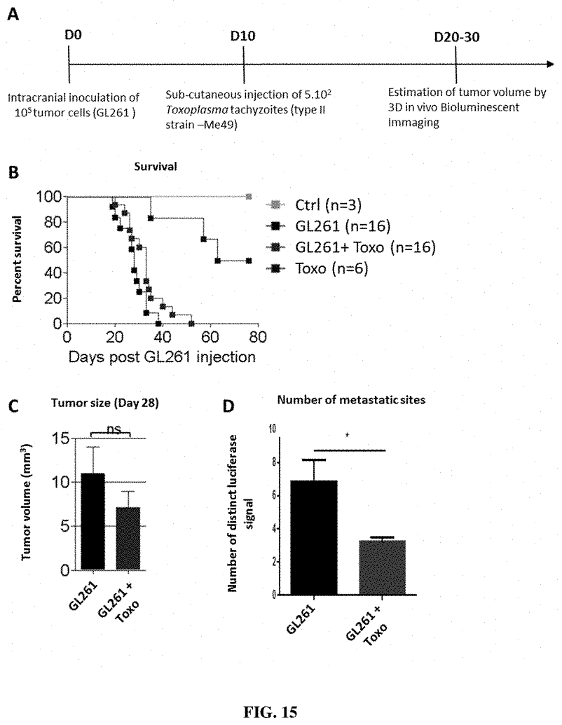

As shown in FIG. 14, treatment with 500 tachyzoites of the NC-IL-15 strain by sub-cutaneous route decreased tumor volume in mice. Indeed, tumor volume and weight in mice treated with NC-IL-15 strains was significantly lower than in non-treated mice (FIG. 14B).

Interestingly, the reduction of the tumor volume was also observed in mice for which subcutaneous injection of NC-IL-15 tachyzoites was performed “at distance” of the tumor site.

These results suggest that sub-cutaneous injection of NC-IL-15 tachyzoites exhibited good efficacy against tumor development.

NC-IL-15 Induces a Protective Immune Response Against Tumor Development

The inventors demonstrated that N. caninum was able to induce tumor regression, notably through an immune cell activation and associated cytokine increased secretion, reprogramming of the tumor microenvironment and direct oncolytic effect. However, improving these protective effects seems essential in order to obtain a total protection in advanced or refractory tumors. To this aim, they engineered a N. caninum strain able to secrete the human IL-15, associated with its sushi domain (alpha subunit of the IL-15 receptor), increasing its stability, binding and biological abilities, to strongly induce the expansion of Th1 associated lymphocyte subsets and prevent their apoptosis in vivo.

They first assessed that the engineered clones were able to secrete a biologically active form of the cytokine. They showed that supernatant of culture of NC1-IL15hRec were able to induce IFN-γ secretion by mouse splenocytes in vitro (the human IL-15 being cross reactive with mice cells, or able to stimulate mouse cells), 4 to 5 times as compared to supernatant from WT Neospora. The NC1-IL15hRec strain was thus able to secrete a functional IL-15 in its environment.

The effect of the NC1-IL15hRec strain on human cells was then tested. Human PBMCs were infected (MOI 1) with NC-1 and NC1-IL15hRec. After 24 hours, the levels of IL-15 were measured in the supernatant, which was only detectable in the wells treated with NC1-IL15hRec. Then, as the recombinant IL-15, the NC1-IL15hRec strain was able to induce the proliferation of human NK cells as shown by the increase of Ki67 expression by human NK cells. Meanwhile, WT N. caninum displayed a much lower increase of proliferation by NK cells. Moreover, while recombinant IL-15 was not able to induce IFN-γ secretion by human PBMCs, but only their proliferation, NC1-IL15hRec induced a strong IFN-γ secretion by human PBMCs, much more importantly than WT N. caninum.

These data clearly demonstrated the plus-value potential of IL-15-armed strain of N. caninum that might strengthen the already important immunomodulatory properties of the WT protozoan parasite.

### Example 4: Toxoplasma Gondii Treatment Suppresses and/or Regresses an Established Glioblastoma Development

Materials and Methods

Mice

Sixteen-week-old female inbred albino C57BL/6 mice were purchased from CER Janvier (Le Genest Saint Isle, France) and maintained under pathogen-free conditions in the animal house of the University of Tours. Experiments were carried out in accordance with the guideline for animal experimentation (EU Directive 2010/63/EU) and the protocol was approved by the local ethics committee (CEEA VdL).

Toxoplasma gondii Strains (Me49)

Tachyzoites of the Me49 strain of Toxoplasma gondii were harvested from infected human foreskin fibroblasts HFF (ATCC Hs27) cultured in monolayers in DMEM, supplemented with 10% heat-inactivated FCS, 50 U/ml penicillin/50 μg/ml 20 streptomycin, and 1% HEPES. Toxoplasma gondii tachyzoites are harvested when monolayers of HFF were completely lysed.

Tumor Cells (GL261)

GL261-Luc cell line was a generous gift of Stéphan Birklé (Institut de Recherche en Cancérologie Nantes-Atlantique UMR S 1232 Centre de Recherche en Cancérologie et Immunologic Nantes-Angers). Cells were grown in DMEM containing 10% fetal calf serum and 1% penicillin/streptomycin. The GL261-Luc murine model is one of the most extensively used for preclinical testing of immunotherapeutic approaches for GBM.

Tumor Cell Inoculation

To establish syngeneic gliomas, GL261-Luc cells (10⁵ cells in 2.5 μL) were intracranially implanted in the brains of albino C57BL/6 mice.

All mice were given subcutaneous buprenorphine injections (0.1 mg/kg) as pre-emptive analgesia. Mice under deep anesthesia after inhalation of isoflurane gas in a nose cone adapted for the stereotaxic frame to maintain an appropriate level of anesthesia were prepared for surgery. After a midline scalp and periosteum incision with lidocaine local anesthesia, tumor cells were stereotactically implanted in the right frontal lobe with a 26 gauge needle into the following coordinates: at a depth of 3 mm, 2 mm lateral to midline and 0.5 mm anterior to bregma.

Animals were monitored daily for any neurological change, weight loss and for their ability to freely access food and water.

Tumor burden was monitored by luciferase imaging on day 7 after implantation. Mice were randomly allocated into treatment arms based on tumor radiance, so that the average tumor radiance in each group was roughly equivalent.

When mice showed predetermined signs of neurologic deficits (failure to ambulate, weight loss >20% mass, lethargy, hunched posture), they were killed.

Toxoplasma gondii Administration

Eleven days after GL261-Luc cell implantation mice received by sub-cutaneous route 500 tachyzoites of Me49 strain (type II) in 50μl.

Results

Evaluation of the number of metastatic sites by 3D in vivo bioluminescent imaging was performed at days 1, 22, 24, 28, 31 and 39.

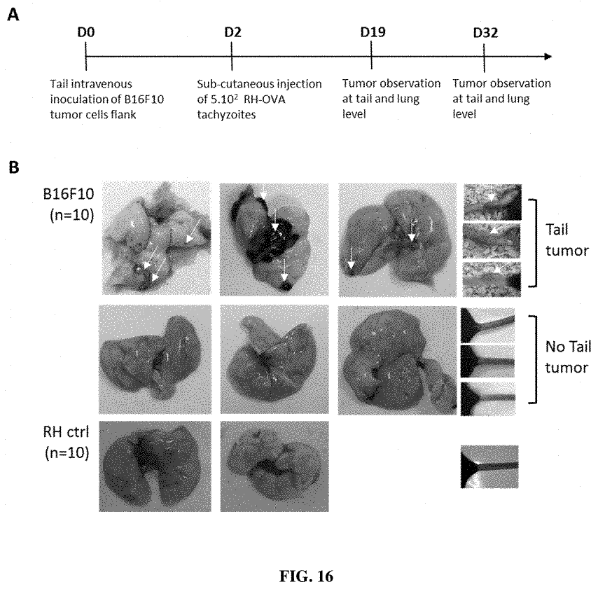

As shown in FIG. 15, treatment with 500 tachyzoites of the Me49 strain by sub-cutaneous route increased survival of mice. Indeed, median survival for GL261 tumor bearing mice was 28 days while for Toxoplasma GL261 tumor bearing mice was 35 days (FIG. 15A).

Moreover, in vivo bioluminescent imaging revealed at D28 that GL261 tumor mice exhibited highly tumor volume (11 mm³). On the other hand tumors from mice treated with T. gondii were less invasive (7 mm³) (FIG. 15B). Furthermore, the number of distinct metastatic sites were significantly lower in the Toxoplasma-treated mice that for non-treated mice (FIG. 15C).

All these results suggest that a single sub-cutaneous injection of Toxoplasma gondii exhibited good efficacy against glioblastoma development.

### Example 5: Recombinant RH-OVA Treatment Suppresses and/or Regresses an Established Lung Melanoma Development in Mice

Materials and Methods

Mice

Twenty-four-week-old female C57BL/6 mice were purchased from CER Janvier (Le Genest Saint Isle, France) and maintained under pathogen-free conditions in the animal house of the University of Tours. Experiments were carried out in accordance with the guideline for animal experimentation (EU Directive 2010/63/EU) and the protocol was approved by the local ethics committee (CEEA VdL).

Toxoplasma gondii Strains (RH-OVA)

T. gondii strain RH-OVA tachyzoites were produced in HFFs cultured in DMEM (Pan Biotech GmbH) supplemented with 10% of heat-inactivated FCS (Dutscher), 2 mM glutamine (Pan Biotech GmbH), 50 U/ml of penicillin and 50 μ/ml of streptomycin (Pan Biotech GmbH) at 37° C. in 5% CO2 atmosphere. They were harvested during lysis of the host cells by centrifugation at 600 g for 10 min.

Tumor Cells (B16F10)

B16F10 murine melanoma cell line were cultured at 37° C. in 5% CO2 atmosphere in complete media consisting of RPMI 1640, 2 mM 1-glutamine, 100 U ml⁻¹ penicillin and 100 mg ml⁻¹ streptomycin and 10% fetal bovine serum.

Tumor Cell Inoculation

For tumor inoculation, mice were challenged with 10⁶ B16/F10 melanoma cells by tail venous injection. Development of subcutaneous tumor was observed daily. Lung metastasis were observed on mice at day 19 and day 32 post implantation.

Toxoplasma gondii Administration

Mice are injected subcutaneously in the right flank at day 2 with 5×10² freshly isolated tachyzoites of RH-OVA strain of T. gondii.

Results

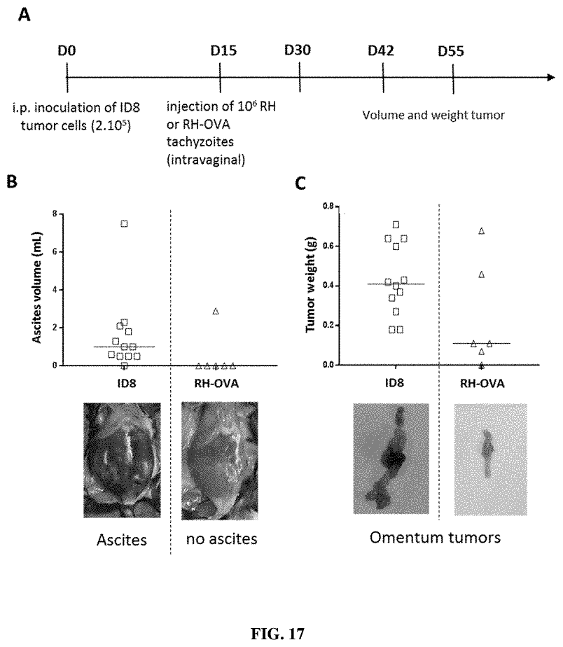

As shown in FIG. 16B, Toxoplasma treatment inhibited drastically subcutaneous tumor growth compared to control at day 19 and 32 after tumor challenge. Subcutaneous tumor growth of control mice was rapid for all mice, while tumors of T. gondii vaccinated mice were absent.

These results clearly demonstrate that Toxoplasma gondii injection inhibit not only growth of the tumor implanted into the dermis, but also the development of lung metastasis.

### Example 6: Recombinant RH-OVA Strain for Treating Ovarian Cancer in Mice

Materials and Methods

Mice

Twenty-four-week-old female C57BL/6 mice were purchased from CER Janvier (Le Genest Saint Isle, France) and maintained under pathogen-free conditions in the animal house of the University of Tours. Experiments were carried out in accordance with the guideline for animal experimentation (EU Directive 2010/63/EU) and the protocol was approved by the local ethics committee (CEEA VdL). Toxoplasma gondii strains (RH-OVA)

Tumor Cells (ID8)

ID8-Luc ovarian carcinoma cell line was cultured at 37° C. in 5% CO2 atmosphere in complete media consisting of RPMI 1640, 2 mM 1-glutamine, 100 U ml⁻¹ penicillin and 100 mg ml⁻¹ streptomycin and 10% fetal bovine serum. Cells were grown in DMEM supplemented with 5% FBS and 1×insulin-transferrin-sodium selenite media supplement.

Tumor Cell Inoculation

For in vivo tumor development assays, 5×10⁶ subconfluent ID8-Luc cells in 200 μl of 1×PBS were injected i.p. in C57BL/6 female mice.

Toxoplasma gondii Administration

Mice are injected intravaginally at day 15 with 5×10⁶ freshly isolated tachyzoites of RH-OVA strain of T. gondii.

Results

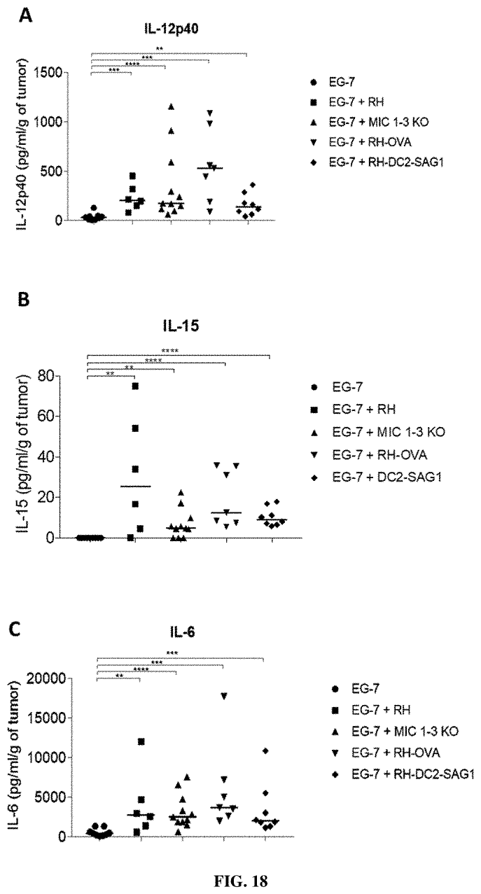

As shown in FIG. 17A, Toxoplasma treatment drastically inhibited growth and ascites formation associated with ovarian carcinoma in vivo compared to control.

Moreover, at postmortem examination, tumor weight was significantly decreased on the surface on omentum (FIG. 17B) suggesting that Toxoplasma gondii is potentially useful treatment for women with ovarian carcinoma.

### Example 7: Specific Targeting of Dendritic Cells by the Strain and In Vivo Effects

Materials and Methods

Parasites

T. gondii strain RH tachyzoites were produced in human fibroblasts (HFFs) cultured in Dulbecco's minimal medium (DMEM) supplemented with 10% of fetal calf serum, 2 mM glutamine, 50 U/ml of penicillin and 50 μ/ml of streptomycin. They were harvested during lysis of the host cells

Plasmid Construction of the RH-hPDL1Rec and NC-hPDL1Rec (recombinant anti-human PD1 ligand)

The anti-hPDL1Rec scFv results from the association of the heavy (IMGT 9814_H) and light (IMGT 9814_L) variable domains of Atezolizumab via a (Gly4Ser)3 peptide linker (SEQ ID NO: 55) and from a spacer GGGAS (SEQ ID NO: 28) in the C-terminal and a peptide HA tag in the N-terminal. The nucleotide and amino acid sequences of the optimized anti-hPDL1Rec scFv are given below.

Optimized anti-hPDL1Rec scFv nucleotide sequence
(SEQ ID NO: 56):
GAGGTGCAACTCGTCGAAAGCGGGGGCGGTTTGGTGCAACCTGGGGGAAGC
CTGCGGTTGTCTTGCGCCGCAAGCGGCTTTACGTTTTCCGATTCGTGGATT
CATTGGGTGAGACAAGCCCCAGGTAAGGGGCTCGAATGGGTGGCGTGGATC
AGTCCGTATGGTGGATCGACTTATTACGCGGACTCTGTGAAAGGAAGGTTT
ACAATCTCCGCGGATACGTCCAAAAATACCGCATATTTGCAGATGAATAGC
CTTCGCGCAGAGGACACAGCAGTTTATTACTGCGCCCGGAGACATTGGCCA
GGCGGCTTCGATTACTGGGGGCAAGGTACGCTGGTTACAGTTAGCAGCGGG
GGAGGAGGATCTGGGGGAGGTGGGTCGGGAGGGGGAGGTTCCGACATCCAA
ATGACTCAGTCGCCATCTAGTCTTTCTGCCTCTGTGGGGGATCGTGTTACC
ATCACGTGCCGTGCCAGCCAGGACGTTAGCACTGCTGTGGCCTGGTACCAG
CAAAAGCCGGGGAAGGCACCCAAACTTCTGATCTATAGTGCGTCGTTCCTC
TATAGTGGCGTTCCGTCGCGCTTCTCTGGTAGTGGCTCCGGCACCGACTTT
ACCCTGACAATCAGTAGCCTGCAGCCTGAGGACTTCGCTACCTATTATTGC
CAACAATACCTGTACCACCCGGCAACCTTCGGTCAAGGAACCAAAGTGGAG
ATTAAAGGAGGGGGGGCCAGTTCCAGA
Optimized VH anti-hPDL1Rec amino acid sequence (SEQ
ID NO: 57):
EVQLVESGGGLVQPGGSLRLSCAASGFTFSDSWIHWVRQAPGKGLEWVAWI
SPYGGSTYYADSVKGRFTISADTSKNTAYLQMNSLRAEDTAVYYCARRHWP
GGFDYWGQGTLVTVSSGGGGSGGGGSGGGGSDIQMTQSPSSLSASVGDRVT
ITCRASQDVSTAVAWYQQKPGKAPKLLIYSASFLYSGVPSRFSGSGSGTDF
TLTISSLQPEDFATYYCQQYLYHPATFGQGTKVEIKGGGASSR

As described hereabove for other recombinants, the anti-hPDL1Rec scFv is fused to SAG1. More precisely, the sequence encoding anti-hPDL1Rec SAG1 protein includes: the kozak sequence (consensus sequence for the initiation of the translation), the ATG, the sequence encoding the N-terminal signal sequence of SAG1, the HA tag, the VH region followed by the VL region of the anti-hPDL1Rec, the linker GGGAS, the sequence encoding a truncated SAG1 (without the N-terminal signal sequence) and the sequence encoding the SAG1 anchor signal (GPI=glycosylphosphatidylinositol) with a stop codon. This sequence is flanked in 5′ by a PmeI site and in 3′ by a NotI site. Thus, the pUC5 CAT/GFP-anti-hPDL1Rec/SAG1/GPI expresses the membrane-anchored fusion protein anti-hPDL1Rec-SAG1 under the control of promoter αTUB8 and the fusion protein CAT-GFP under the control of promoter αTUB5 to allow drug selection of stably transfected parasites (T. gondii strain RH or N. caninum tachyzoites).

ELISA on T. gondii Parasites

ELISA was performed on whole tachyzoites, essentially as described previously (Chardes et al., Infection and immunity, 1990). Flat bottomed wells (96-well plate, NUNC) were coated with 2×10⁵, 5×10⁵ or 10×10⁵ parasites/well in PBS. After centrifugation at 200×g and 4° C. for 5 min, 25 μL of 0.5% of glutaraldehyde in cold PBS was added to each well and left for 8 min at room temperature. The plates were washed twice in PBS and saturated with PBS-4% BSA for 1 h at 37° C. Rabbit anti-HA polyclonal antibodies (1:400, ThermoFisher Scientific) in PBS-1% BSA were incubated for 1 h at 37° C. After 3 washes in PBS-0.05% Tween, mouse monoclonal anti-rabbit IgG (γ-chain specific) alkaline phosphatase conjugate (1:4000 in PBS-BSA 1%, Sigma) was added to each well and incubated for 1 h at 37° C. After 3 washes with PBS-0.05% Tween, bound phosphatase activity was measured with p-nitrophenylphosphate (Sigma) (1 mg/ml in DEA-HCl 1 M bu.er pH 9.8).

Results

Cell Surface Expression of scFv-hPDL1Rec

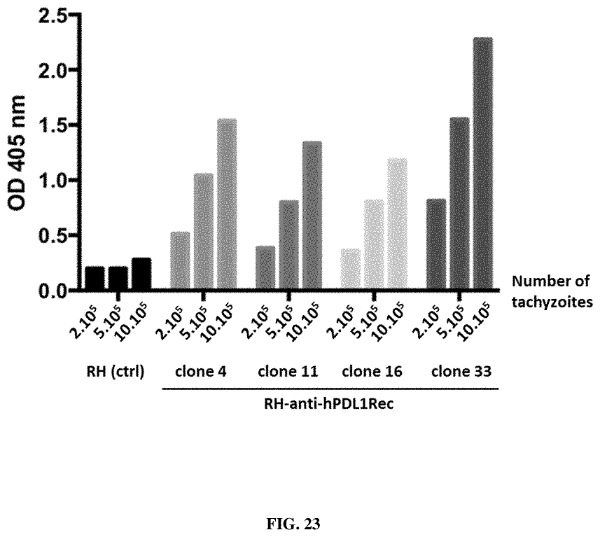

An ELISA analysis was performed on various selected clones of RH-anti-hPDL1Rec (FIG. 23). Briefly, T. gondii transfected with anti-hPDL1Rec were fixed with glutaraldehyde in flat bottomed wells. The anti-hPDL1Rec proteins were probed with rabbit anti-HA polyclonal antibodies. Results are expressed as optical density (OD).

As shown in FIG. 23, the scFv anti-hPDL1Rec was expressed in all T. gondii strains.

1-16. (canceled)

17. A strain of an Apicomplexa of the family Sarcocystidae, wherein said strain is replicative and expresses at least one heterologous gene or protein.

18. The strain according to claim 17, wherein the strain is Toxoplasma gondii or Neospora caninum.

19. The strain according to claim 17, wherein the strain expresses and/or secretes one or more heterologous protein(s) selected from the group comprising therapeutic molecules, antigens, recombinant surface receptors, or combinations thereof.

20. The strain according to claim 19, wherein the therapeutic molecule is a cytokine.

21. The strain according to claim 20, wherein the cytokine is a human IL15Rα sushi.

22. The strain according to claim 19, wherein the antigen is a cancer antigen or a neoantigen.

23. The strain according to claim 19, wherein the recombinant surface receptor comprises at least one extracellular-binding domain.

24. The strain according to claim 23, wherein the at least one extracellular-binding domain is an antigen-binding fragment or an antibody selected from the group comprising whole antibody, humanized antibody, single chain antibody, dimeric single chain antibody, Fv, scFv, Fab, F(ab)′2, defucosylated antibody, bi-specific antibody, diabody, triabody, tetrabody surface-exposed binding domain.

25. The strain according to claim 24, wherein the antigen-binding fragment or antibody is a scFV directed to DEC205.

26. The strain according to claim 17, wherein the strain is at a tachyzoite stage.

27. A composition or a pharmaceutical composition comprising the strain according to claim 17, wherein said pharmaceutical composition further comprises at least one pharmaceutically acceptable excipient.

28. The composition according to claim 27, in combination with a therapeutic protein or molecule.

29. The composition according to claim 27, wherein said composition is a vaccine composition.

30. The composition according to claim 29, wherein said vaccine composition comprises an adjuvant.

31. A method of preventing and/or treating cancer or a chronic infectious disease in a subject in need thereof, wherein said chronic infectious disease is selected from chronic virus infection and chronic bacterial infection, said method comprising administering to said subject a therapeutically effective amount of the strain according to claim 17, or a composition, pharmaceutical composition or vaccine composition comprising said strain.

32. The method according to claim 31, wherein said cancer is a solid tumor.

33. The method according to claim 31, wherein said cancer is an ovarian cancer, pancreatic cancer, lung cancer, melanoma or glioblastoma.

34. The method according to claim 31, wherein said chronic infectious disease is associated with or induces an immunosuppression, and is selected from the group consisting of tuberculosis and HIV.

35. A method of producing at least one heterologous protein by a strain according to claim 17, said method comprising:
a) infecting a cell with the strain, wherein the strain secretes said at least one heterologous protein,
b) cultivating the infected cell of a) in a culture medium, and
c) recovering the least one heterologous protein.
